(19) United States
(12) Patent Application Publication
(10) Pub. No.: US 2003/0170633 A1
(43) Pub. Date: Sep. 11, 2003

(54) Adenoviral library assay for adipogenesis genes and methods and compositions for screening compounds

(76) Inventors: Ronald Vogels, Linschoten (NL); Abraham Bout, Moerkapelle (NL); Helmuth van Es, Hoofddorp (NL); Govert Schouten, Leidenderp (NL); Luc Van Rompaey, Keerbergen (BE)

Correspondence Address: SYNNESTVEDT & LECHNER, LLP, 2600 ARAMARK TOWER, 1101 MARKET STREET, PHILADELPHIA, PA, 191072950

(21) Appl. No.: 10/074,668

(22) Filed: Feb. 13, 2002

Related U.S. Application Data
(63) Continuation-in-part of application No. 10/036,949, filed on Dec. 21, 2001.
Application No. 10/036,949 is a division of application No. 09/358,036, filed on Jul. 21, 1999, now Pat. No. 6,340,595.
Application No. 09/358,036 is a continuation-in-part of application No. 09/097,239, filed on Jun. 12, 1998, now Pat. No. 6,413,776.

(51) Int. Cl. C12Q 1/68; C12P 7/64; C12N 15/861

(52) U.S. Cl. 435/6; 435/456; 435/134

(57) ABSTRACT

Methods, and compositions for use therein, for directly, rapidly, and unambiguously identifying, in a high throughput setting, unique nucleic acids involved in the process of lipid vacuole formation in cells and/or the cell differentiation process of adipogenesis, using an adenoviral vector library system. The method identifies unique nucleic acids capable of inducing lipid droplet formation in a cell, and determines whether the expression product of such a nucleic acid is secreted. Drug candidate compounds useful in the treatment of disease states such as obesity, type II diabetes and hyperglycemia are identified by the screening of compounds that either increase or decrease the formation of lipid droplets, or mRNA expression in host cells. Pharmaceutical compositions and methods of treatment comprising the polypeptides or polynucleotides identified by the methods of the present invention are disclosed.

### CROSS-REFERENCE TO RELATED APPLICATION

This application is a continuation-in-part of U.S. patent application Ser. No. 10/036,949, filed on Dec. 21, 2001, which is a divisional of U.S. patent application Ser. No. 09/358,036, filed on Jul. 21, 1999, now U.S. Pat. No. 6,340,595, which is a continuation-in-part of pending U.S. patent application Ser. No. 09/097,239, filed on Jun. 12, 1998.

### FIELD OF THE INVENTION

The invention relates to high throughput methods for identifying the function of sample nucleic acids and their products.

The ultimate goal of the Human Genome Project is to sequence the entire human genome. The expected outcome of this effort is a precise map of the 70,000-100,000 genes that are expressed in man. Since the early 1980s, a large number of Expressed Sequence Tags (ESTs), which are partial DNA sequences read from the ends of complementary DNA (cDNA) molecules, have been obtained by both government and private research organizations. A hallmark of these endeavors, carried out by a collaboration between Washington University Genome Sequencing Center and members of the IMAGE (Integrated Molecular Analysis of Gene Expression) consortium (http:/www-bio.llnl.gov/bbrp/image/image.html), has been the rapid deposition of the sequences into the public domain and the concomitant availability of the sequence-tagged cDNA clones from several distributors (Marra, et al. (1998) Trends Genet. 14(1):4-7). At present, the collection of cDNAs is believed to represent approximately 50,000 different human genes expressed in a variety of tissues including liver, brain, spleen, B-cells, kidney, muscle, heart, alimentary tract, retina, and hypothalamus, and the number is growing daily.

Recent initiatives like that of the Cancer Genome Anatomy project support an effort to obtain full-length sequences of clones in the Unigene set (a set of cDNA clones that is publicly available). At the same time, commercial entities propose to validate 40,000 full-length cDNA clones. These individual clones will then be available to any interested party. The speed by which the coding sequences of novel genes are identified is in sharp contrast to the rate by which the function of these genes is elucidated. Assigning functions to the cDNAs in the databases, or functional genomics, is a major challenge in biotechnology today.

For decades, novel genes were identified as a result of research designed to explain a biological process or hereditary disease and the function of the gene preceded its identification. In functional genomics, coding sequences of genes are first cloned and sequenced and the sequences are then used to find functions. Although other organisms such as Drosophila, C. elegans, and Zebrafish are highly useful for the analysis of fundamental genes, animal model systems are inevitable for complex mammalian physiological traits (blood glucose, cardiovascular disease, inflammation). However, the slow rate of reproduction and the high housing costs of the animal models are a major limitation to high throughput functional analysis of genes. Although labor intensive efforts are made to establish libraries of mouse strains with chemically or genetically mutated genes in a search for phenotypes that allow the elucidation of gene function or that are related to human diseases, a systematic analysis of the complete spectrum of mammalian genes, be it human or animal, is a significant task.

In order to keep pace with the volume of sequence data, the field of functional genomics needs the ability to perform high throughput analysis of true gene function. Recently, a number of techniques have been developed that are designed to link tissue and cell specific gene expression to gene function. These include cDNA microarraying and gene chip technology and differential display messenger RNA (mRNA). Serial Analysis of Gene Expression (SAGE) or differential display of mRNA can identify genes that are expressed in tumor tissue but are absent in the respective normal or healthy tissue. In this way, potential genes with regulatory functions can be separated from the excess of ubiquitously expressed genes that have a less likely chance to be useful for small drug screening or gene therapy projects. Gene chip technology has the potential to allow the monitoring of gene expression through the measurement of mRNA expression levels in cells of a large number of genes in only a few hours. Cells cultured under a variety of conditions can be analyzed for their mRNA expression patterns and compared to provide insight into their function and relationship to disease states.

Recent functional genomics investigations into the underlying genetic basis of obesity and related disease states are revealing a complex interplay of protein-protein interactions. Obesity is a multi-factorial syndrome representing one of the most important pathological states in western countries. This condition is associated with hypertension, diabetes, cardiovascular problems, and certain types of cancers. Obesity is characterized by an increase in body fat stores linked to a lack of control on food intake and/or energy expenditure (Kopelman, (2000) Nature 404(6778):635-43). There are at least four mechanisms reported in the literature by which drugs can fight obesity: 1) reducing the amount of fat absorbed, 2) increasing fat metabolism, 3) curbing appetite, and 4) resetting the central controls of body weight. The exploitation of various metabolic pathways has been proposed to treat obesity (Dove, (2001) Nat. Biotechnol. 19(1):25-8; Spiegelman and Flier, (2001) Cell 104(4):531-43). Different classes of anti-obesity drugs and their mechanism of action are discussed in the review by Campfield, et al. (1998) Science 280(5368): 1383-7.

Obesity and the leptin protein appear to have a role in bone homeostasis (Ducy, et al. (2000) Cell 100(2): 197-207). It has been reported that PPARγ regulates (inhibits) osteoclast differentiation, and inhibits osteoclast bone resorbing activity (Mbalaviele, et al. (2000) J. Biol. Chem. 275(19):14388-93). PPARγ has also been linked to TNFα, which has been implicated in the regulation of adipogenesis, obesity and insulin resistance. In Bullo-Bonet, et al. (1999) FEBS Lett 451(3):215-9, the reviewers summarize the effects TNFα has on adipogenesis, including the inhibition of enzymes involved in fat synthesis in adipose tissue, and the promotion of insulin resistance by inactivating insulin signaling by a mechanism that includes serine-phosphorylation of IRS1 (insulin receptor substrate-1). The reviewers also reported that TNFα signaling may also inhibit the kinase activity of the insulin receptor, thus further abrogating the effects of insulin on the cell. TNFα appears to down regulate PPARγ and C/EBP family members, thus providing a putative mechanism by which it exerts its effects on adipocytes (Moller, (2000) Trends Endocrinol. Metab. 1 1(6):212-7). It has also been reported that C/EBP family members induce the murine gene sequence FSP27, which is expressed during adipocyte differentiation (Danesch, et al. (1992) J. Biol. Chem. 267(10):7185-93).

It has been reported that PPARγ is expressed in liposarcomas and that the maximal activation of PPARγ may in some cases overcome the neoplastic phenotype (Tontonoz, et al. (1997) Proc. Natl. Acad. Sci. USA 94(1):237-41). Furthermore, activation of PPARγ by TZDs has been reported to differentiate breast adenocarcinomas to a less malignant state (Mueller, et al. (1998) Mol. Cell 1(3):465-70). It has further been reported that PPARy activation reverses a proliferative phenotype of adipocytes cultured in dilipidated medium, and can overcome retinoic acid-induced apoptosis of these cells. Thus, in addition to its pro-diferentiative and anti-proliferative activities, PPARγ appears to promote survival (Chawla, et al. (1994) Proc. Natl. Acad. Sci. USA 91(5):1786-90).

PPARγ activators and the effects (increased insulin sensitivity, differentiation of adipocytes) are among the many methods and strategies being investigated to fight Type II diabetes (Saltiel, (2001) Cell 104(4):517-29). Ligands for PPARγ have been reported to increase insulin sensitivity without increasing obesity (Rocchi, et al. (2001) Mol. Cell 8(4):737-47). One such ligand, 15-deoxy-delta 12, 14-prostaglandin J2 is reported to induce adipogenesis (Forman, et al. (1995) Cell 83(5):803-12). The inflammatory cytokine leukotriene B4 (LTB4) is a natural agonist both PPARγ and the seven transmembrane receptor BLTR. Compounds that bind to both proteins, and differentially modulate their activity have been reported (Devchand, et al. (1999) J. Biol. Chem. 274(33):23341-8). Thiazolidinediones (TZDs) are a class of drugs that activate PPARγ (peroxisome proliferator-activated receptor γ). It has been reported that PPARγ activation results in accumulation of adipose tissue and an increased sensitization of adipose tissue to insulin. However, investigators speculate that this increased sensitization may only be transient and once a critical adipose tissue threshold has been reached insulin resistance may return (Schoonjans, et al. (2000) Lancet 355(9208): 1008-10).

Reported Developments

DNA microarray chips with 40,000 non-redundant human genes have been produced and were projected to be on the market in 1999 (Editorial, (1998) Nat. Genet. 18(3): 195-7). However, these techniques are primarily designed for screening cancer cells and not for screening for specific gene functions.

Double or triple hybrid systems also are used to add functional data to the genomic databases. These techniques assay for protein-protein, protein-RNA, or protein-DNA interactions in yeast or mammalian cells (Brent and Finley, (1997) Annu. Rev. Genet. 31:663-704). However, this technology does not provide a means to assay for a large number of other gene functions such as differentiation, motility, signal transduction, and enzyme and transport activity.

Yeast expression systems have been developed which are used to screen for naturally secreted and membrane proteins of mammalian origin (Klein, et al. (1996) Proc. Natl. Acad. Sci. USA 93(14):7108-13). This system also allows for collapsing of large libraries into libraries with certain characteristics that aid in the identification of specific genes and gene products. One disadvantage of this system is that genes encoding secreted proteins are primarily selected. A second disadvantage is that the library may be biased because the technology is based on yeast as a heterologous expression system and there will be gene products that are not appropriately folded.

The development of high throughput screens is discussed in Jayawickreme and Kost, (1997) Curr. Opin. Biotechnol. 8:629-634. A high throughput screen for rarely transcribed differentially expressed genes is described in von Stein, et al. (1997) Nucleic Acids Res. 35:2598-2602. High throughput genotyping is disclosed in Hall, et al. (1996) Genome Res. 6:781-790. Methods for screening transdominant intracellular effector peptides and RNA molecules are disclosed in Nolan, WO 97/27212 and WO 97/27213.

Other current strategies include the creation of transgenic mice or knockout mice. A successful example of gene discovery by such an approach is the identification of the osteoprotegerin gene. DNA databases were screened to select ESTs with features suggesting that the cognate genes encoded secreted proteins. The biological functions of the genes were assessed by placing the corresponding full-length cDNAs under the control of a liver-specific promoter. Transgenic mice created with each of these constructs consequently have high plasma levels of the relevant protein. Subsequently, the transgenic animals were subjected to a battery of qualitative and quantitative phenotypic investigations. One of the genes that was transfected into mice produced mice with an increased bone density, which led subsequently to the discovery of a potent anti-osteoporosis factor (Simonet, et al. (1997) Cell 89(2):309-19). The disadvantages of this method are that the method is costly and highly time consuming.

The challenge in functional genomics is to develop and refine all the above-described techniques and integrate their results with existing data in a well-developed database that provides for the development of a picture of how gene function constitutes cellular metabolism and a means for this knowledge to be put to use in the development of novel medicinal products. The current technologies have limitations and do not necessarily result in true functional data. Therefore, there is a need for a method that allows for direct measurement of the function of a single gene from a collection of genes (gene pools or individual clones) in a high throughput setting in appropriate in vitro assay systems and animal models. A method for identifying genes having adipogenesis or obesity-related function(s) from a large array of gene sequences has not been reported.

### SUMMARY OF THE INVENTION

The present invention relates to methods, and compositions for use therein, for directly, rapidly, and unambiguously identifying, in a high throughput setting, unique nucleic acids involved in the process of lipid vacuole formation in cells and/or the cell differentiation process of adipogenesis, using an adenoviral vector library system. More particularly, the present invention relates to a method of identifying a unique nucleic acid capable of inducing lipid droplet formation in a cell, wherein said unique nucleic acid is present in a library, said method comprising: (a) providing a library of a multitude of unique expressible nucleic acids, said library including a multiplicity of compartments, each of said compartments consisting essentially of one or more adenoviral vector comprising at least one unique nucleic acid of said library in an aqueous medium, wherein said adenoviral vector is capable of introducing said nucleic acid into a host cell, is capable of expressing the product of said nucleic acid in said host cell, and is deleted in a portion of the adenoviral genome necessary for replication thereof in said host cell; (b) transducing a multiplicity of host cells with at least one adenoviral vector comprising at least one unique nucleic acid from said library; (c) incubating said host cells to allow expression of the product of said nucleic acid; and (d) determining if a lipid droplet is formed in said cell. The host cell transduced with said recombinant adenoviral vector is observed for the formation of lipid droplets, and if such droplets are formed, an adipogenesis-related function is assigned to the product(s) encoded by the sample nucleic acids.

The present method also comprises: (a) growing a plurality of cell cultures containing at least one cell, said one cell expressing adenoviral sequence consisting essentially of E1-region sequences and expressing one or more functional gene products encoded by at least one adenoviral region selected from an E2A region and an E4 region; (b) transfecting, under conditions whereby said recombinant adenovirus vector library is produced, said at least one cell in each of said plurality of cell cultures with

i) an adapter plasmid comprising adenoviral sequence coding, in operable configuration, for a functional Inverted Terminal Repeat, a functional encapsidation signal, and sequences sufficient to allow for homologous recombination with a first recombinant nucleic acid, and not coding for E1 region sequences which overlap with E1 region sequences in said at least one cell, for E1 region sequences which overlap with E1 region sequences in a first recombinant nucleic acid, for E2B region sequences other than essential E2B sequences, for E2A region sequences, for E3 region sequences and for E4 region sequences, and further comprises a unique nucleic acid sequence and promoter operatively linked to said unique nucleic acid sequence; and

ii) a first recombinant nucleic acid comprising adenoviral sequence coding, in operable configuration, for a functional adenoviral Inverted Terminal Repeat and for sequences sufficient for replication in said at least one cell, but not comprising adenoviral E1 region sequences which overlap with E1 sequences in said at least one cell, and not comprising E2A region sequences or E4 region sequences expressed in said plurality of cells which would otherwise lead to production of replication competent adenovirus wherein said first recombinant nucleic acid has sufficient overlap with said adapter plasmid to provide for homologous recombination resulting in production of recombinant adenoviral vectors in said at least one cell;

(c) incubating said plurality of cells under conditions which result in the lysis of said plurality of cells facilitating the release of said recombinant adenoviral vectors containing said unique nucleic acid; (d) transferring an aliquot of said adenoviral vectors into a corresponding plurality of host cell cultures consisting of cells in which said vectors do not replicate, but in which said nucleic acids are expressible; (e) incubating said host cells to allow expression of the product of said nucleic acid; and (f) observing said host cell for the presence of a lipid droplet.

A further aspect of the present assay methods is determining whether the expression product of the nucleic acid capable of inducing lipid droplet formation is secreted by said cell, comprising: (a) infecting producer cells in a medium with an adenoviral vector comprising a unique nucleic acid capable of inducing lipid droplet formation; (b) combining said medium with test cells that have not been infected with said vector; and (c) determining if lipid droplets are formed in said test cells.

Another aspect of the present invention relates to a method for identifying a drug candidate compound useful in the treatment of a disease state, said method comprising: (a) contacting a first subpopulation of host cells transfected with polynucleotide, identified in the above-described method of the invention, with one or more of said test compound, and (b) identifying, from said one or more test compounds, a candidate compound that inhibits or enhances the formation of lipid droplets in said first subpopulation of transfected host cells relative to a second subpopulation of said transfected host cells that have not been contacted with said test compound.

Another means of detecting candidate compounds comprises selecting a compound that induces either an increase or decrease in the expression of mRNA encoded by a polynucleotide comprising a sequence of SEQ ID NO: 14 or SEQ ID NO: 16 in said first subpopulation of transfected host cell relative to the expression of said mRNA in a second subpopulation of transfected host cells that has not been contacted with such compound.

A further aspect of the present method comprises first determining the binding affinity of said one or more test compound to (1) the polynucleotide identified in accordance with the present method invention, or (2) the corresponding antisense sequences thereof, or (3) an expression product said sequences, by contacting one or more test compound therewith.

The present method is useful for identifying compounds that are suitable as drug candidate compounds, the pharmaceutical application of which is related to whether the aforesaid assay results in either an increase or a decrease in the formation of lipid droplets, or the mRNA expression of the above-identified polynucleotides, in the host cells. If a test compound inhibits lipid droplet formation, then the compound is useful for the treatment of obesity. On the other hand, if a test compound enhances lipid droplet formation, then the compound may be useful for the treatment of a disease state selected from the group consisting of type II diabetes, hyperglycemia, impaired glucose tolerance, metabolic syndrome, syndrome X, dyslipidemia and insulin resistance.

The present invention also relates to pharmaceutical compositions and methods of treatment comprising the polypeptides or polynucleotides described hereinbelow. Other aspects and more detailed description of the present invention are provided in the following sections.

### BRIEF DESCRIPTION OF THE DRAWINGS

FIG. 1: Construction of pBS.PGK.PCRI. pBS.PGK.PCRI encodes the human phosphoglycerate kinase (PGK) promoter operatively linked to adenovirus 5 (Ad5) E1 nucleotides 459-916. To construct this plasmid, Ad5 nucleotides 459-916 are amplified by the polymerase chain reaction (PCR) with primers Ea-1 (SEQ ID NO: 1) and Ea-2 (SEQ ID NO: 2), digested with Cla I, and cloned into the ClaI-EcoRV sites of pBluescript (Stratagene), resulting in pBS.PCRI. The PGK promoter is excised from pTN by complete digestion with ScaI and partial digestion with EcoRI and cloned into the corresponding sites of pBS.PCRI, resulting in pBS.PGK.PCRI.

FIG. 2: Construction of pIG.E1A.E1B.X. pIG.E1A.E1B.X encodes Ad5 nucleotides 459-5788 (E1A and E1B regions) operatively linked to the human PGK promoter. pIG.E1A.E1B.X also encodes Ad5 pIX protein. pIG.E1A.E1B.X is constructed by replacing the ScaI-BspEI fragment of pAT-X/S with the corresponding fragment of pBS.PGK.PCRI.

FIG. 3A: Construction of pAT-PCR2-NEO. To construct this plasmid, the E1B promoter and initiation codon (ATG) of the E1B 21 kDa protein are PCR amplified with primers Ea-3 (SEQ ID NO: 3) and Ep-2 (SEQ ID NO: 4), where Ep-2 introduces an NcoI site (5′-CCATGG) at the 21 kDa protein initiation codon. The PCR product (PCRII) is digested with HpaI and NcoI and ligated into the corresponding sites of pAT-X/S, producing pAT-X/S-PCR2. The NcoI-StuI fragment of pTN, containing the Neo^{R} and a portion of the HBV poly(A) site are ligated into the NcoI-NruI sites of pAT-X/S-PCR2, producing pAT-PCR2-NEO.

FIG. 3B: Construction of pIG.E1A.NEO. pIG.E1A.NEO encodes Ad5 nucleotides 459-1713 operatively linked to the human PGK promoter. Also encoded is the E1B promoter functionally linked to the neomycin resistance gene (Neo^{R}) and the hepatitis B virus (HBV) poly(A) signal. In this construct, the AUG codon of the E1B 21 kDa protein functions as the initiation codon of Neo^{R}. The HBV poly(A) signal of pAT-PCR2-NEO (see FIG. 3A) is completed by replacing the ScaI-SalI fragment of pAT-PCR2-NEO with the corresponding fragment of pTN, producing pAT.PCR2.NEO.p(A), and replacing the ScaI-XbaI fragment of pAT.PCR2.NEO.p(A) with the corresponding fragment of pIG.E1A.E1B.X, producing pIG.E1A.NEO.

FIG. 4: Construction of pIG.E1A.E1B. pIG.E1A.E1B contains the Ad5 nucleotides 459-3510 (E1A and E1B proteins) operatively linked to the PGK promoter and HBV poly(A) signal. This plasmid is constructed by PCR amplification of the N-terminal amino acids of the E1B 55 kDa protein with primers Eb-1 (SEQ ID NO: 5) and Eb-2 (SEQ ID NO: 6), which introduces an XhoI site, digested with Bg/II and cloned into the Bg/II-NruI sites of pAT-X/S, producing pAT-PCR3. The XbaI-XhoI fragment of pAT-PCR3 is replaced with the XbaI-SalI fragment (containing the HBV poly(A) site) of pIG.E1A.NEO to produce pIG.E1A.E1B.

FIG. 5: Construction of pIG.NEO. pIG.NEO contains the Neo^{R} operatively linked to the E1B promoter. pIG.NEO was constructed by ligating the HpaI-ScaI fragment of pAT.PCR2.NEO.p(A) or pIG.E1A.NEO, which contains the E1B promoter and Neo^{R} into the EcoRV-ScaI sites of pBS.

FIG. 6: Transformation of primary baby rat kidney (BRK) cells by adenoviral packaging constructs. Subconfluent dishes of BRK cells are transfected with 1 or 5 μg of either pIG.NEO, pIG.E1A.NEO, pIG.E1A.E1B, pIG.E1A.E1B.X, pAd5XhoIC, or pIG.E1A.NEO plus pDC26, which expresses the Ad5 E1B gene under control of the SV40 early promoter. Three weeks post-transfection, foci are visible, cells are fixed, Giemsa stained and the foci counted. The results shown are the average number of foci per 5 replicate dishes.

FIG. 7: Western blot analysis of A549 clones transfected with pIG.E1A.NEO and human embryonic retinoblasts (HER) cells transfected with pIG.E1A.E1B (PER clones). Expression of Ad5 E1A and E1B 55 kDa and 21 kDa proteins in transfected A549 cells and PER cells is determined by Western blot with mouse monoclonal antibodies (Mab) M73, which recognizes E1A gene products, and Mabs AIC6 and C1G11, which recognize the E1B 55 kDa and 21 kDa proteins, respectively. Mab binding is visualized using horseradish peroxidase-labelled goat anti-mouse antibody and enhanced chemiluminesence. 293 and 911 cells served as controls.

FIG. 8: Southern blot analysis of 293, 911 and PER cell lines. Cellular DNA is extracted, HindIII digested, electrophoresed, and transferred to Hybond N+ membranes (Amersham). Membranes are hybridized to radiolabelled probes generated by random priming of the SspI-HindIII fragment of pAd5.SalB (Ad5 nucleotides 342-2805).

FIG. 9: Transfection efficiency of PER.C3, PER.C5, PER.C6 and 911 cells. Cells are cultured in 6-well plates and transfected in duplicate with 5 μg pRSV.lacZ by calcium-phosphate co-precipitation. Forty-eight hours post-transfection, cells are stained with X-Gal, and blue cells are counted. Results shown are the mean percentage of blue cells per well.

FIG. 10: Construction of adenoviral vector, pMLPI.TK. pMLPI.TK is designed to have no sequence overlap with the packaging construct pIG.E1A.E1B. pMLPI.TK is derived from pMLP.TK by deletion of the region of sequence overlap with pIG.E1A.E1B and deletion of non-coding sequences derived from lacZ. SV40 poly(A) sequences of pMLP.TK are PCR amplified with primers SV40-1 (SEQ ID NO: 7), which introduces a BamHI site, and SV40-2 (SEQ ID NO: 8), which introduces a BglII site. pMLP.TK Ad5 sequences 2496 to 2779 are PCR amplified with primers Ad5-1 (SEQ ID NO: 9), which introduces a Bg/II site, and Ad5-2 (SEQ ID NO: 10). Both PCR products are Bg/II digested, ligated, and PCR amplified with primers SV40-1 and Ad5-2. This third PCR product is BamHI and AflIII digested and ligated into the corresponding sites of pMLP.TK, producing pMLPI.TK.

FIG. 11A: New adenoviral packaging construct, pIG.E1A.E1B, does not have sequence overlap with new adenoviral vector, pMLPI.TK. Regions of sequence overlap between the packaging construct pAd5XhoIC, expressed in 911 cells, and adenoviral vector pMLP.TK, that can result in homologous recombination and the formation of RCA are shown. In contrast, there are no regions of sequence overlap between the new packaging construct pIG.E1A.E1B, expressed in PER.C6 cells, and the new adenoviral vector pMLPI.TK.

FIG. 11B: New adenoviral packaging construct pIG.E1A.NEO, does not have sequence overlap with new adenoviral vector pMLPI.TK. There are no regions of sequence overlap between the new packaging construct pIG.E1A.NEO and the new adenoviral vector pMLPI.TK that can result in homologous recombination and the formation of RCA.

FIG. 12: Generation of recombinant adenovirus, IG.Ad.MLPI.TK. Recombinant adenovirus IG.Ad.MLPI.TK is generated by co-transfection of 293 cells with SalI linearized pMLPI.TK and the right arm of ClaI digested, wild-type Ad5 DNA. Homologous recombination between linearized pMLPI.TK and wild-type Ad5 DNA produces IG.Ad.MLPI.TK DNA, which contains an E1 deletion of nucleotides 459-3510. 293 cells transcomplement the deleted Ad5 genome, thereby permitting replication of the IG.Ad.MLPI.TK DNA and its packaging into virus particles.

FIG. 13: Rationale for the design of adenoviral-derived recombinant DNA molecules that duplicate and replicate in cells expressing adenoviral replication proteins. A diagram of the adenoviral double-stranded DNA genome indicating the approximate locations of E1, E2, E3, E4, and L regions is shown. The terminal polypeptide (TP) attached to the 5′-terminus is indicated by closed circles. The right arm of the adenoviral genome can be purified by removal of the left arm by restriction enzyme digestion. Following transfection of the right arm into 293 or 911 cells, adenoviral DNA polymerase (white arrow) encoded on the right arm will produce only single-stranded forms. Neither the double-stranded or single-stranded DNA can replicate because they lack an inverted terminal repeat (ITR) at one terminus. Providing the single-stranded DNA with a sequence that can form a hairpin structure at the 3′-terminus, which serves as a substrate for DNA polymerase, will extend the hairpin structure along the entire length of the molecule. This molecule can also serve as a substrate for a DNA polymerase, but the product is a duplicated molecule with ITRs at both termini that can replicate in the presence of adenoviral proteins.

FIG. 14: Adenoviral genome replication. The adenoviral genome is shown in the top left panel. The origins or replication are located within the left and right ITRs at the genome ends. DNA replication occurs in two stages. Replication proceeds from one ITR, generating a daughter duplex and a displaced parental single-strand that is coated with adenoviral DNA binding protein (DBP, open circles) and can form a panhandle structure by annealing of the ITR sequences at both termini. The panhandle is a substrate for DNA polymerase (Pol: white arrows) to produce double-stranded genomic DNA. Alternatively, replication proceeds from both ITRs, generating two daughter molecules, thereby obviating the requirement for a panhandle structure.

FIG. 15: Potential hairpin conformation of a single-stranded DNA molecule that contains the HP/asp sequence (SEQ ID NO: 11). Asp718I digestion of pICLha, containing the cloned oligonucleotides HP/asp1 and HP/asp2, yields a linear double-stranded DNA with an Ad5 ITR at one terminus and the HP/asp sequence at the other terminus. In cells expressing the adenoviral E2 region, a single-stranded DNA is produced with an Ad5 ITR at the 5′-terminus and the hairpin conformation at the 3′-terminus. Once formed, the hairpin can serve as a primer for cellular and/or adenoviral DNA polymerase to convert the single stranded DNA to double stranded DNA.

FIG. 16: Diagram of pICLhac. pICLhac contains all the elements of pICL (FIG. 19) but also contains the HP/asp sequence in the Asp718 site in an orientation that will produce the hairpin structure shown in FIG. 15, following linearization by Asp718 digestion and transfection into cells expressing adenoviral E2 proteins.

FIG. 17: Diagram of pICLhaw. pICLhaw is identical to pICLhac (FIG. 16) except that the inserted HP/asp sequence is in the opposite orientation.

FIG. 18: Schematic representation of pICLI. pICLI contains all the elements of pICL (FIG. 19) but also contains an Ad5 ITR in the Asp718 site.

FIG. 19: Diagram of pICL. pICL is derived from the following: (i) nucleotides 1-457, Ad5 nucleotides 1-457 including the left ITR, (ii) nucleotides 458-969, human Cytomegalovirus (CMV) enhancer and immediate early promoter, (iii) nucleotides 970-1204, SV40 19S exon and truncated 16/19S intron, (iv) nucleotides 1218-2987, firefly luciferase gene, (v) nucleotides 3018-3131, SV40 tandem polyadenylation signals from the late transcript, (vi) nucleotides 3132-5620, pUC12 sequences including an Asp718 site, and (vii) ampicillin resistance gene in reverse orientation.

FIG. 20: Shows a schematic overview of the adenoviral fragments cloned in pBr322 (plasmid) or pWE15 (cosmid) derived vectors. The top line depicts the complete adenoviral genome flanked by its ITRs (filled rectangles) and with some restriction sites indicated. Numbers following restriction sites indicate approximate digestion sites (in kb) in the Ad5 genome.

FIG. 21: Drawing of adapter plasmid pAd/L420-HSA

FIG. 22: Drawing of adapter plasmid pAd/Clip

FIG. 23: Schematic representation of the generation of recombinant adenoviruses using a plasmid-based system. In the top of the figure, the genome organization of Ad5 is shown with filled boxes representing the different early and late transcription regions and flanking ITRs. The middle of the figure represents the two DNAs used for a single homologous recombination while the bottom of the figure represents the recombinant virus after transfection into packaging cells.

FIG. 24: Drawing of minimal adenoviral vector pMV/L420H

FIG. 25: Helper construct for replication and packaging of minimal adenoviral vectors. Schematic representation of the cloning steps for the generation of the helper construct pWE/AdΔ5′.

FIG. 26: Evidence for SV40-LargeT/ori mediated replication of large adenoviral constructs in COS-1 cells. FIG. 26A shows a schematic representation of construct pWE/Ad.Δ5′. The location of the SV40 ori sequence and the fragments used to prepare probes are indicated. Evidence for SV40-LargeT/ori mediated replication of large adenoviral constructs in COS-1 cells. FIG. 26B shows an autoradiogram of the Southern blot hybridized to the adenoviral probe. FIG. 26C shows an autoradiogram of the Southern blot hybridized to the pWE probe. Lane 1, marker lane: λ DNA digested with EcoRI and HindIII. Lane 4 is empty. Lanes 2, 5, 7, 9, 11, 13, 15, and 17 contain undigested DNA and Lanes 3, 6, 8, 10, 12, 14, 16 and 18 contain MboI digested DNA. All lanes contain DNA from COS-1 cells transfected with pWE.pac (lanes 2 and 3), pWE/Ad.Δ5 ′ construct #1 (lanes 5 and 6), #5 (lanes 7 and 8) and #9 (lanes 9 and 10), pWE/Ad.AflII-rITR (lanes 11 and 12), pMV/CMV-LacZ (lanes 13 and 14), pWE.pac digested with PacI (lanes 15 and 16), or pWE/Ad.AflII-rITR digested with PacI (lanes 17 and 18) as described in the text. Arrows point to the expected positive signal of 1416 bp (FIG. 26B) and 887 bp (FIG. 26C).

FIG. 27: Production of E1/E2A deleted adenoviral vectors and its efficiency in miniaturized PER. C6/E2A based production system.

FIG. 28: Average titers produced in a 96-well plate as measured using a PER. C6/E2A based plaque assay.

FIG. 29: Fidelity of adenoviral vector production miniaturized PER.C6/E2A based production system for a number of marker and human cDNA transgenes.

FIG. 30: Percentage of wells showing CPE formation after transfection of PER.C6/E2A cells with pCLIP-LacZ, purified by 6 different protocols. Qiagen: standard alkaline lysis followed by Qiagen plasmid purification; AlkLys: alkaline lysis followed by isopropanol precipitation, and solubilization in TE buffer; AL+RNase: alkaline lysis followed by isopropanol precipitation, and solubilization in TE buffer containing RNase at 10 microgram per ml; AL+R+phenol: alkaline lysis followed by isopropanol precipitation, and solubilization in TE buffer containing RNase at 10 microgram per ml, followed by phenol/chloroform extraction and ethanol precipitation; cetyltrimethylammoniumbromide (CTAB): Standard CTAB plasmid isolation; CTAB+phenol: Standard CTAB plasmid isolation, but solubilization in TE buffer containing RNase at 10 microgram per ml, followed by phenol/chloroform extraction.

FIG. 31: Effect of using digested plasmid for transfection without phenol-chloroform extraction. The results of all experiments are depicted and expressed as percentage of wells showing CPE formation. A) LacZ-adapter DNA is isolated using 6 different isolation methods; 1: Qiagen, 2: Alkaline lysis, 3: Alkaline lysis+RNase treatment, 4: Alkaline lysis+RNase treatment+p/c purification of DNA before linearization, 5: cetyltrimethylammoniumbromide (CTAB), 6: CTAB+p/c purification of DNA before linearization, rITR is p/c purified, B) Purified and unpurified EGFP- and EYFP-adapter DNA, rITR is p/c purified, C) EGFP-adapter DNA and rITR are tested purified and unpurified; 1: Both adapter and rITR purified (control), 2: rITR purified, adapter DNA unpurified, 3: rITR and adapter unpurified.

FIG. 32: Stability of adenoviral vectors produced in miniaturized format and incubated for up to three weeks at three different temperatures and measured using a plaque forming assay for adenoviral vectors.

FIG. 33: Efficiencies of virus generation in percentages of CPE after virus generation of several adenoviruses (E1 and E2A deleted) containing cDNAs in antisense (AS) orientation.

FIG. 34A-M: Plasmid maps of adenoviral adapter plasmids. These adenoviral adapter plasmids are particularly useful for the construction of expression libraries in adenoviral vectors such as the subject of this application. They have very rare restriction sites for the linearization of adapters and libraries of adapters (with transgenes inserted) and will not inactivate the adapter by digestion of the inserts. In FIG. 34M, the cosmid containing pIPspAdapt5- or pCLIP-IppoI-polynew-derived adenoviral DNA can be used for in vitro ligations. Double stranded oligonucleotides containing compatible overhangs are ligated between the I-CeuI and PI-SceI sites, between I-CeuI and I-PpoI, between I-SceI and PI-SceI, and between I-SceI and I-PpoI. The PacI restriction endonuclease is subsequently used not only to linearize the construct after ligation and liberate the left- and right ITRs, but also to eliminate non-recombinants.

FIG. 34N: Percentage of wells showing CPE formation after transfection of PER.C6/E2A cells with pCLIP-LacZ and the adapter plasmid pIPspAdapt2.

FIG. 35: Percentage of virus producing wells (CPE positive) in a 96-well plate of PER.C6/E2A cell after propagation of the freeze/thawed transfected cell lysates. Helper molecules used for cotransfection are (1) pWE/Ad.AflII-rITRsp, (2) pWE/Ad.AflII-rITRsp.dE2A, (3) pWE/Ad.AflII-rITRsp.dXba, and (4) pWE/Ad.AflII-rITR.

FIG. 36(A and B): Schematic overview of constructing an arrayed adenoviral cDNA expression library.

FIG. 37(A, B, C, and D): Comparison of cotransfections of different adapter plasmids and pWE/Ad.AflIIrITRDE2A on 384-well plates with cotransfections on 96-well plates. The percentage of virus producing wells (CPE positive wells) scored at different time points as indicated after propagation of the freeze/thawed transfected cells to new PER. C6/E2A cells 5 days after transfection (upper panel) or 7 days after transfection (lower panel) is shown.

FIG. 38: The percentage of virus producing wells (CPE positive wells) scored at different time points as indicated after changing the medium of the transfected cells 7 days after transfection (A); after no medium change (B); and after standard propagation of the freeze/thawed transfected cells to new PER. C6/E2A cells (C).

FIG. 39(A, B, and C): The percentage of virus producing cells (CPE positive) scored after propagation of the freeze/thawed transfected cells to new PER.C6/E2A cells, in three different experiments using PER. C6/E2A cells for transfections with indicated confluency at time of transfection. Cell numbers from each flask in each experiment were counted. The cells from these flasks were used to seed 96-well plates for transfection with adenoviral adapter and helper DNA molecules.

FIG. 40: The use of adenoviral expression vectors as a semi-stable expression system for assays with a delayed readout of phenotype after infection with an adenoviral expression library. Transgene used: Green Fluorescent Protein (EGFP, Clontech). A crude PER. C6/E2A production lysate is used at a multiplicity of infection (MOI) of about 500-1000.

FIG. 41: The use of polyethylenimine (PEI) for generating adenoviral vectors in miniaturized format. Transfection efficiency, virus formation (CPE), and proliferation (toxicity) are depicted.

FIG. 42: Effect of temperature PEI at time of transfections on CPE efficiency. W: Warm (room temperature) and C: Cold (4° C.).

FIG. 43: Effect of PEI transfection volume on transfection efficiencies.

FIG. 44: Washing of PER. C6/E2A cells with serum free medium before applying lipofectamine-DNA complex can be omitted.

FIG. 45: Transcriptional control of adipogenesis. Transcriptional control involves activation of several families of transcription factors. These proteins are expressed in a network in which C/EBPβ and C/EBPδ are detected first, followed by PPARγ, which in turn activates C/EBPα and a broad program of adipogenesis. C/EBPα exerts positive feedback on PPARγ to maintain the differentiated state. ADD1/SREBP1c is regulated by insulin in fat and can be activated PPARγ by inducing its expression as well as by promoting the production of endogenous PPARγ ligand. ADD1/SREBP1c also activates many genes of lipogenesis. All these factors contribute to the expression of genes that characterize the terminally differentiated phenotype (Spiegelman and Flier, (2001) Cell 104(4):531-43).

FIG. 46: Increasing the transduction efficiency of Ad5 virus by exogenous expression of the Ad5 hCAR receptor. Adenovirus infection is initiated by the formation of complexes between the globular knob domain of the adenoviral fiber protein and a host cell receptor. The fiber receptor for the Ad groups A, C, D, E and F, including Ad5, has been identified as the CAR receptor. Cells such as cell A in the figure, that do not carry the CAR receptor or express the receptor at very low basal levels are hard to infect with Ad5 viruses. One way to efficiently infect these cells is to use an Ad virus from group B or to use a fiber variant (e.g. Ad5fibC15 or Ad5fibC20) that can enter the cell through a different receptor. We usually use the second strategy. However, this approach cannot be used when one has already made expression libraries in Ad5. We therefore devised an alternative strategy where the cells are first infected with Ad5 or an Ad5 fiber variant expressing the hCAR receptor prior to infection of the cells with the Ad5 viruses of the placental PhenoSelect™ cDNA expression library. This method allows us to infect virtually every single cell line or polyclonal primary cell population.

FIG. 47: Images taken by fluorescence microscopy showing the difference in infection efficiency using adenoviruses with different fiber modifications. Human primary pre-adipocytes are seeded at 1000 cells per well in a 384 well plate and infected with adenoviruses with different fiber modifications: Ad5C01 and Ad5C20. It is clear from the pictures, taken 3 days post infection, that Ad5C20 is superior over Ad5C01 in terms of infection efficiency. (Zeiss Axiovert 25, 10× objective).

FIG. 48: Induction of lipid droplet formation in primary human pre-adipocytes. Precursor cells are co-infected with Ad5C20-hCAR containing the receptor for Ad5C01 and either empty Ad5C01 virus (A,B) or Ad5C01-PPARγ (C,D). 7 days after infection, cells are scored microscopically for formation of lipid droplets as a marker for adipocyte differentiation. Cells are stained with the lipophilic red fluorescent dye Nile Red (A,C) to better visualize lipid droplets. Images of cells are recorded using white light phase contrast microscopy (B,D) or using fluorescence microscopy (A,C).

FIG. 49: Adipocyte differentiation induced by H5-1 and H5-2/PPARγ in human primary mesenchymal progenitor cells. Mesenchymal stem cells are infected with placenta PhenoSelect™ cDNA library viruses in the presence of Ad5C20-hCAR virus as described in FIG. 46, together with Ad5C01-eGFP, -H5-1 or -H5-2/PPARγ. Seven days post infection formation of lipid droplets is clearly induced by H5-1 and H5-2/PPARγ, but not GFP. Arrows indicate all clusters of lipid droplets, present in the cytoplasm of the cells.

FIG. 50: Adipocyte differentiation induced by H5-1 and H5-2/PPARγ in murine C3H10T½ cells. 1000 C3H10T½ cells/well are seeded in a 384 well plate. One day after seeding, cells are co-infected using Ad5C20-hCAR and Ad5C01-GFP, -H5-1 or -H5-2/PPARγ. Lipid droplet formation is scored 7 days post infection. Lipid droplets are clearly visible in the cytoplasm of the cells because of the increased phase-contrast. Arrows indicate a few of the many lipid droplets. A clear difference between the number and the phenotypes of lipid droplet clusters, induced by H5-1 and H5-2/PPARγ can be seen.

FIG. 51: Adipocyte differentiation induced by H5-24 in mesenchymal stem cells, derived from fat tissue. No cell death can be observed upon adenoviral transduction of pre-adipocytes with H5-24 cDNA. 1000 human pre-adipocytes are seeded per well of a 384 well plate. The next day, cells are co-infected using Ad5C01-hCAR and Ad5C01-H5-24 virus. Seven days later, cells are analysed for lipid droplet formation using white light microscopy, using Nile Red stainings and fluorescence microscopy. Furthermore, from white light microscopy, it is clear that 7 days post-infection, confluent monolayers are obtained, indicating that cells had proliferated. In addition, a Hoechst 33342 staining, showing the chromatin present in the nuclei of all cells, shows no condensation of chromatin. Thus exogenous expression of H5-24, a putative inducer of apoptosis, does not induce any cell death.

FIG. 52: Nucleotide sequence of SEQ ID NO: 14.

FIG. 53: Nucleotide sequence of SEQ ID NO: 16. The bolded first 17 bases are the vector sequence upstream of the cloned cDNA including the SalI cloning site. The last ten bases, shown in bold, comprise the downstream vector sequence including the NotI cloning site.

FIG. 54: Amino acid sequence of SEQ ID NO: 15.

FIGS. 55-56: SEQ ID NOS: 17 and 18. Nucleotide sequences that are complementary to BLTR2 DNA sequence.

FIG. 57: Alignment of SEQ ID NO: 17 with DNA sequence complementary to BLTR2 sequence. SEQ ID NO: 17 is 100% identical to antisense BLTR2 DNA.

FIG. 58: Alignment of SEQ ID NO: 18 with DNA sequence complementary to BLTR2 sequence. SEQ ID NO: 18 is 100% identical to antisense BLTR2 DNA.

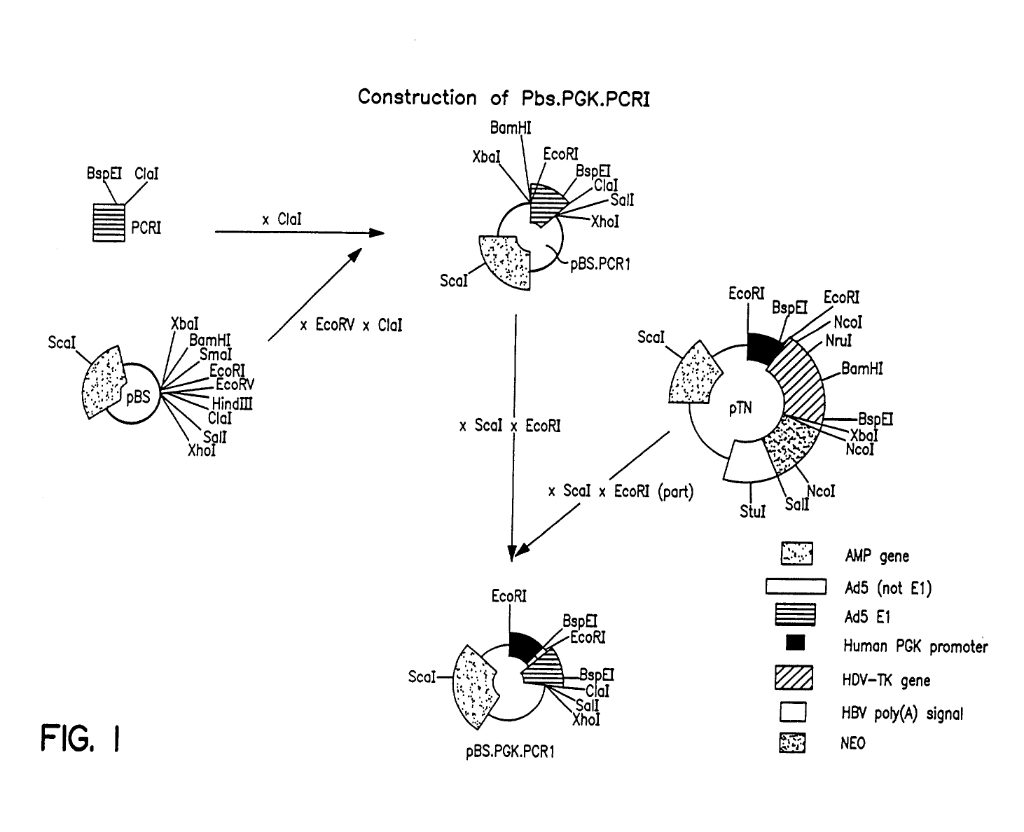

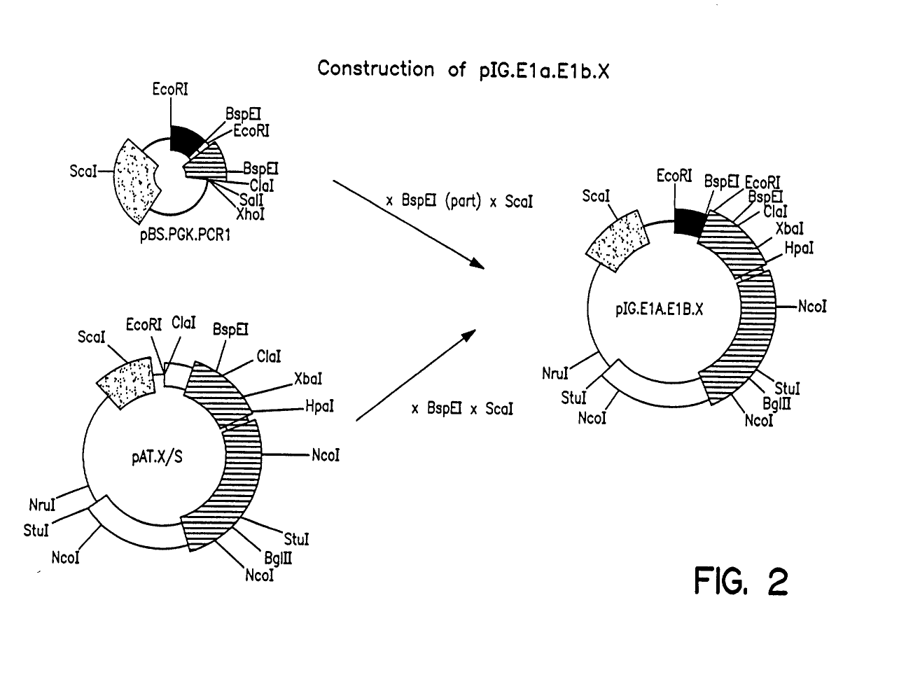

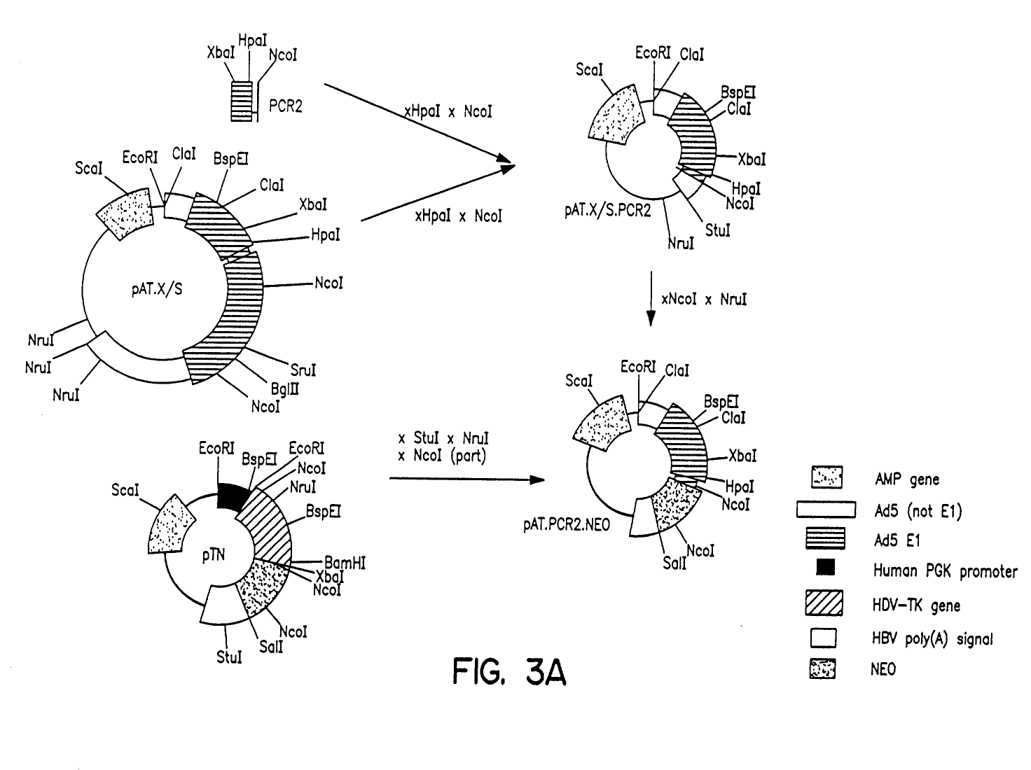

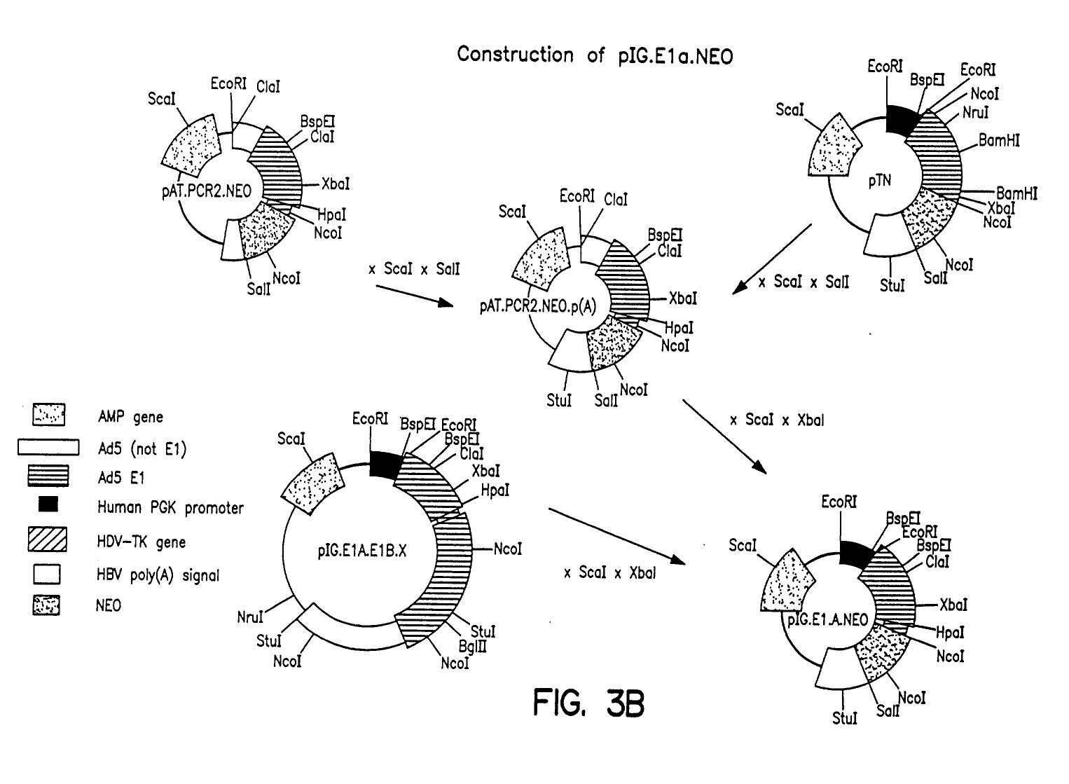

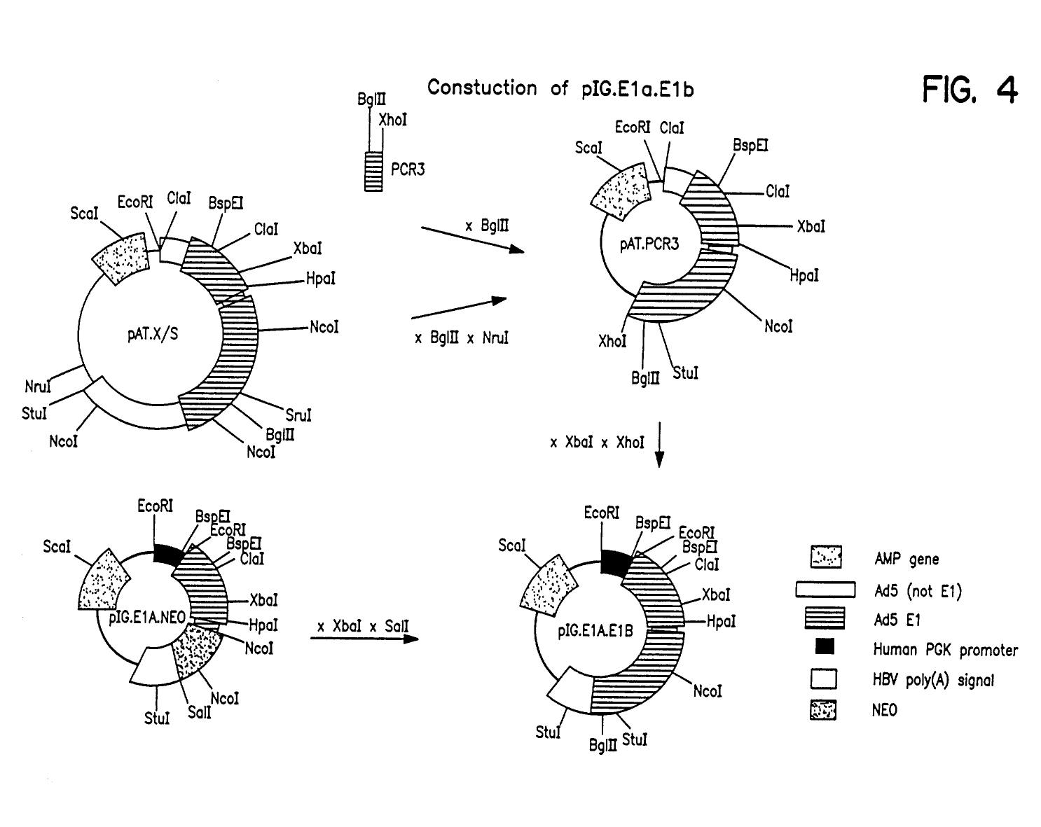

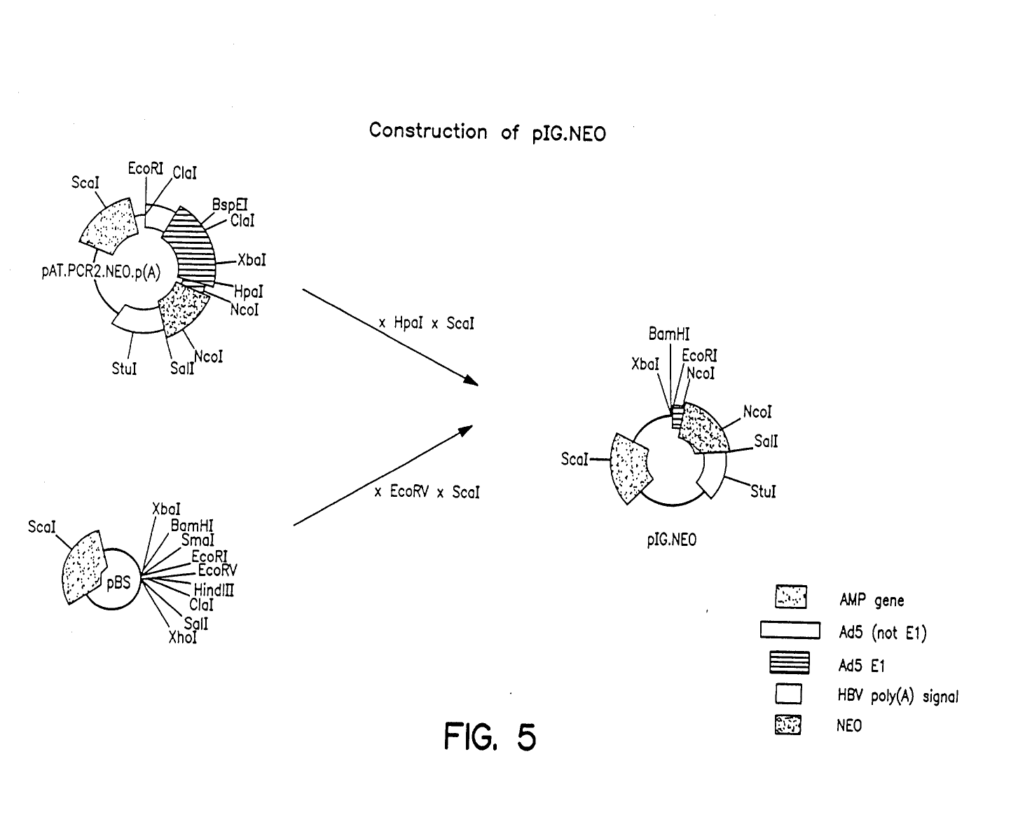

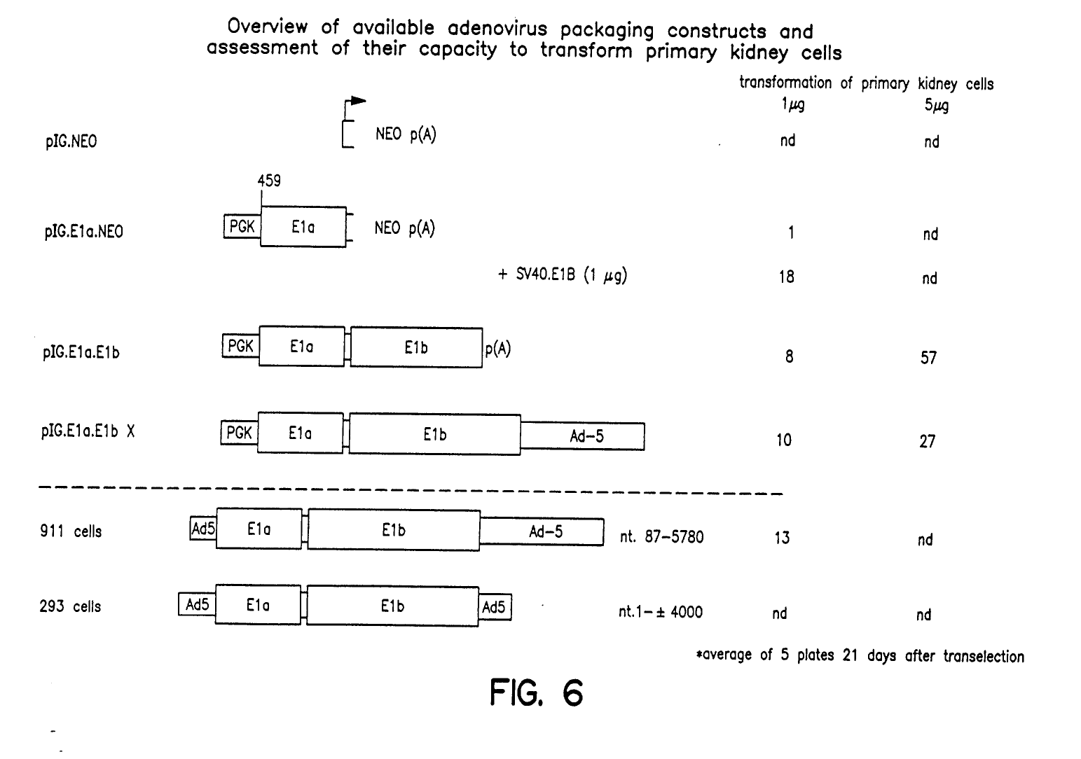

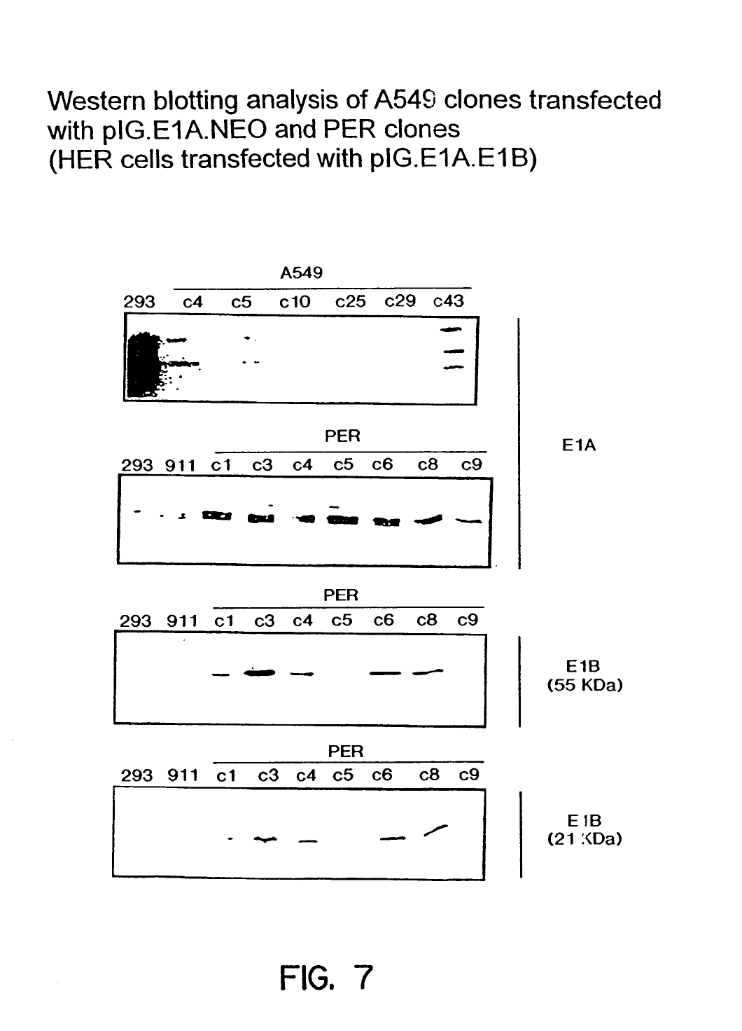

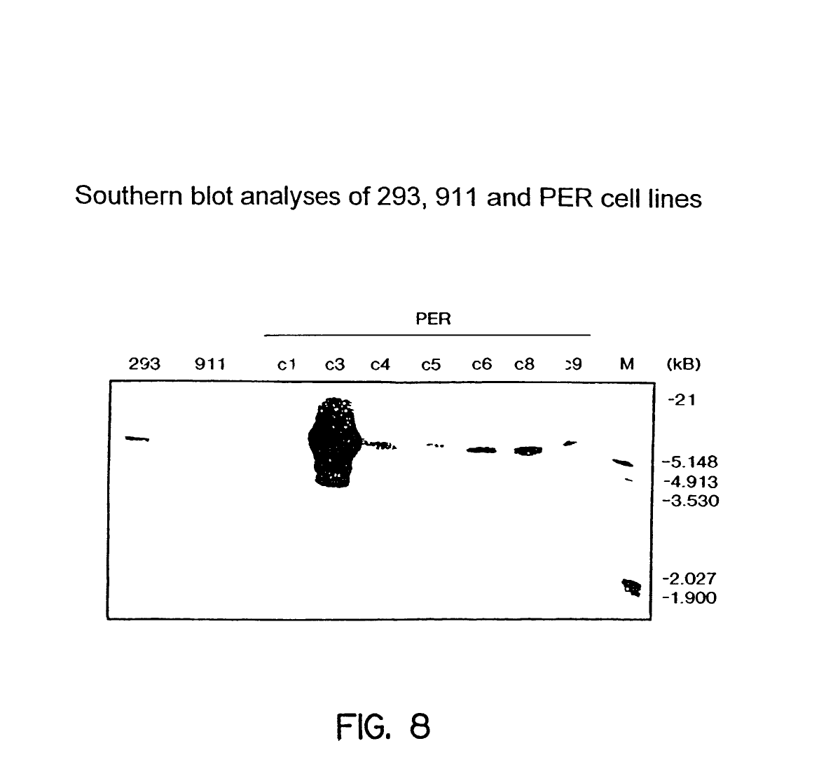

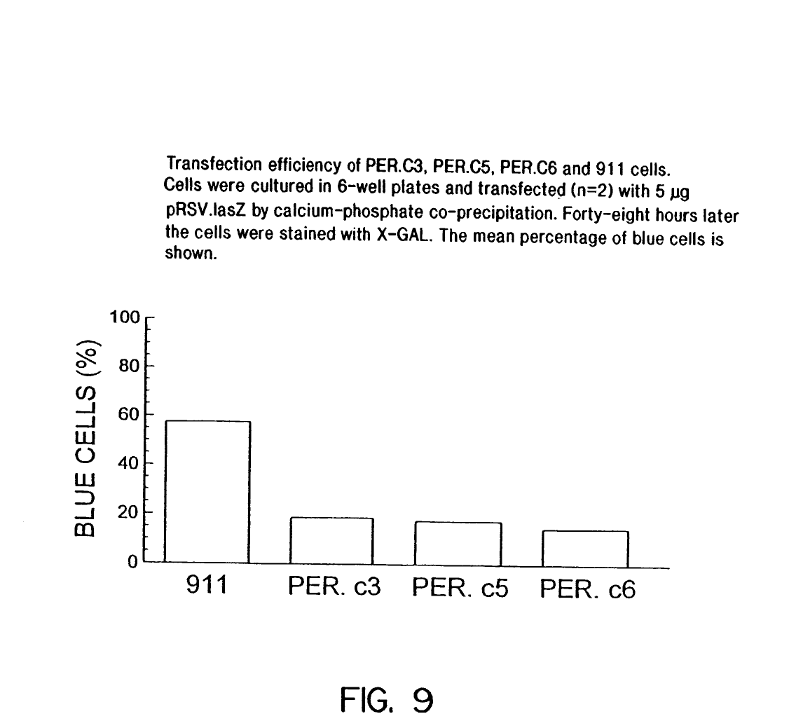

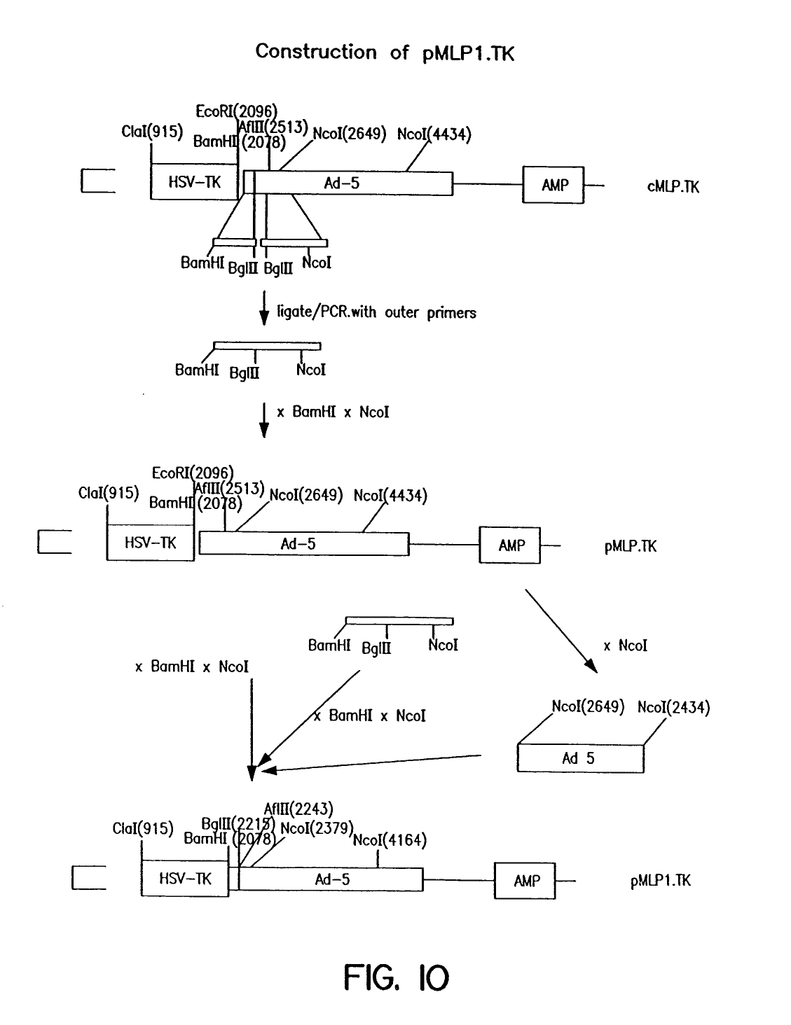

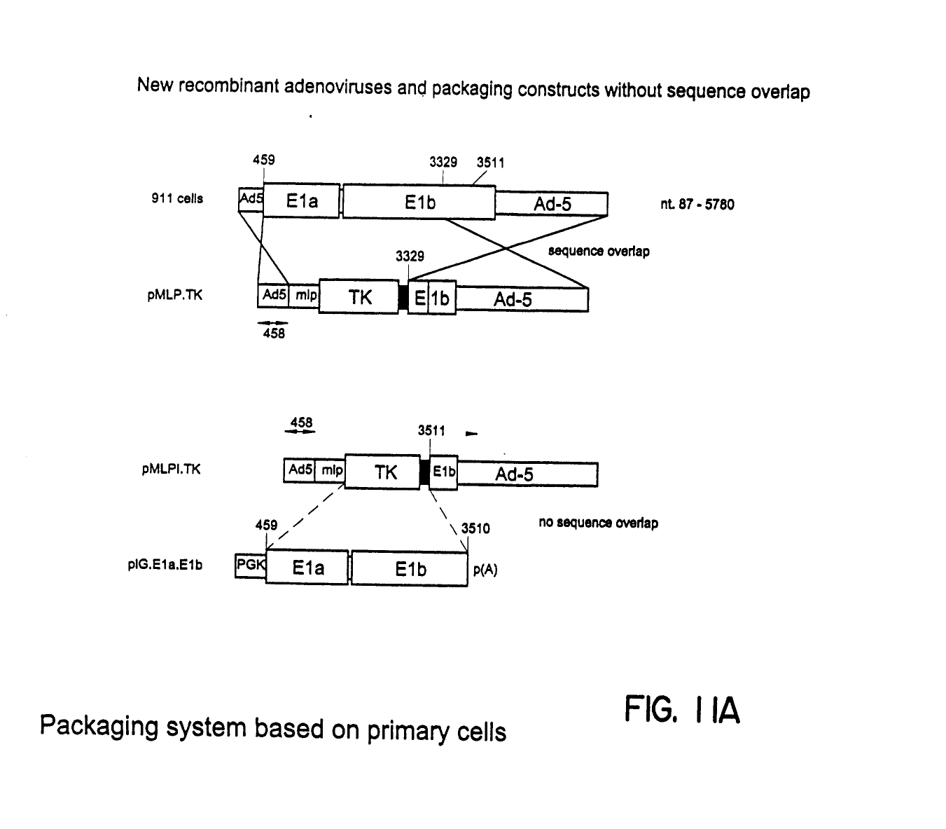

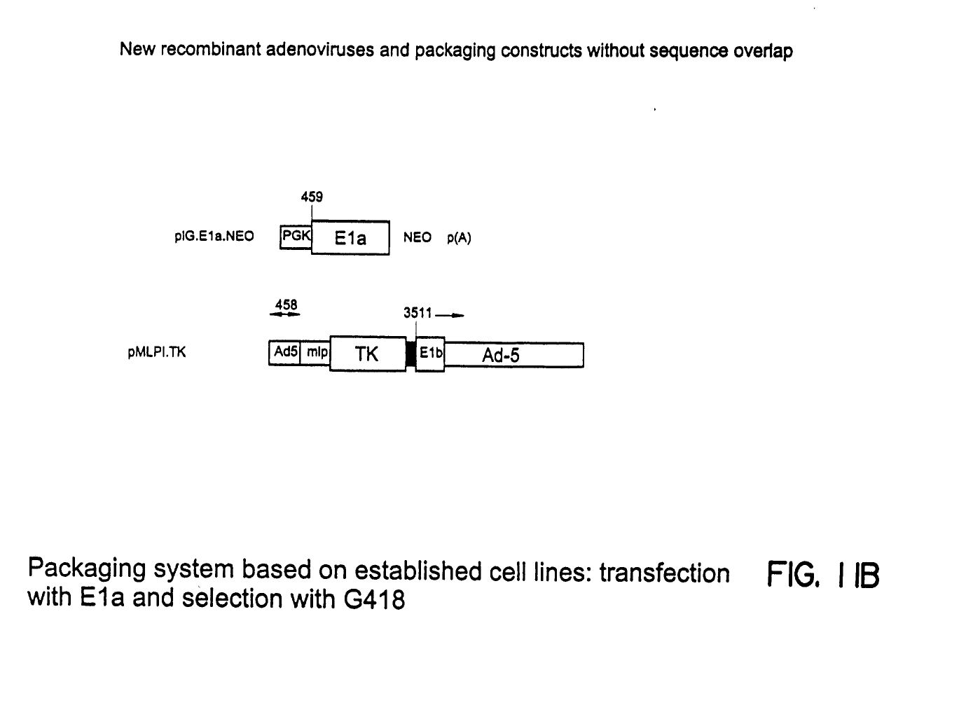

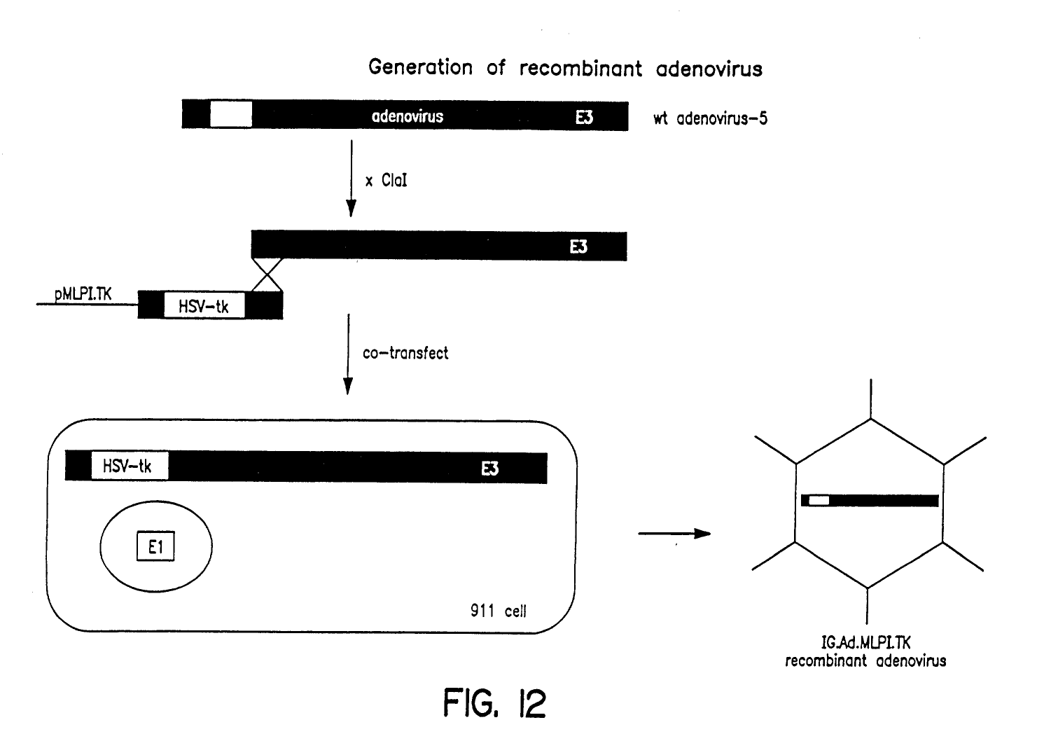

### DETAILED DESCRIPTION

The following definitions are used throughout the specification.

“Adipogenesis” (or “lipogenesis”) means the process in which a precursor cell, having the potential of becoming one or more mature cell types having committed phenotypical characteristics, otherwise known as cell differentiation, becomes an adipocyte, which is a cell characterized by the cellular function of fatty acid storage (e.g., in cytoplasmic lipid droplets). Precursor cells that are involved in the process of adipogensis include pre-adipocytes, mesenchymal stem cells and progenitor cells.

“Carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Compound” is used herein in the context of a “test compound” or a “drug candidate compound” described in connection with the screening assays of the present invention. As such, these compounds comprise organic or inorganic compounds, derived synthetically or from natural sources. The compounds include inorganic or organic compounds such as polynucleotides or hormone analogs that are characterized by relatively low molecular weights. Other biopolymeric organic test compounds include ribozymes, peptides comprising from about 2 to about 40 amino acids and larger polypeptides comprising from about 40 to about 500 amino acids, such as antibodies or antibody conjugates.

“Disease” means the overt presentation of symptoms (i.e., illness) or the manifestation of abnormal clinical indicators (e.g., biochemical indicators), resulting from defects in one or more of the metabolic processes of insulin action, glucose metabolism or uptake, fatty acid metabolism or uptake or catecolamine action. Alternatively, the term “disease” refers to a genetic or environmental risk of- or propensity for developing such symptoms or abnormal clinical indicators. Diseases associated with defects in insulin action and fatty acid metabolism or uptake include, but are not limited to, the common insulin resistance syndromes including, but not limited to, metabolic syndrome, syndrome X. Diseases associated with insulin action include, but are not limited to, non-insulin-dependent diabetes (NIDDM), combined hyperlipidemia (including, but not limited to, familial combined hyperlipidemia) and essential hypertension.

“Expressible nucleic acid” means a nucleic acid coding for a proteinaceous molecule, an RNA molecule, or a DNA molecule.

“Hybridization” refers to any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, stringency can be increased by reducing the concentration of salt, increasing the concentration of formamide, or raising the hybridization temperature.

“Hypertension” means an elevation in resting blood pressure of at least 10% relative to that of normal individuals of comparable age, height and weight.

“Mammal” means any animal classified as a mammal, including humans, domestic and farm animals, and zoo, sports, or pet animals, such as dogs, horses, cats, hamsters, rats, mice, cattle pigs, goats, sheep, etc.

“Metabolic Syndrome” or otherwise known as “Syndrome X” means a disease characterized by spontaneous hypertension, dyslipidemia, insulin resistance, hyperinsulinemia, increased abdominal fat and increased risk of coronary heart disease.

“Non-insulin-dependent diabetes” refers to type 2 diabetes, which is characterized by insulin resistance, impaired glucose tolerance and impaired fasting glycemia.

“Obesity” refers to a condition in which the body weight of a mammal exceeds medically recommended limits by at least about 20%., based upon age and skeletal size.

“Polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common.. The polynucleotides include polyribonucleic acids, polydeoxyribonucleic acids, and synthetic analogues thereof The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs).

“Treatment” means an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. Administration “in combination with” or “admixture with” one or more further therapeutic agents includes simultaneous (concurrent) and consecutive administration in any order.

Library Screening For Adipogenesis-Related Functional Genes

The present invention, in one embodiment, provides methods that use a library of expressible nucleic acids comprising a multiplicity of compartments. Each compartment comprises at least one vehicle including at least one nucleic acid of the library, whereby the vehicle is capable of introducing at least one nucleic acid into a cell such that it can be expressed. Another advantage of the library is that it includes a multiplicity of compartments each including at least one nucleic acid. When a compartment includes only one nucleic acid, then it is known that the unique nucleic acid in the distinct compartment is responsible for whatever change in phenotype is observed.

In one embodiment, at least one compartment includes at least two vehicles. Especially with, but not limited to, large libraries, it becomes advantageous to reduce the number of compartments to reduce the number of screening assays that need to be performed. In such cases, libraries are provided that include more than one vehicle. If after screening, a certain effect is correlated to a certain compartment, the vehicles in the compartment may be analysed separately in an additional screening assay to select the vehicle including the nucleic acid the expression of which exerts the effect. In addition, the presence of more than one vehicle in a compartment may be advantageous when a library containing one vehicle per compartment is screened for a nucleic acid capable of exerting an effect in combination with one particular other nucleic acid. The other nucleic acid may then be provided to the cell by adding a vehicle including the particular other nucleic acid to all compartments prior to performing the screening assay. Similarly, the vehicle may include at least two nucleic acids.

The library used in the method may use any kind of cell. Preferably, when the library is screened for the presence of nucleic acids with potential therapeutic values, the cell is a eukaryotic cell, especially a mammalian cell. In a preferred embodiment, the cells are divided over a number of compartments each including at least one vehicle including at least one nucleic acid from the library. The number of compartments preferably corresponds to the multiplicity of compartments in the library.

In a preferred embodiment, the vehicle includes a viral element or a functional part, derivative and/or analogue thereof. A viral element may include a virus particle such as, but not limited to, an enveloped retrovirus particle or a virus capsid of a non-enveloped virus such as, but not limited to, an adenovirus. A virus particle is favorable since it allows the efficient introduction of at least one nucleic acid into a cell. A viral element may also include a viral nucleic acid allowing the amplification of the library in cells. A viral element may include a viral nucleic acid allowing the packaging of at least one nucleic acid into a vehicle, where the vehicle is a virus particle. In a preferred embodiment, the viral element is derived from an adenovirus. Preferably, the vehicle includes an adenoviral vector packaged into an adenoviral capsid, or a functional part, derivative, and/or analogue thereof Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses.

In a preferred embodiment, the nucleic acid derived from an adenovirus includes the nucleic acid encoding an adenoviral late protein or a functional part, derivative, and/or analogue thereof An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof Preferably, the nucleic acid derived from an adenovirus includes the nucleic acid encoding adenovirus E2A or a functional part, derivative, and/or analogue thereof Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell.

In one embodiment, the nucleic acid derived from an adenovirus includes the nucleic acid encoding at least one E1-region protein or a functional part, derivative, and/or analogue thereof The presence of the adenoviral nucleic acid encoding an E1-region protein facilitates, at least in part, replication of the nucleic acid in a cell. The replication capacity is favored in certain applications when screening is done for expressible nucleic acids capable of irradiating tumor cells. In such cases, replication of an adenoviral nucleic acid leading to the amplification of the vehicle in a mammal including tumor cells may lead to the irradiation of metastasised tumor cells. On the other hand, the presence of an adenoviral nucleic acid encoding an E1-region protein may facilitate, at least in part, amplification of the nucleic acid in a cell for the amplification of vehicles including the adenoviral nucleic acid. In one embodiment, the vehicle further includes a nucleic acid including an adeno-associated virus terminal repeat or a functional part, derivative, and/or analogue thereof which allows the integration of at least one nucleic acid in a cell.

The present invention provides a method for identifying adipogenesis-related functions of the unique nucleic acids present in a library, the functions of which are for the most part unknown, or at least not completely understood. This method transduces a multiple subpopulations of cells, each subpopulation present in a discrete compartment of the library, with at least one vehicle including at least one nucleic acid from the library, culturing the cells while allowing for expression of the nucleic acid, and determining the expressed function. The library is screened for the presence of expressible nucleic acids capable of influencing, at least in part, the formation of lipid vacuoles or the process of adipogenesis.

The present method preferably utilizes a set of adapter plasmids by inserting a set of cDNAs, DNAs, ESTs, genes, synthetic oligonucleotides, or a library of nucleic acids into E1-deleted adapter plasmids; cotransfecting an E1-complementing cell line with the set or library of adapter plasmids and at least one plasmid having sequences homologous to sequences in the set of adapter plasmids and which also includes all adenoviral genes not provided by the complementing cell line or adapter plasmids necessary for replication and packaging to produce a set or library of recombinant adenoviral vectors preferably in a miniaturized, high throughput setting. The plasmid-based system is used to rapidly produce adenoviral vector libraries that are preferably replications competent adenovirus (“RCA”)-free for high throughput screening. Each step of the method can be performed in a multiwell format and automated to further increase the capacity of the system. This high throughput system facilitates expression analysis of a large number of sample nucleic acids from human and other organisms both in vitro and in vivo and is a significant improvement over other available techniques in the field.

The method permits the amplification of the vehicles including the unique nucleic acids present in a library. Such amplification may be achieved culturing the cell with the vehicle, allowing the amplification of the vehicle, and harvesting vehicles amplified by the cell. Preferably, the cell is a primate cell thereby enabling the amplification of vehicles including viral elements that allow replication of the vehicle nucleic acid. Preferably, the cell includes a nucleic acid encoding an adenoviral E1-region protein thereby allowing, among other things, the amplification of vehicles including viral elements derived from adenovirus including adenoviral nucleic acids including a functional deletion of at least part of the E1-region. Preferably, the cell is a PER.C6 cell (ECACC deposit number 96022940) or a functional derivative and/or analogue thereof A PER.C6 cell (or a functional derivative and/or analogue thereof) allows the replication of adenoviral nucleic acid with a deletion of the E1-coding region without concomitant production of RCA in instances wherein the adenoviral nucleic acid and chromosomal nucleic acid in the PER. C6 cell or functional derivative and/or analogue thereof do not include sequence overlap that allows for homologous recombination between the adenoviral and chromosomal nucleic acid leading to the formation of RCA. Preferably, the cell further includes nucleic acid encoding adenovirus E2A and/or an adenoviral E4-region protein or a functional part, derivative, and/or analogue thereof This allows the replication of adenoviral nucleic acid with functional deletions of nucleic acid encoding adenovirus E2A and/or an adenoviral E4-region protein, thereby inhibiting replication of the adenoviral nucleic acid in a cell not including nucleic acid encoding adenovirus E2A and/or an adenoviral E4-region protein or a functional part, derivative and/or analogue thereof, for instance a cell capable of displaying a certain function.

In a preferred method, the vehicle nucleic acid does not include sequence overlap with other nucleic acids present in the cell, leading to the formation of vehicle nucleic acid capable of replicating in the absence of E1-region encoded proteins.

The method is preferably implemented using a multiplicity of compartments in a multiwell format. A multiwell format is very suited for automated execution of at least part of the methods of the invention.

The present invention uses high throughput generation of recombinant adenoviral vector libraries containing one or more sample nucleic acids, followed by high throughput screening of the adenoviral vector libraries in a host to alter the phenotype of the host as a means of assigning a function to expression product(s) of the sample nucleic acids. Libraries of E1-deleted adenoviruses are generated in a high throughput setting using nucleic acid constructs and transcomplementary packaging cells. The sample nucleic acid libraries can be a set of distinct defined or undefined sequences or can be a pool of undefined or defined sequences. The first nucleic acid construct is a relatively small and easy to manipulate adapter plasmid containing, in an operable configuration, at least a left ITR, a packaging signal, and an expression cassette with the sample nucleic acids. The second nucleic acid construct contains one or more nucleic acid molecules that partially overlap with each other and/or with sequences in the first construct. The second construct also contains at least all adenovirus sequences necessary for replication and packaging of a recombinant adenovirus not provided by the adapter plasmid or packaging cells. The second nucleic acid construct is deleted in E1-region sequences and optionally E2B region sequences other than those required for virus generation and/or E2A, E3 and/or E4 region sequences. Cotransfection of the first and second nucleic acid constructs into the packaging cells leads to homologous recombination between overlapping sequences in the first and second nucleic acid constructs and among the second nucleic acid constructs when it is made up of more than one nucleic acid molecule. Generally, the overlapping sequences are no more than 5000 bp and encompass E2B region sequences essential for virus production. Recombinant viral DNA is generated with an E1-deletion that is able to replicate and propagate in the E1-complementing packaging cells to produce a recombinant adenoviral vector library. The adenoviral vector library is introduced in a high throughput setting into a host which is grown to allow sufficient expression of the product(s) encoded by the sample nucleic acids to permit detection and analysis of its biological activity. The host can be cultured cells in vitro or an animal or plant model. Sufficient expression of the product(s) encoded by the sample nucleic acids alters the phenotype of the host. Using any of a variety of in vitro and/or in vivo assays for biological activity, the altered phenotype is analyzed and identified and a function is thereby assigned to the product(s) of the sample nucleic acids. The plasmid-based adenoviral vector systems described here provide for the creation of large gene-transfer libraries that can be used to screen for novel genes applicable to human diseases, such as those discussed in more detail herein. Identification of a useful or beneficial biological effect of a particular adenoviral mediated transduction can result in a useful gene therapeutic product or a target for a small molecule drug for treatment of such human diseases.

There are several advantages to the library used in the present invention over currently available techniques. The entire process lends itself to automation especially when implemented in a 96-well or other multi-well format. The high throughput screening, using a number of different in vitro assays, provides a means of efficiently obtaining functional information in a relatively short period of time. The member(s) of the recombinant adenoviral libraries that exhibit or induce a desired phenotype in a host in vitro or in situ are identified to reduce the libraries to a manageable number of recombinant adenoviral vectors or clones which can be tested in vitro in an animal model.

Another distinct advantage of the present library is that the adenoviral libraries produced are capable of being RCA-free. RCA contamination throughout the libraries could become a major obstacle, especially if libraries are continuously amplified for use in multiple screening programs. A further advantage of the subject invention is minimization of the number of steps involved in the process. The methods of the subject invention do not require cloning of each individual adenovirus before use in a high throughput-screening program. Other systems include one or more steps in E. coli to achieve homologous recombination for the various adenoviral plasmids necessary for vector generation (Chartier, et al, (1996) J. Virol. 70(7):4805-10; Crouzet, et al. (1997) Proc. Natl. Acad. Sci. USA 94(4): 1414-9; He, et al. (1998) Proc. Natl. Acad. Sci. USA 95(5):2509-14). Another plasmid system that has been used for adenoviral recombination and adenoviral vector generation, and which is based on homologous recombination in human cells, is the pBHG series of plasmids. However, if this plasmid is used in 293 cells, the plasmid can become unstable because the plasmid pBHG contains two ITRs close together and also can overlap with E1 sequences. All these features are undesirable and lead to RCA production or otherwise erroneous adenoviral vector production (Bett, et al. (1994) Proc. Natl. Acad. Sci. USA 91(19):8802-6). The recombinant nucleic acids of the subject invention have been designed to avoid constructions with these undesirable features.

A further advantage of the adenoviral library is the ability of recombinant adenoviruses to efficiently transfer and express recombinant genes in a variety of mammalian cells and tissues in vitro and in vivo, resulting in the high expression of the transferred sample nucleic acids. The ability to productively infect quiescent cells, further expands the utility of the recombinant adenoviral libraries. In addition, high expression levels ensure that the product(s) of the sample nucleic acids will be expressed to sufficient levels to induce a change that can be detected in the phenotype of a host and allow the function of the product(s) encoded by the sample nucleic to be determined.

The sample nucleic acids can be genomic DNA, cDNA, previously cloned DNA, genes, ESTs, synthetic double stranded oligonucleotides, or randomized sequences derived from one or multiple related or unrelated sequences. The sample nucleic acids can also be directly expressed as polypeptides, antisense nucleic acids, or genetic suppressor elements (GSE). The sample nucleic acid sequences can be obtained from any organism including mammals (for example, man, monkey, mouse), fish (for example, zebrafish, pufferfish, salmon), nematodes (for example, C. elegans), insects (for example, Drosophila), yeasts, fungi, bacteria, and plants. Alternatively, the sample nucleic acids are prepared as synthetic oligonucleotides using commercially available DNA synthesizers and kits. The strand coding the open reading frame of the polypeptide or product of the sample nucleic acid and the complementary strand are prepared individually and annealed to form double-stranded DNA. Special annealing conditions are not required. The sequences of the sample nucleic acids can be randomized or not through mutagenizing or methodologies promoting recombination. The sample nucleic acids code for a product(s) for which a biological activity is unknown. The phrase biological activity is intended to mean an activity that is detectable or measurable either in situ, in vivo, or in vitro. Examples of a biological activity include but are not limited to altered viability, morphologic changes, apoptosis induction, DNA synthesis, tumorigenesis, disease or drug susceptibility, chemical responsiveness or secretion, and protein expression.

The sample nucleic acids preferably contain compatible ends to facilitate ligation to the vector in the correct orientation and to operatively link the sample nucleic acids to a promoter. For synthetic double-stranded oligonucleotide ligation, the ends compatible to the vector can be designed into the oligonucleotides. When the sample nucleic acid is an EST, genomic DNA, cDNA, gene, or previously cloned DNA, the compatible ends can be formed by restriction enzyme digestion or the ligation of linkers to the DNA containing the appropriate restriction enzyme sites. Alternatively, the vector can be modified by the use of linkers. The restriction enzyme sites are chosen so that transcription of the sample nucleic acid from the vector produces the desired product, i.e., polypeptide, antisense nucleic acid, or GSE.

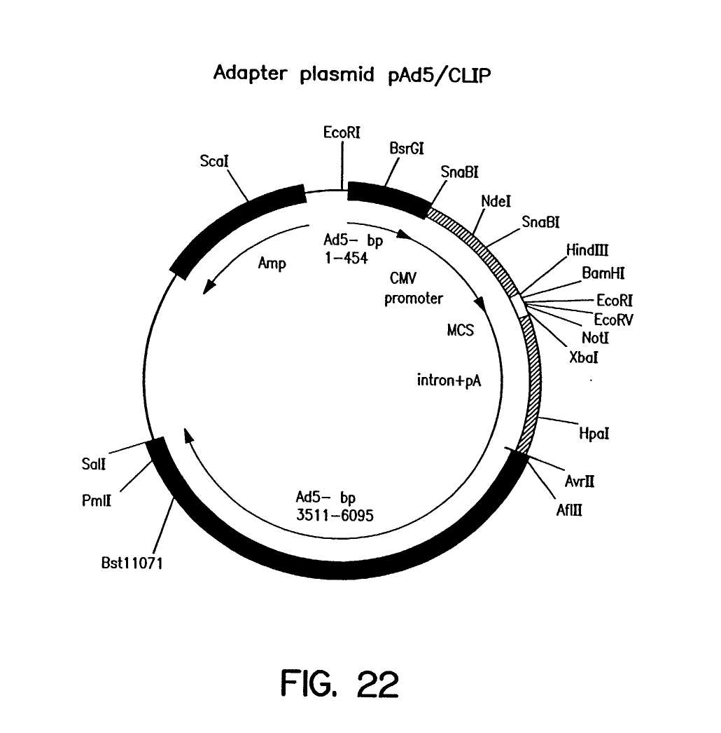

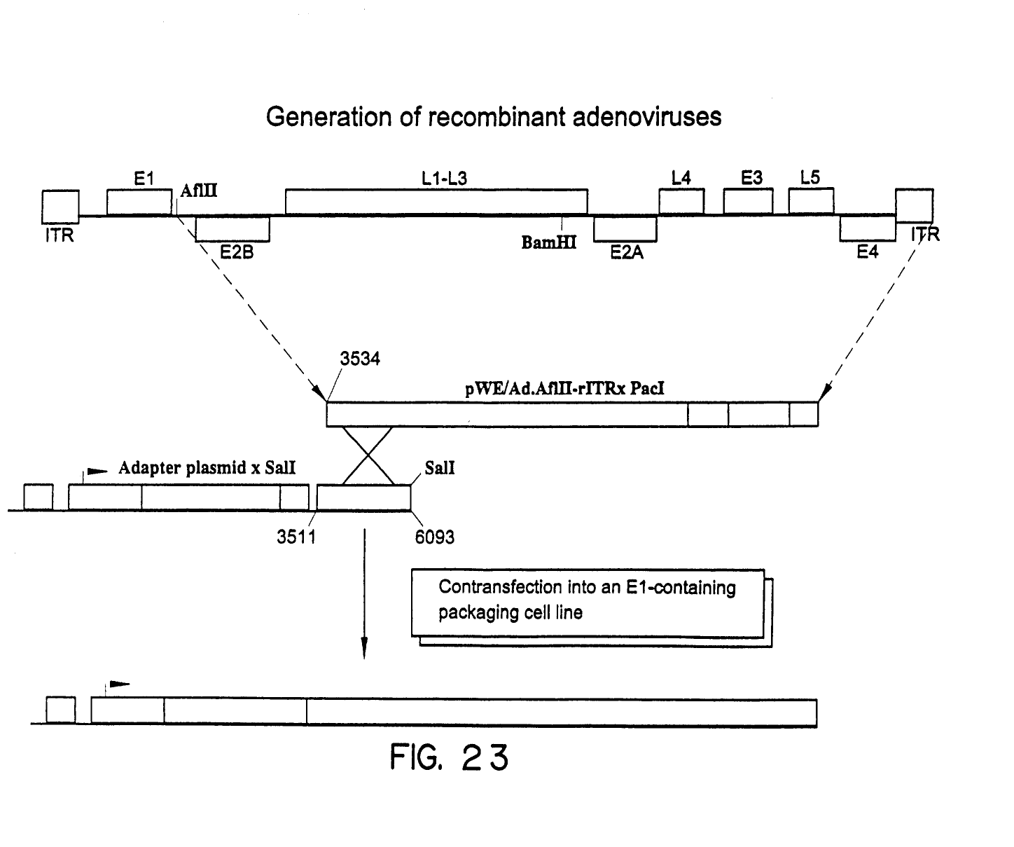

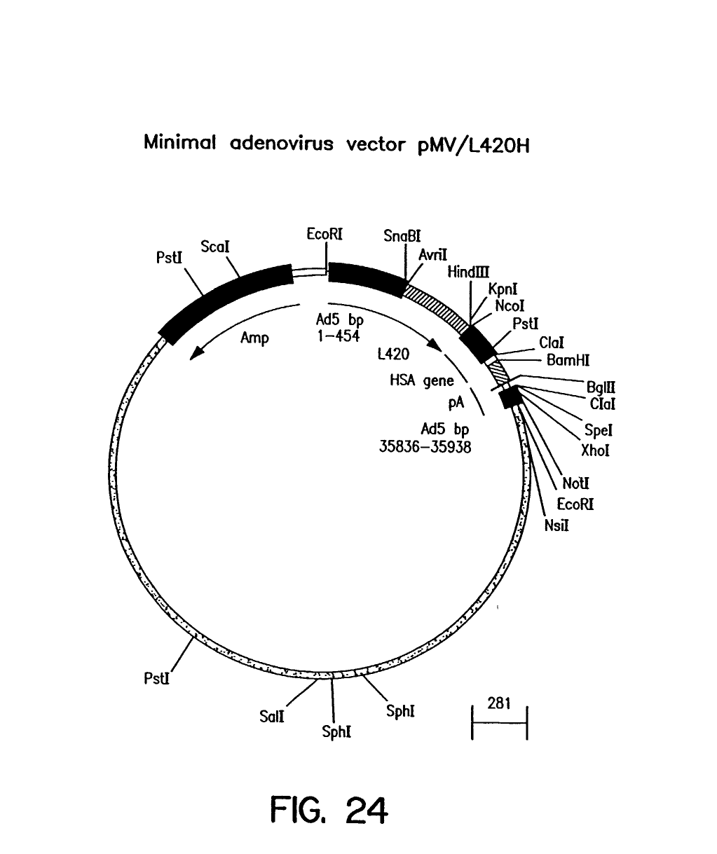

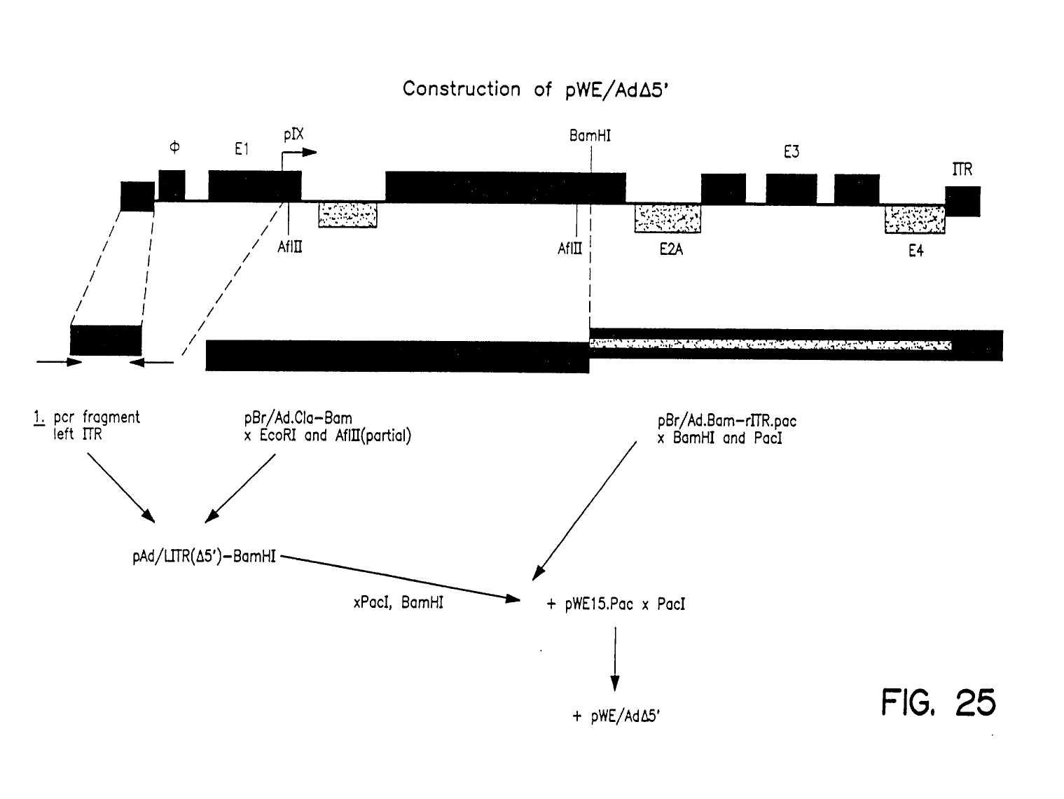

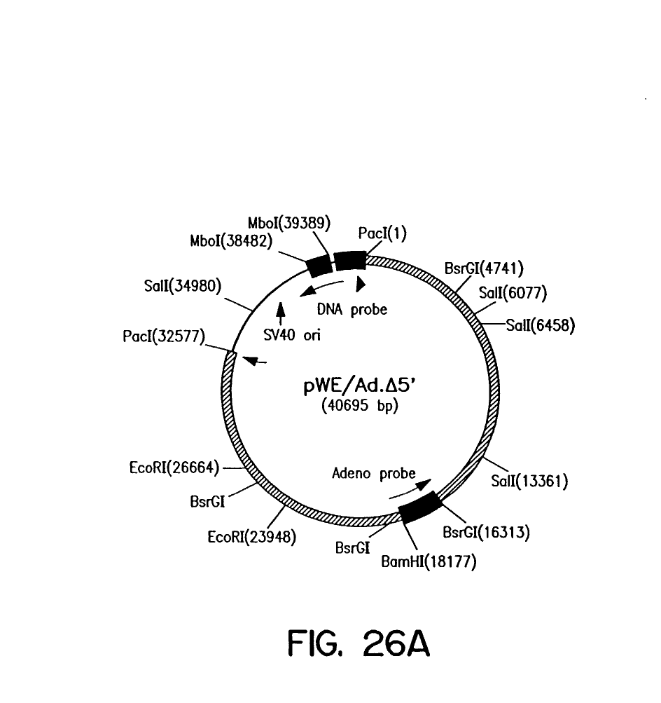

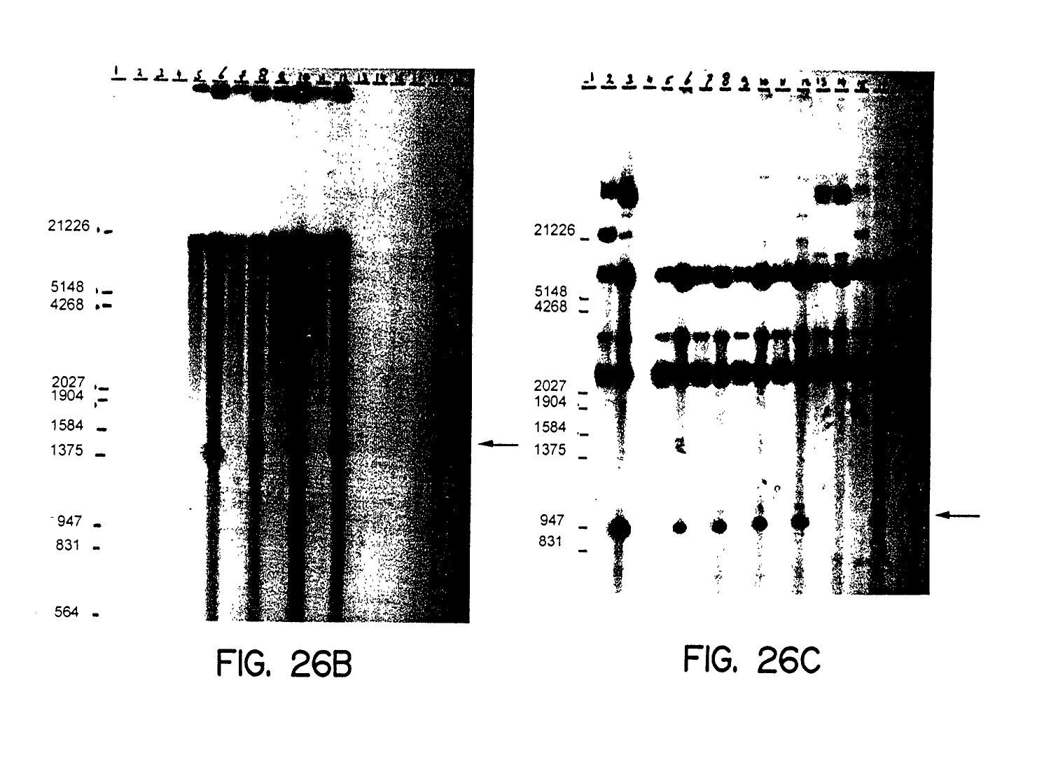

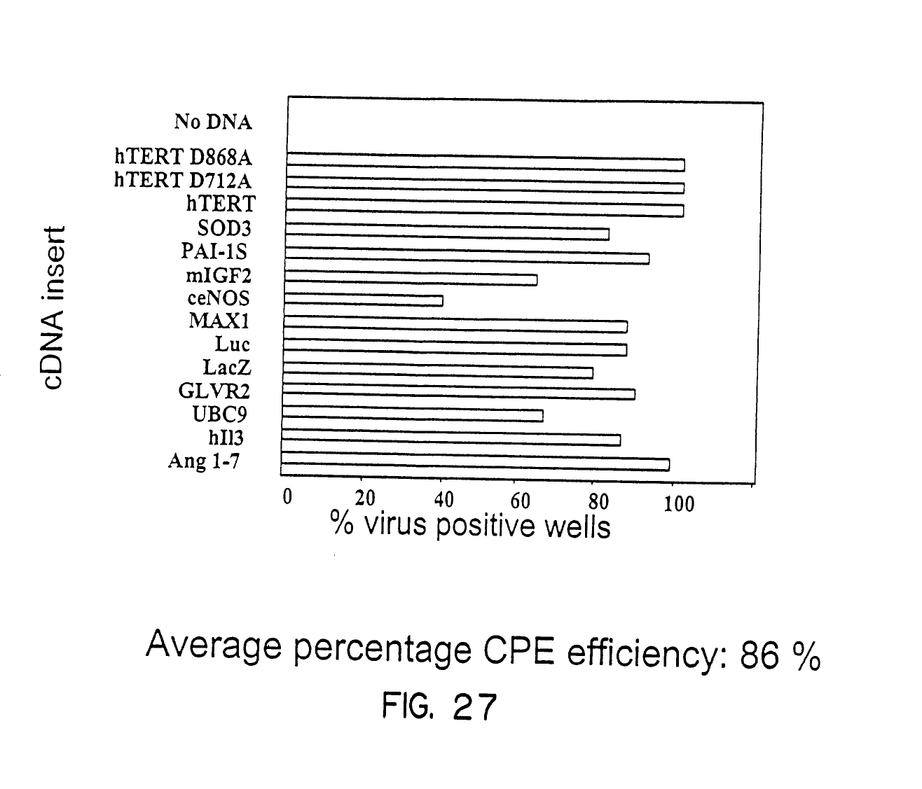

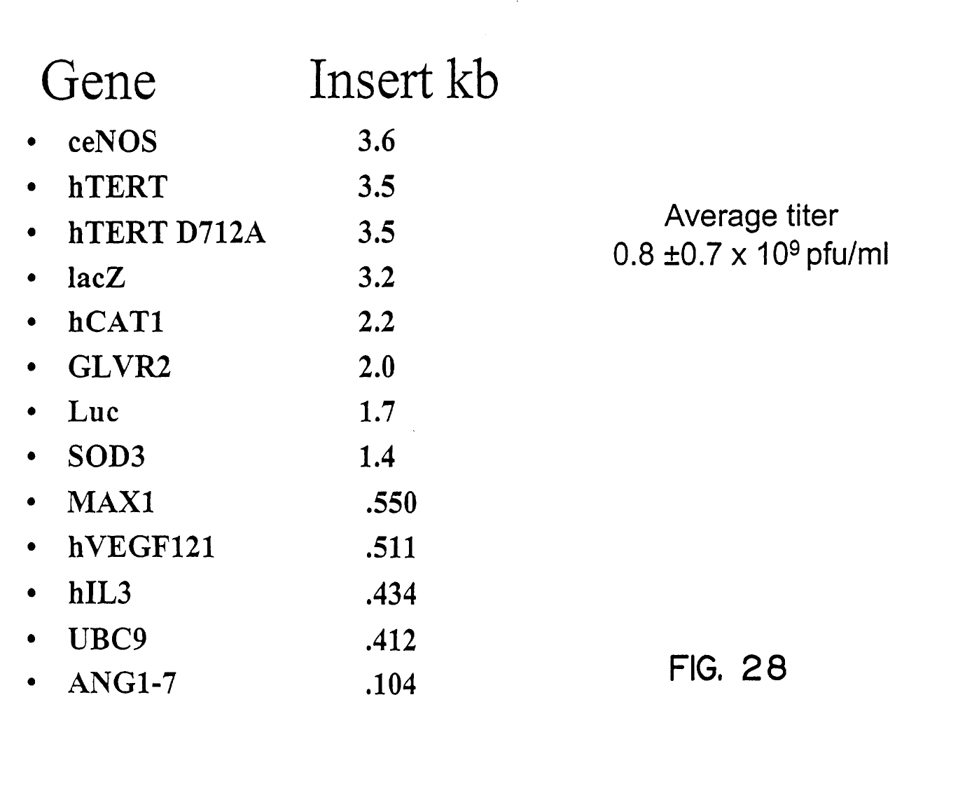

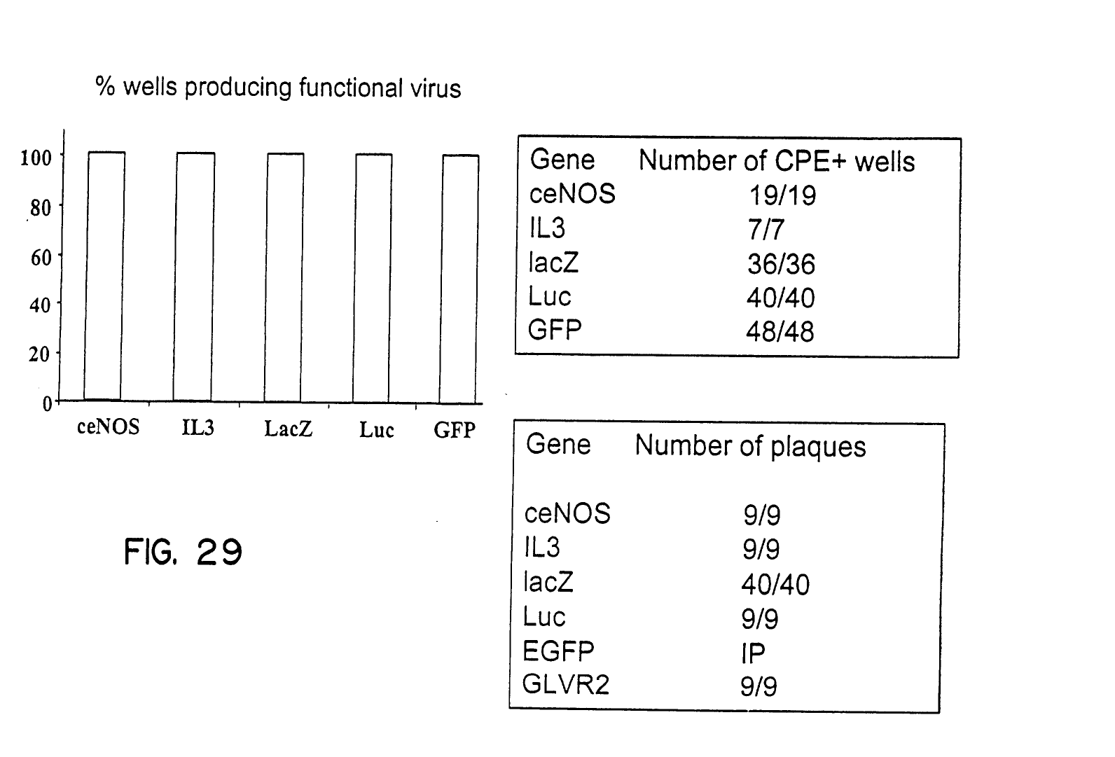

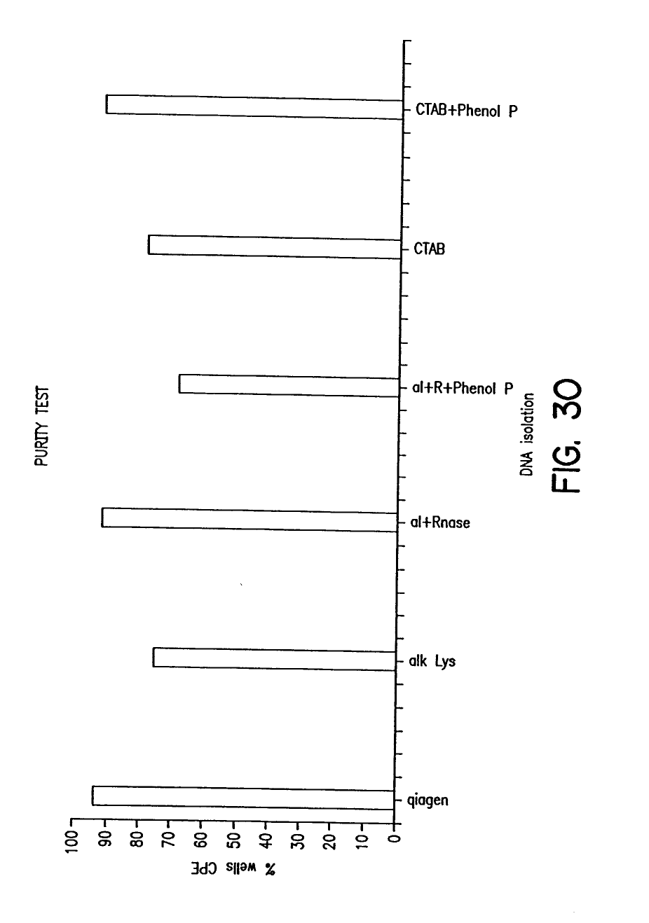

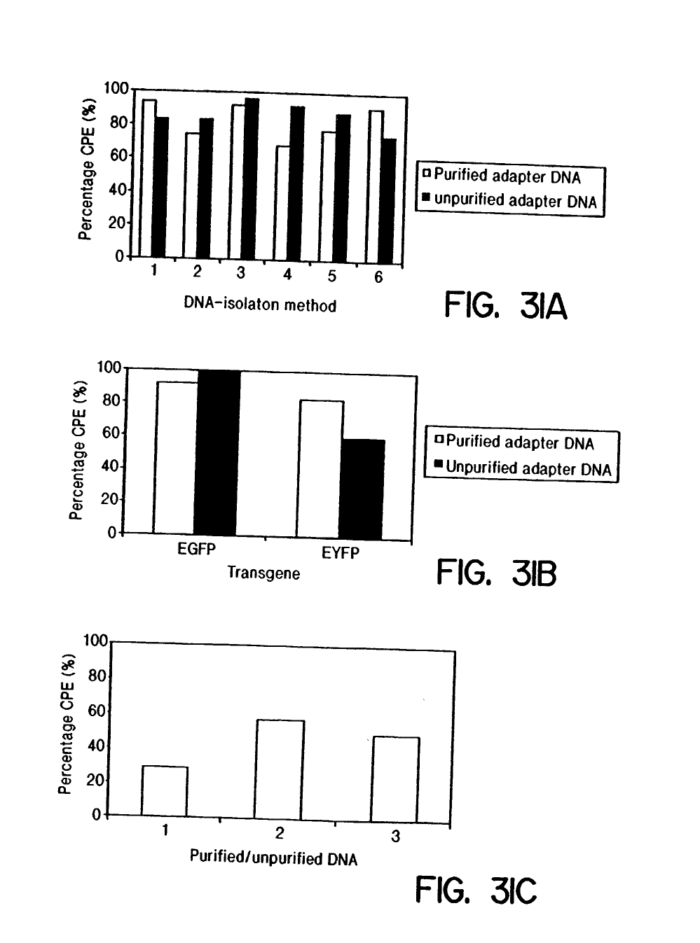

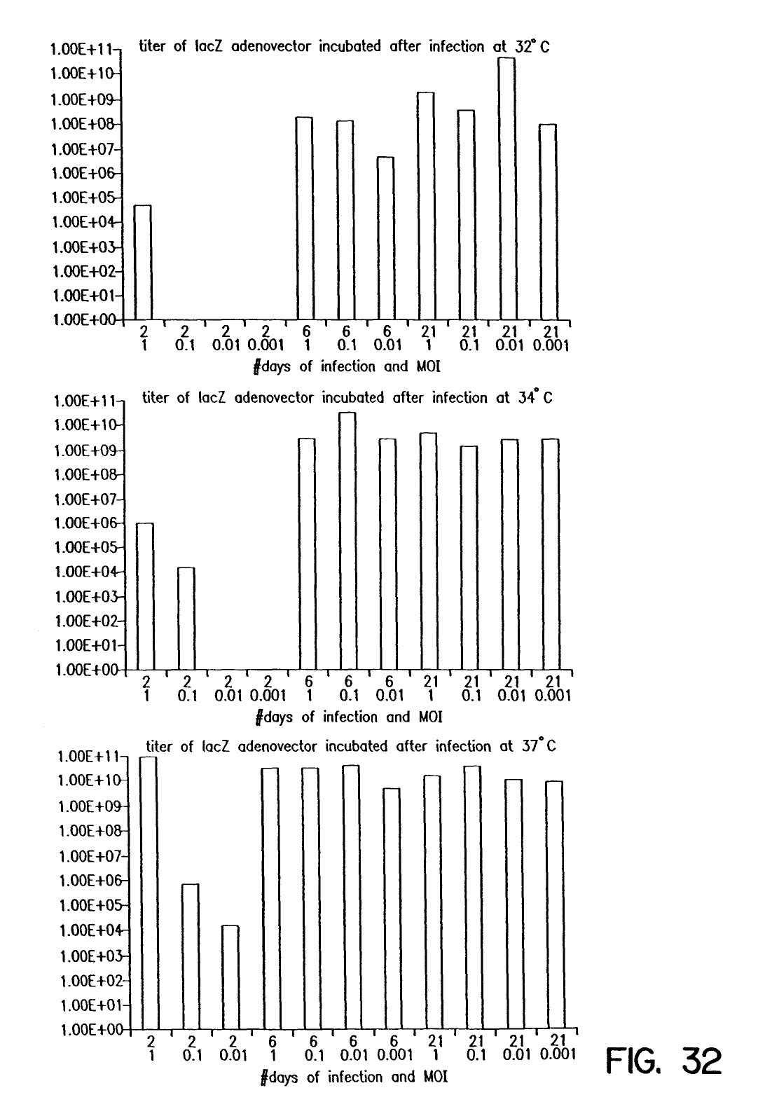

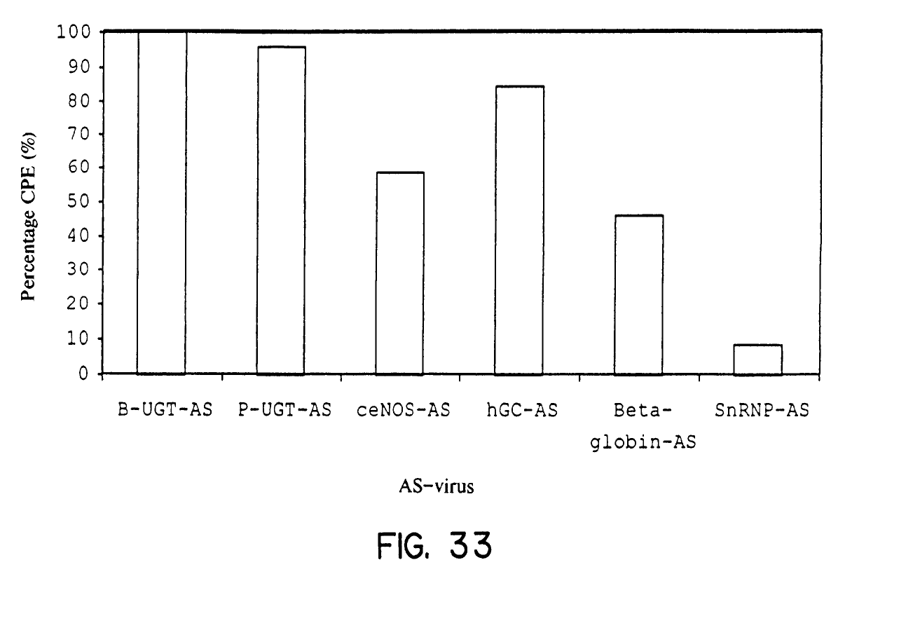

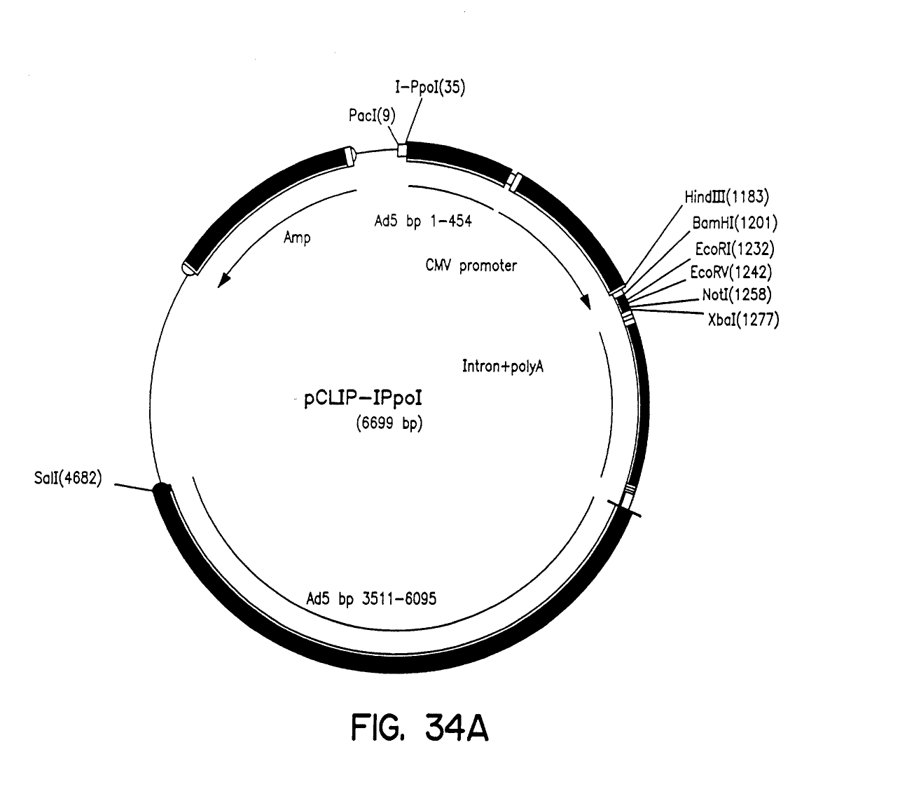

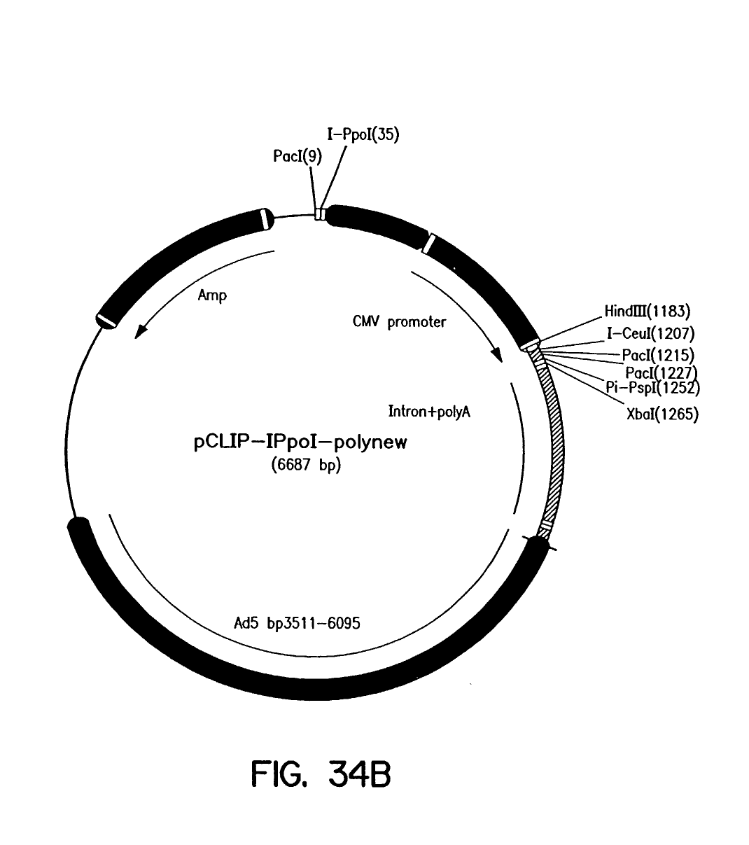

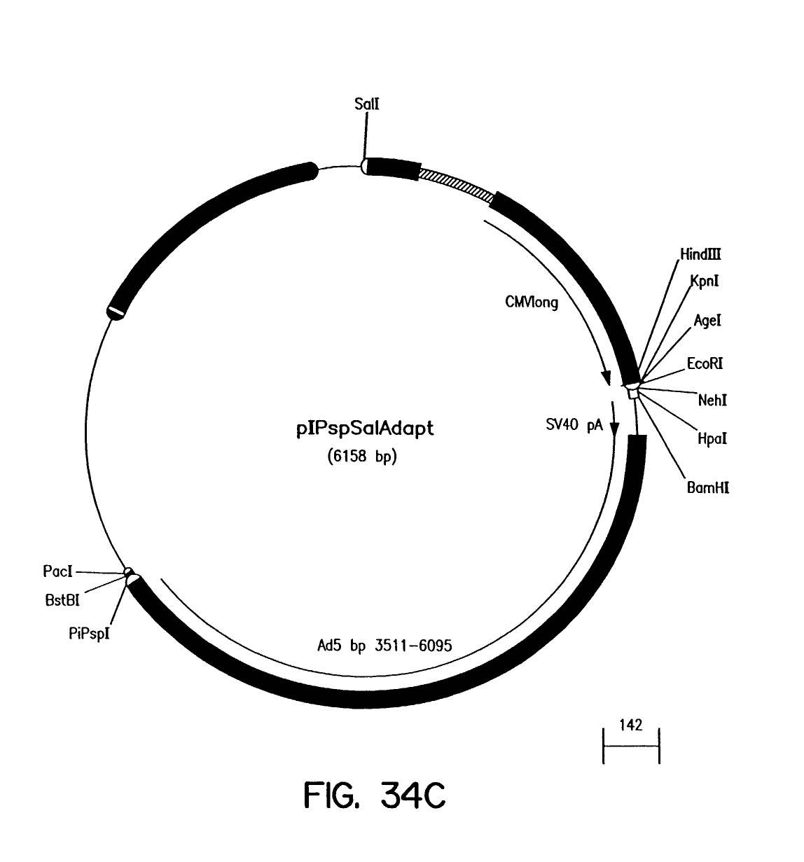

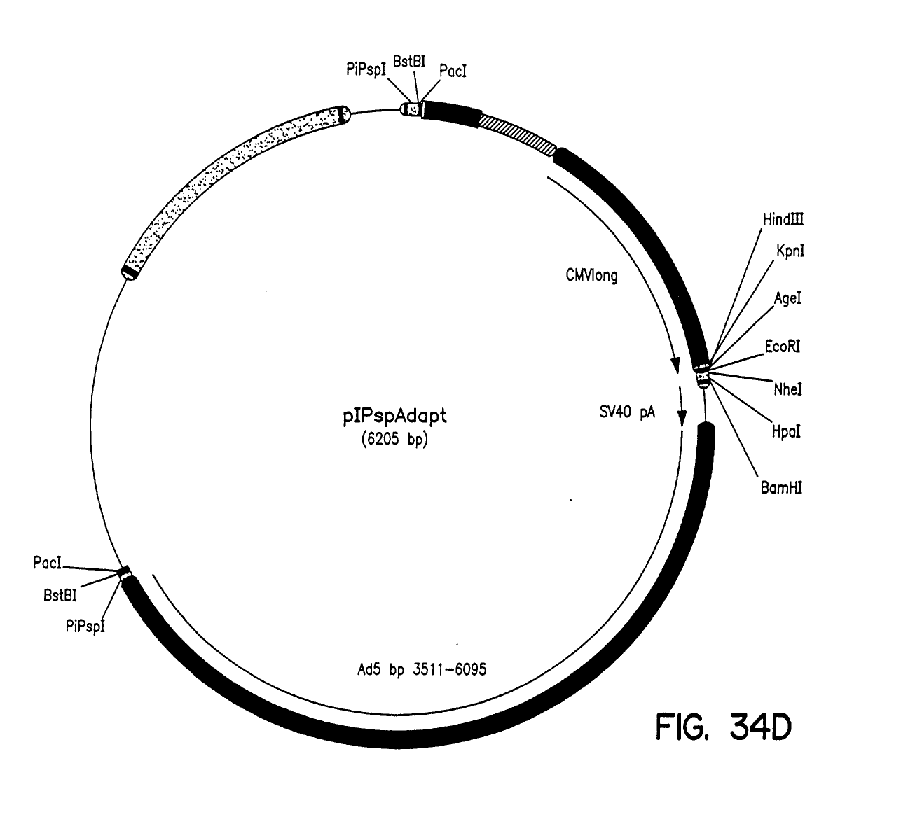

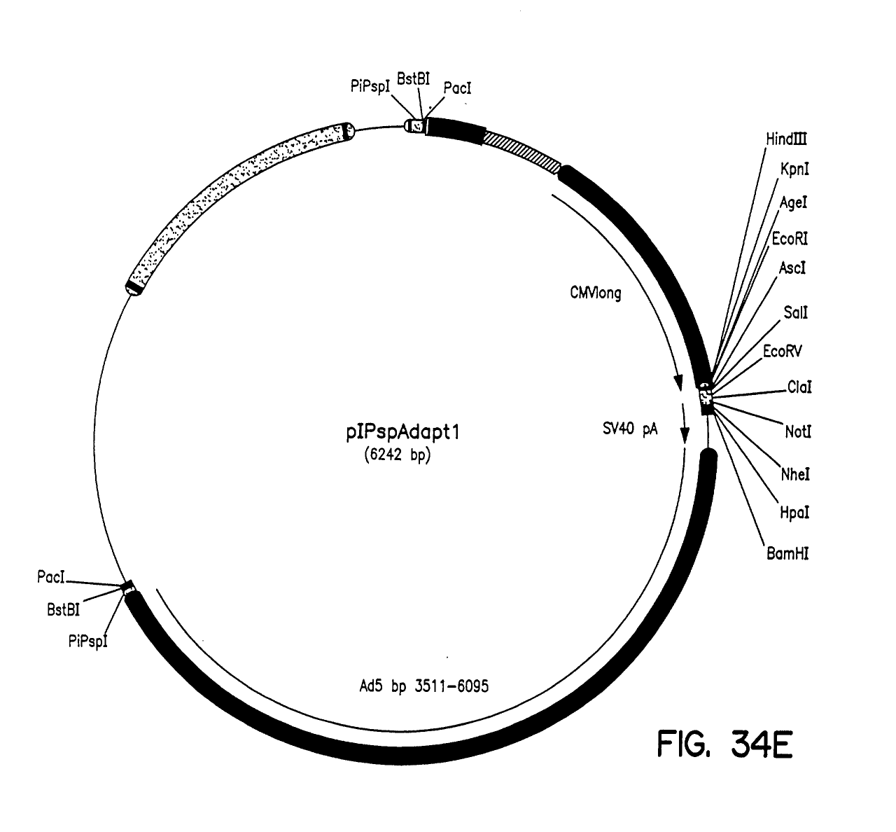

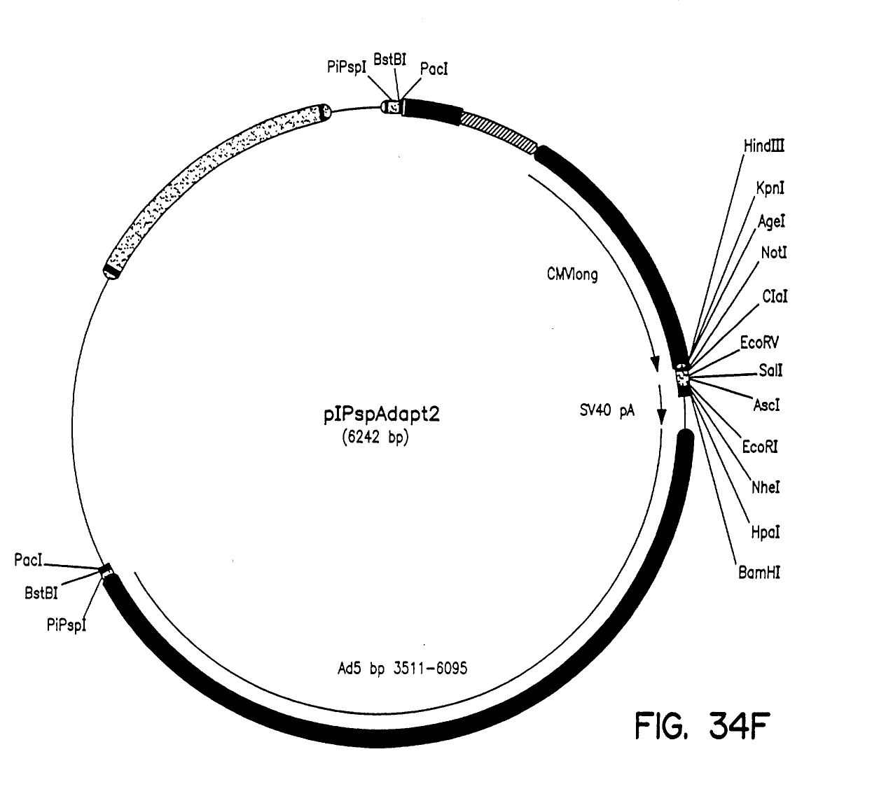

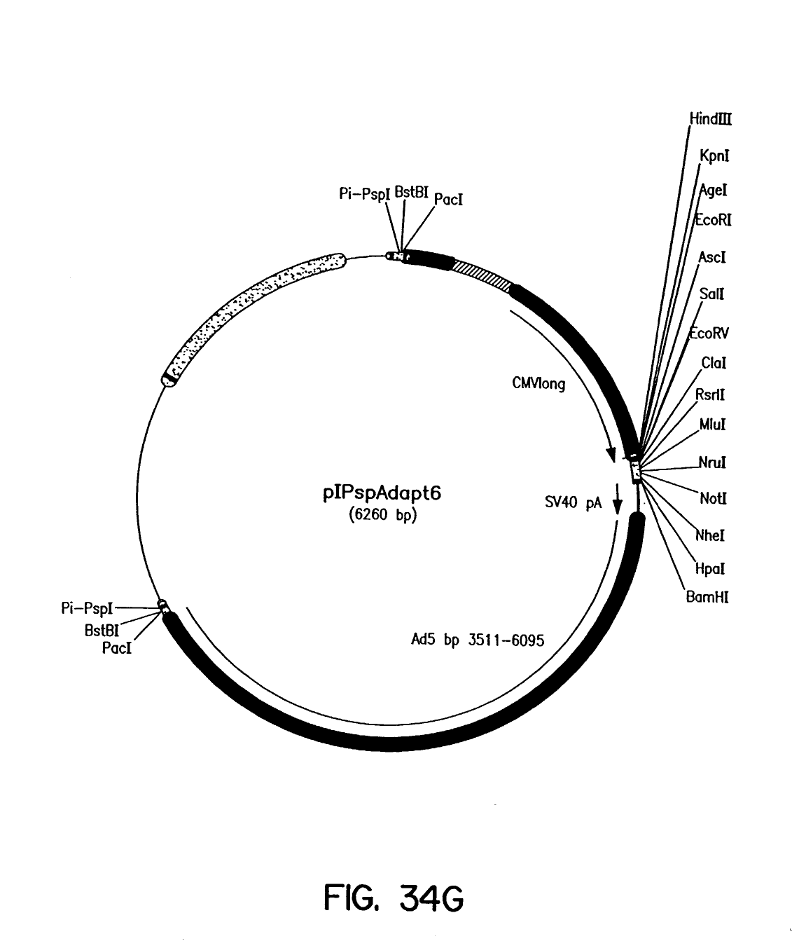

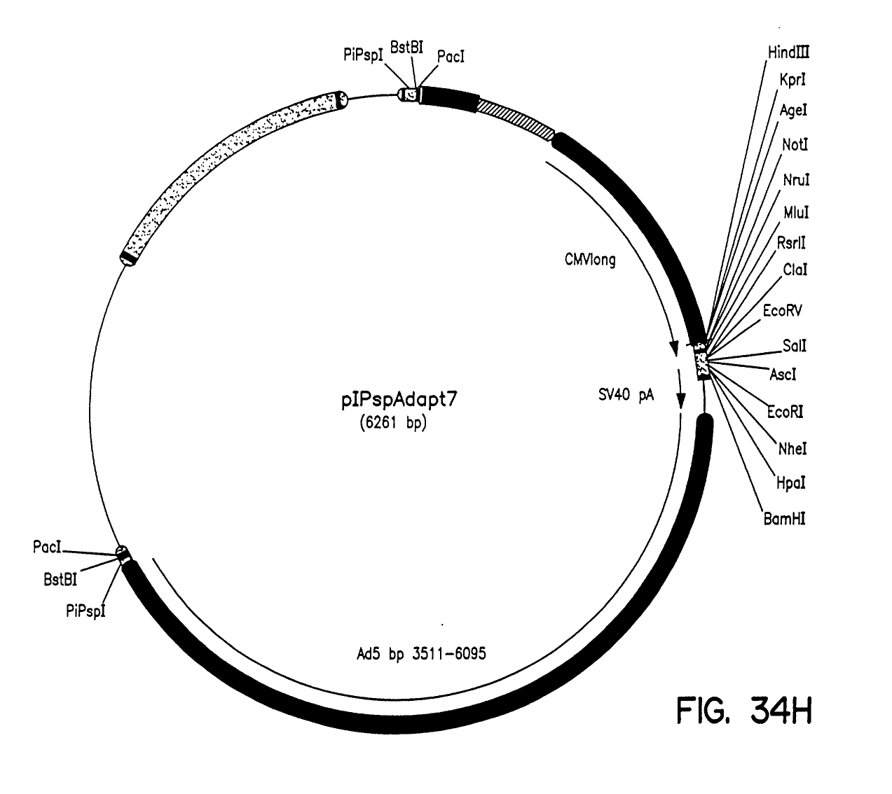

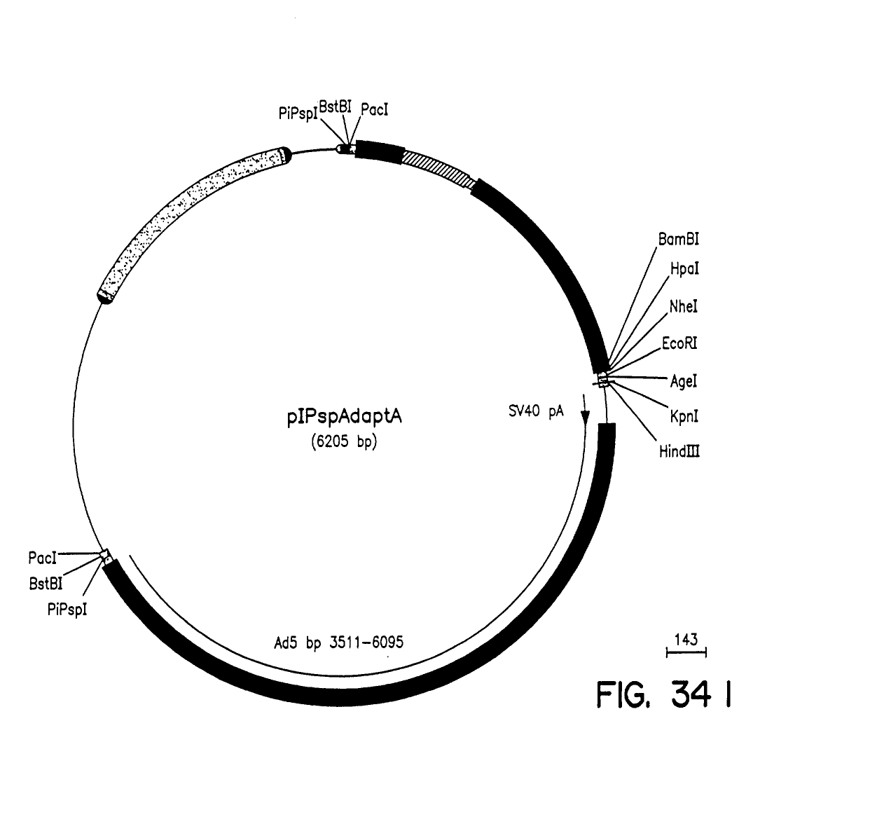

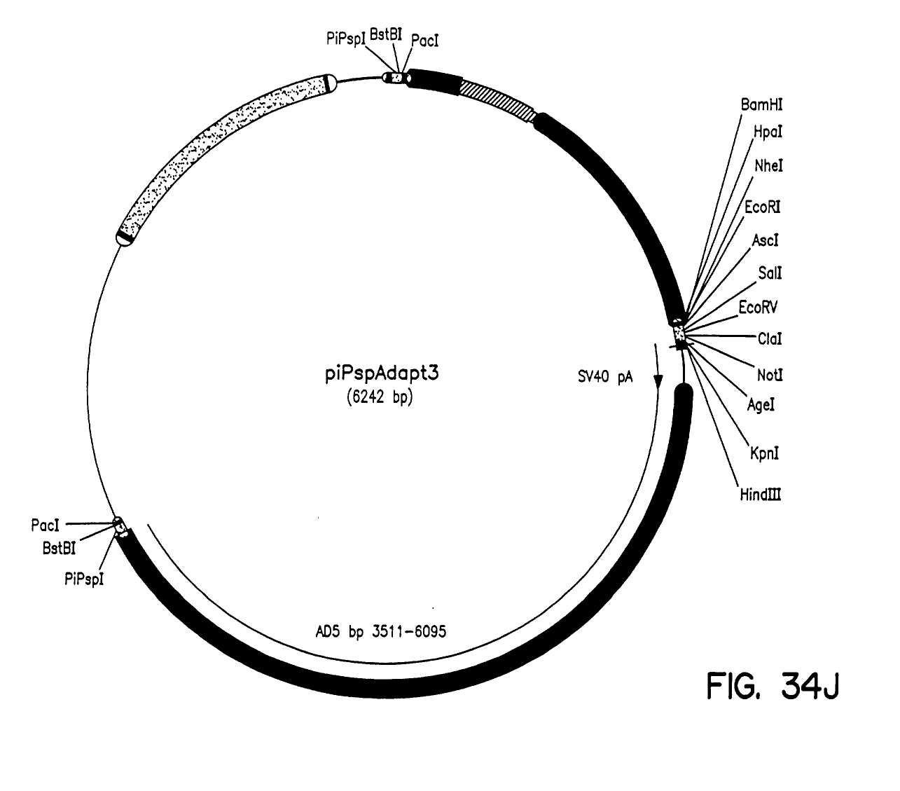

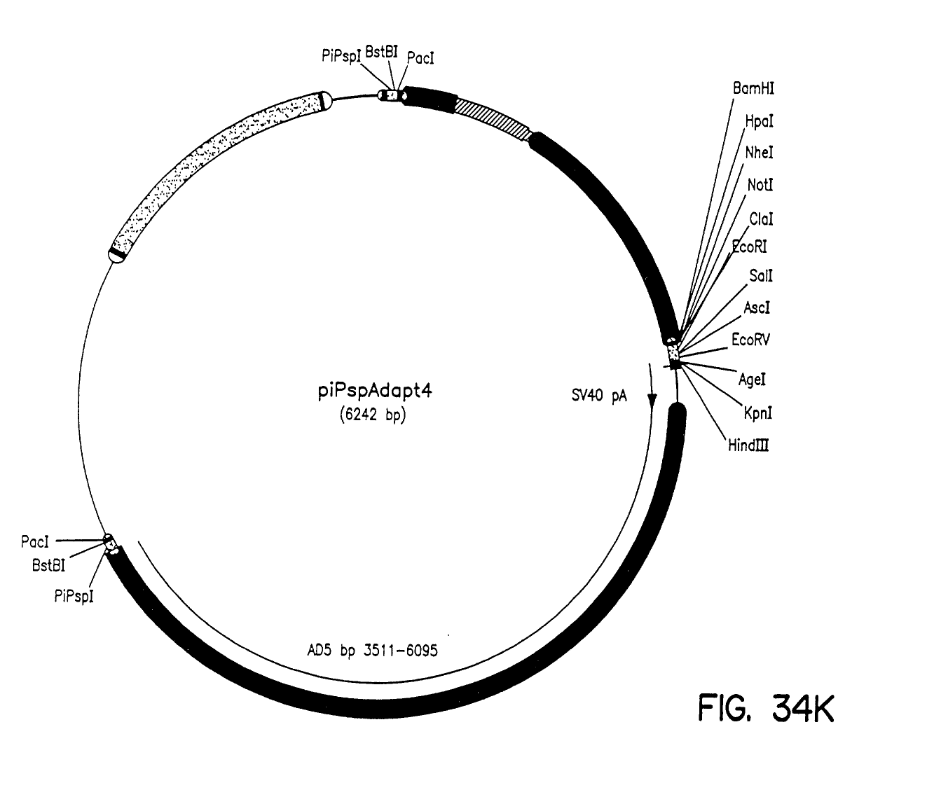

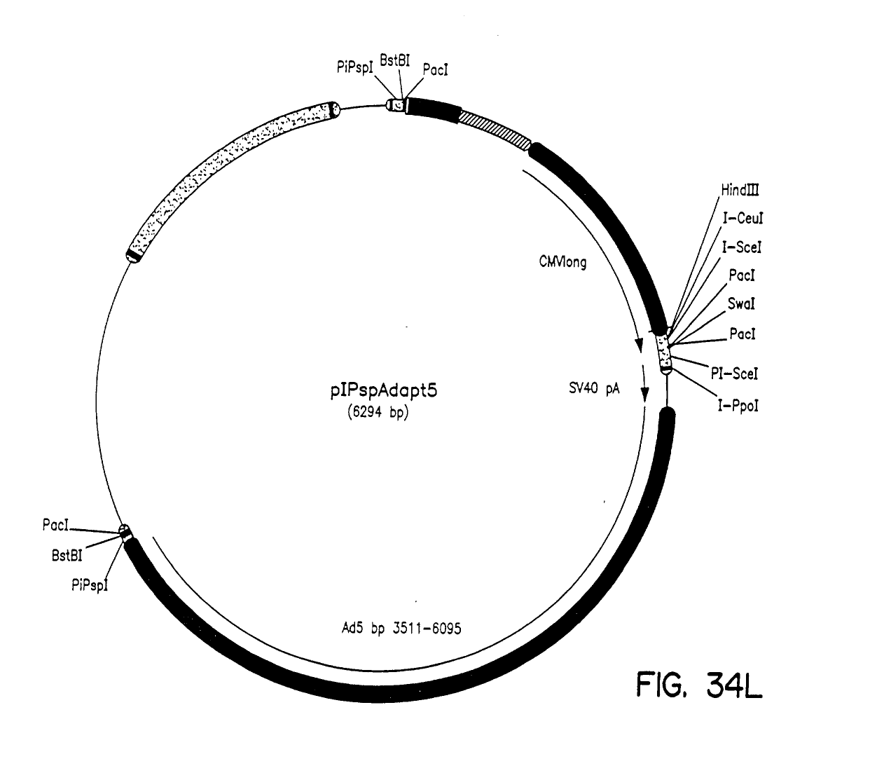

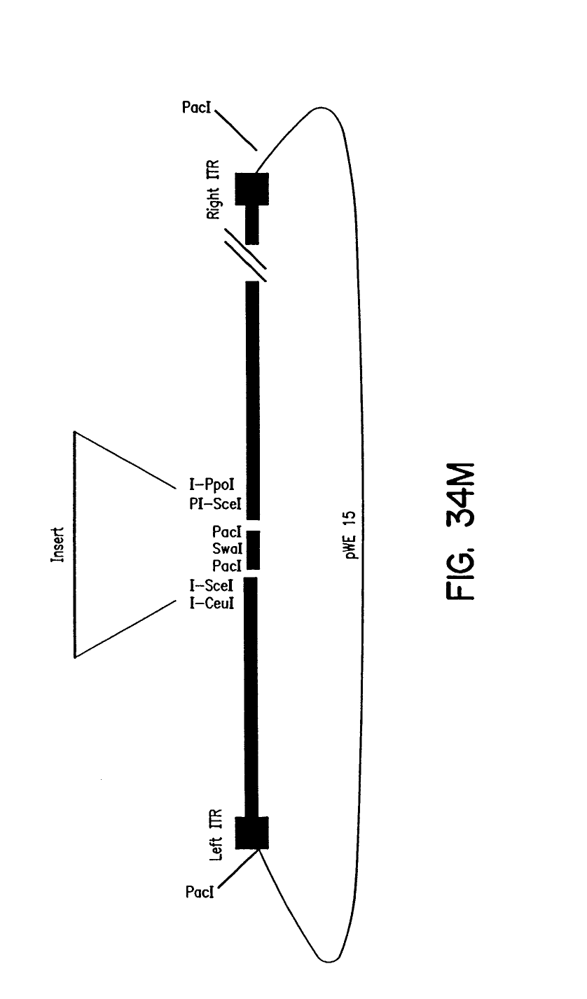

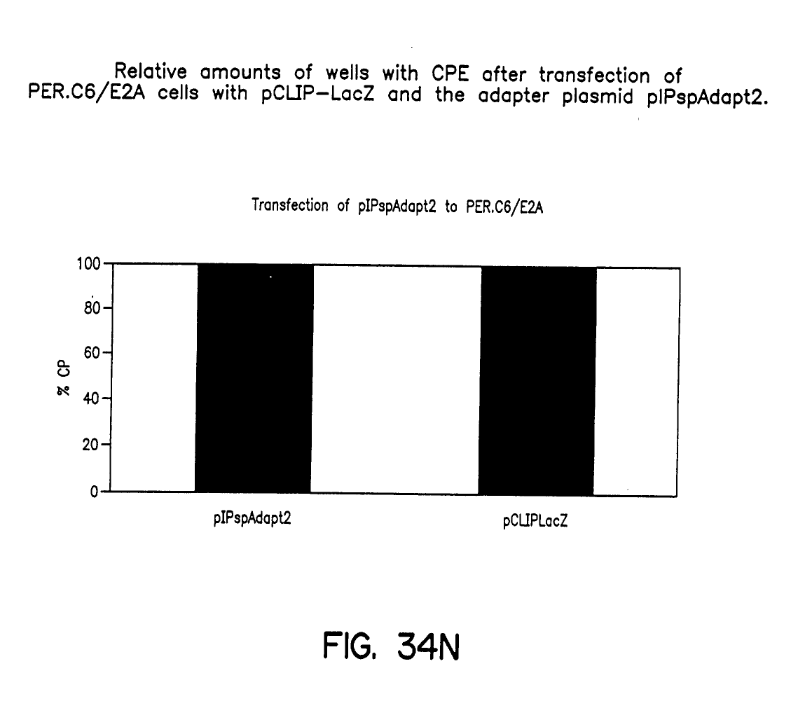

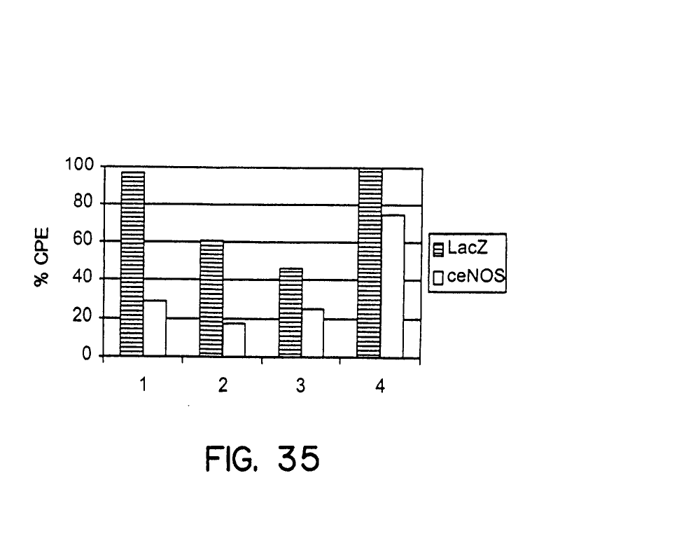

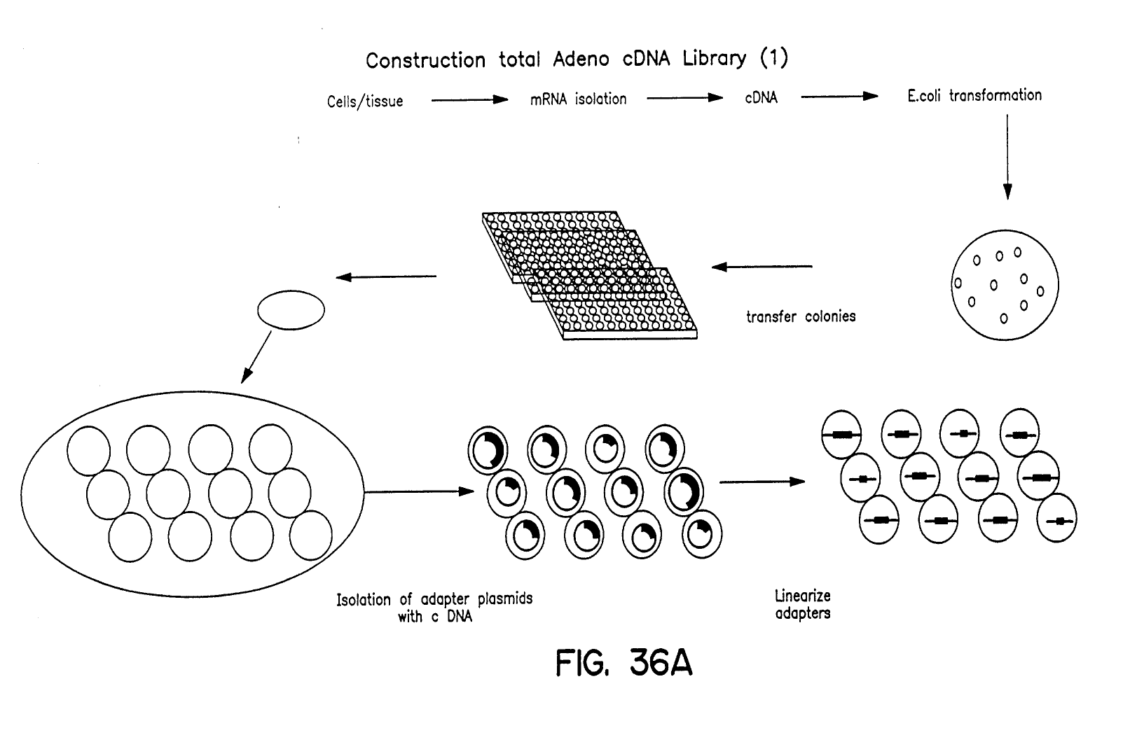

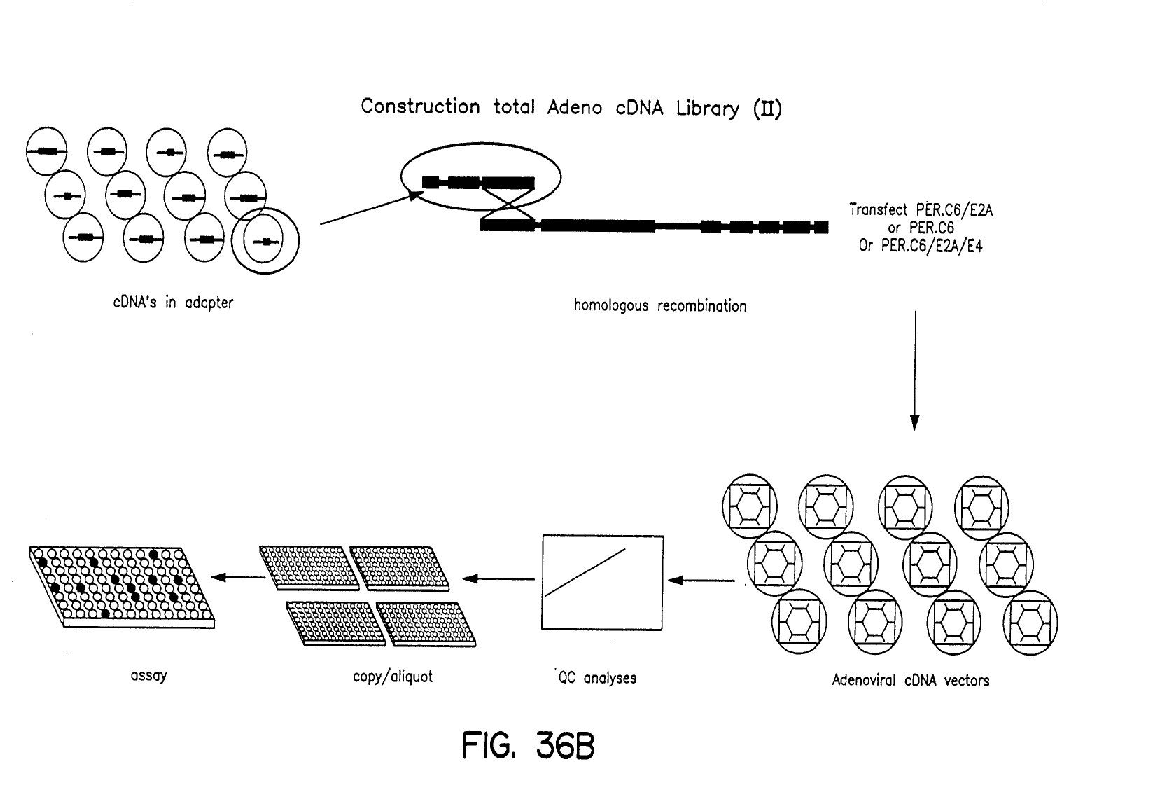

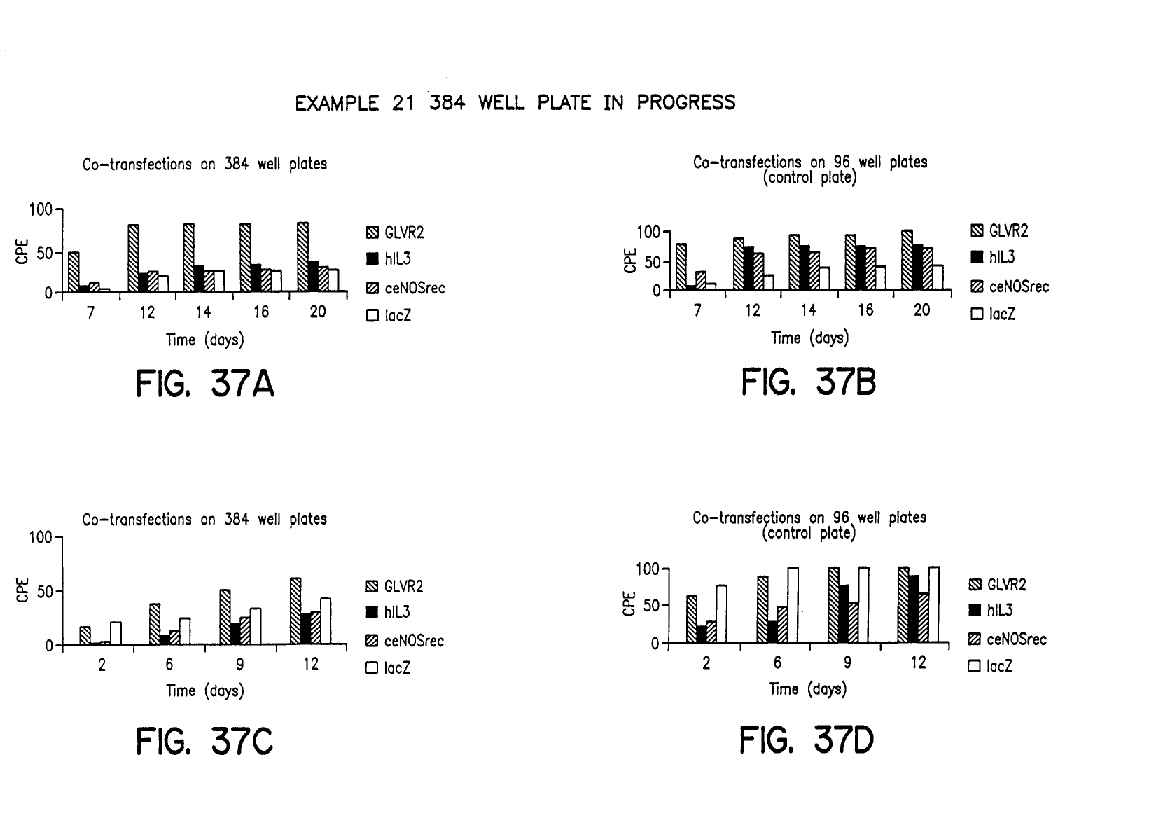

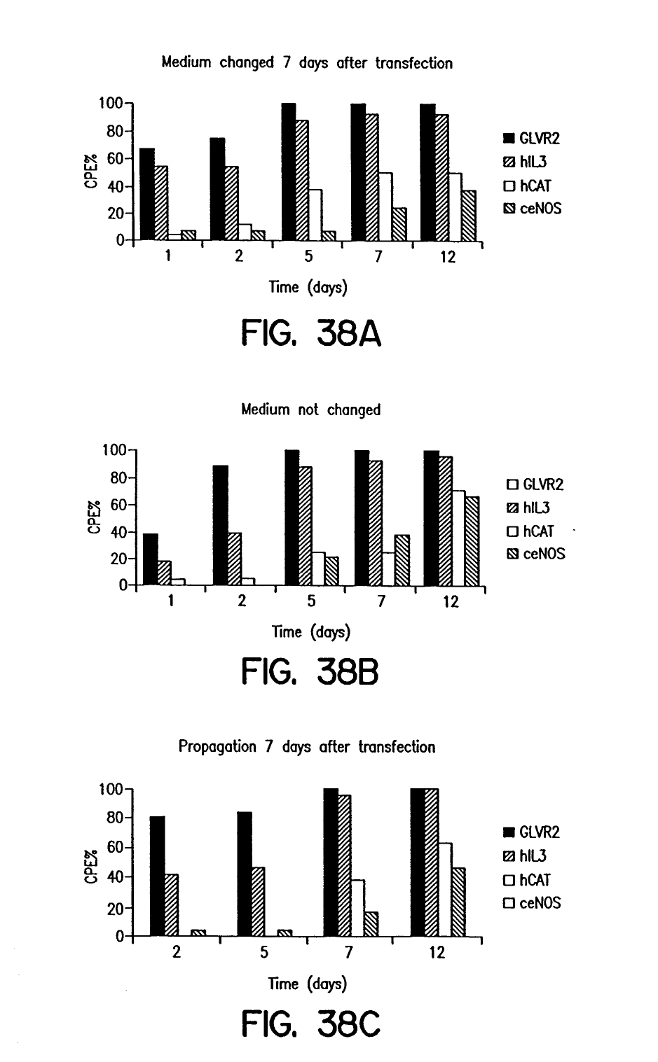

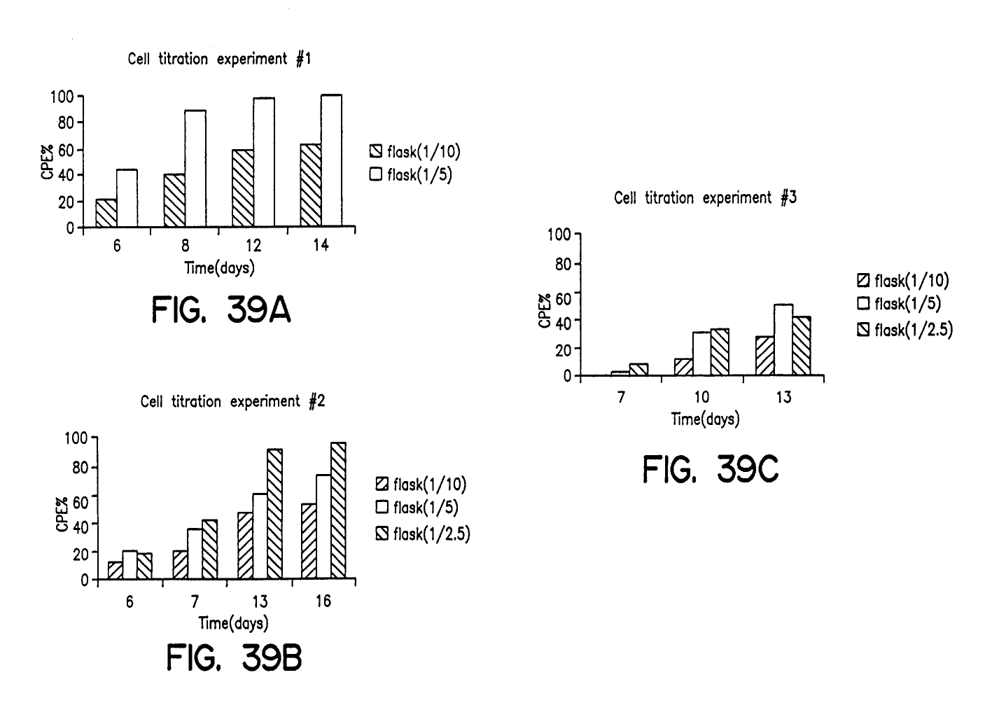

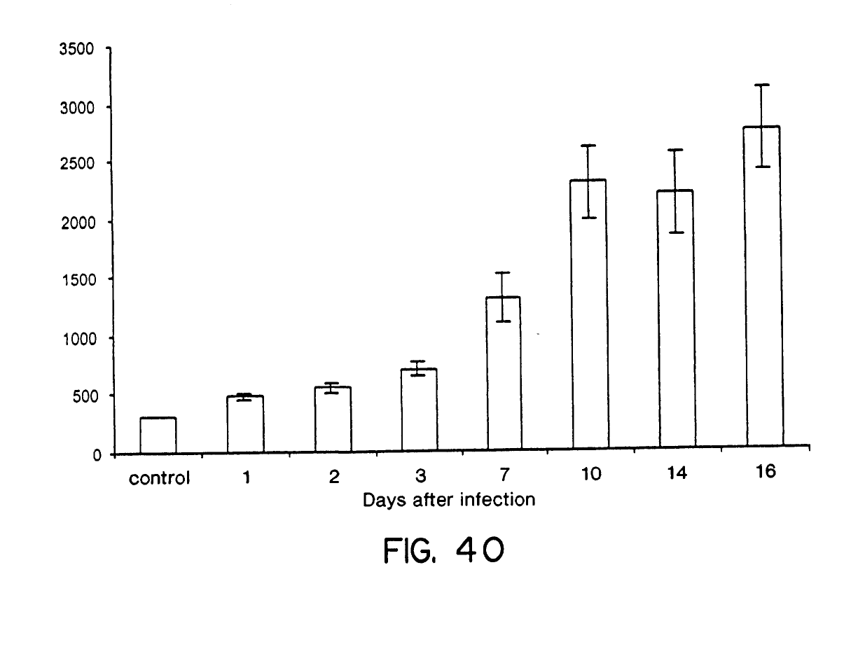

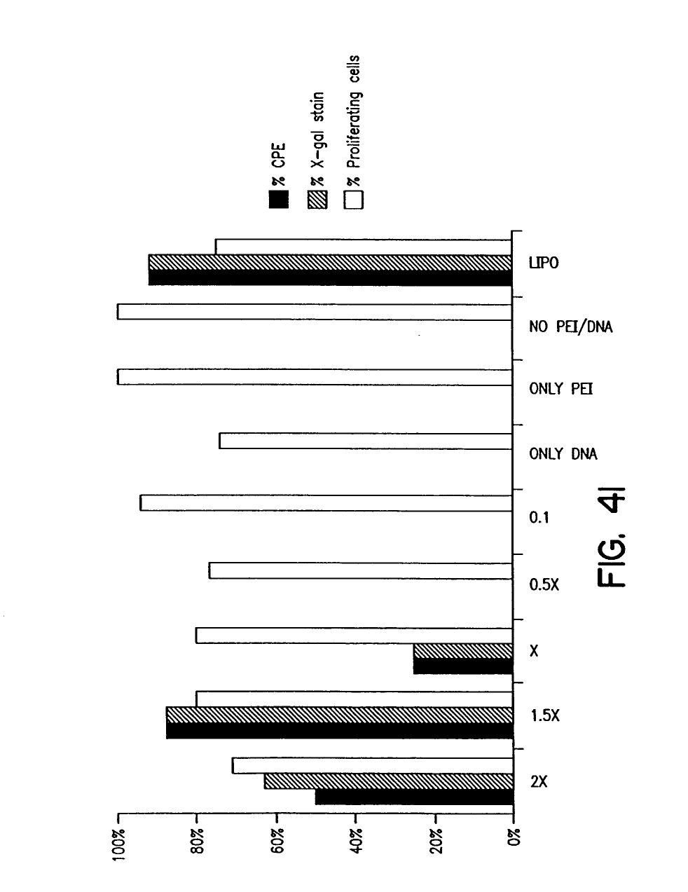

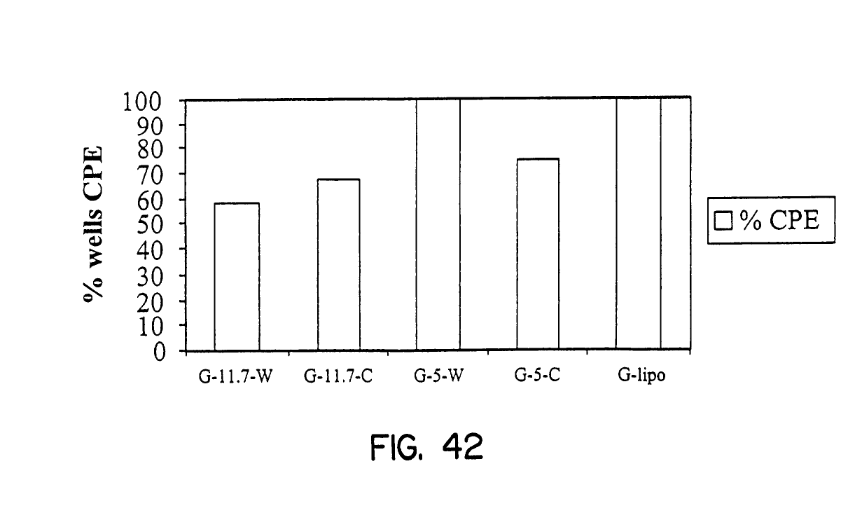

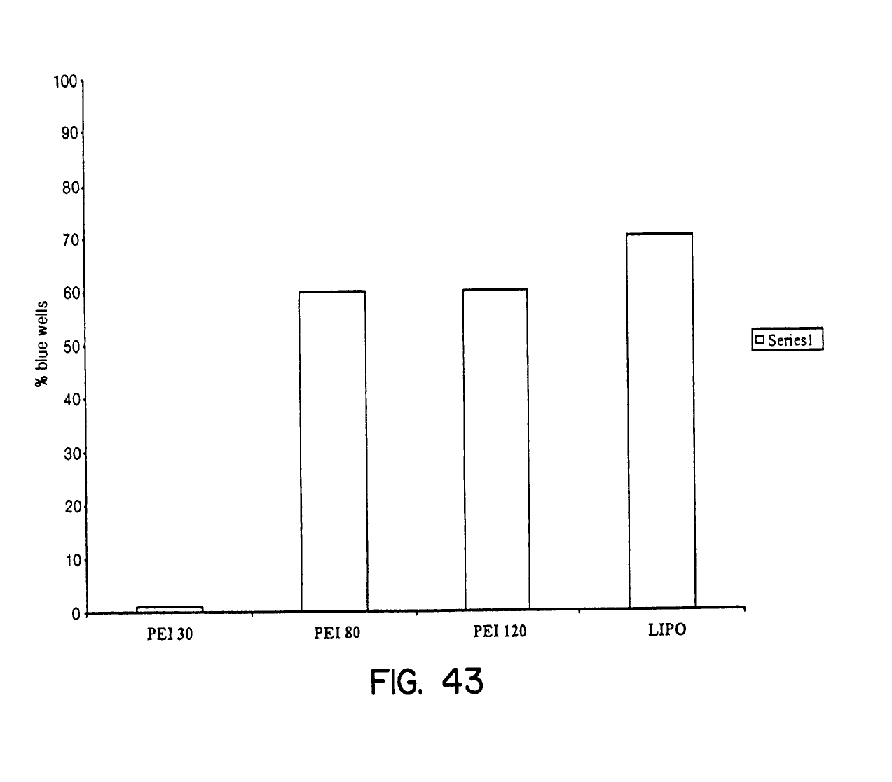

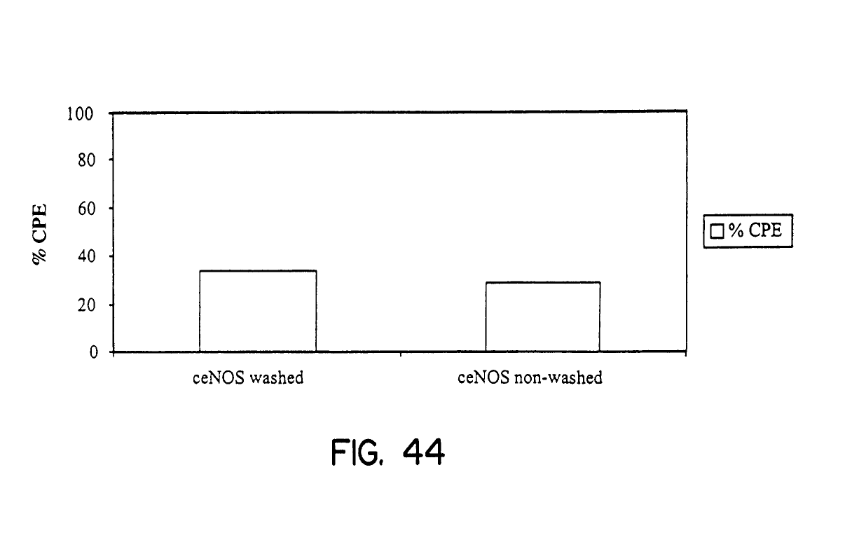

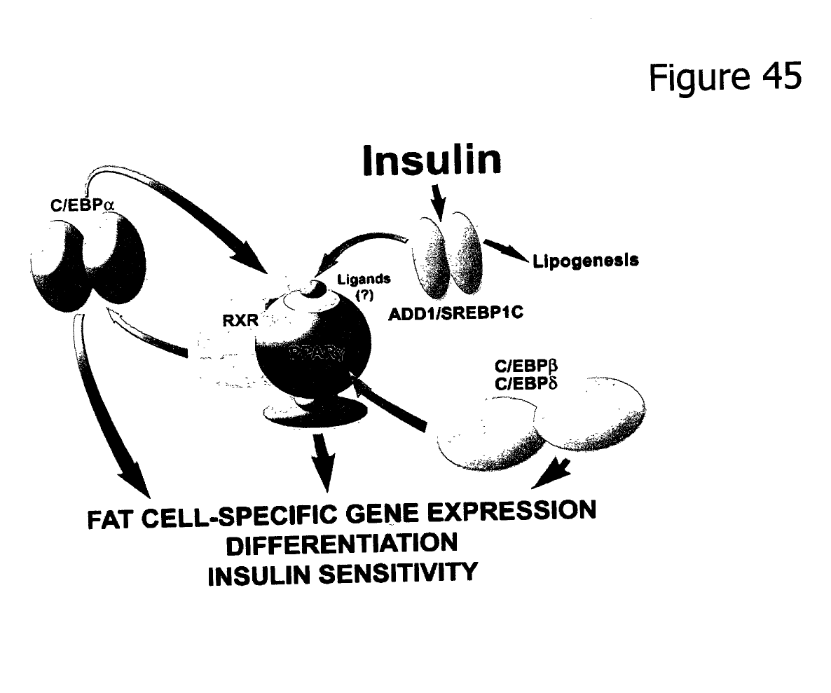

The vector into which the sample nucleic acids are preferably introduced contains, in an operable configuration, an ITR, at least one cloning site or preferably a multiple cloning site for insertion of a library of sample nucleic acids, and transcriptional regulatory elements for delivery and expression of the sample nucleic acids in a host. It generally does not contain E1 region sequences, E2B region sequences (other than those required for late gene expression), E2A region sequences, E3 region sequences, or E4 region sequences. The E1-deleted delivery vector or adapter plasmid is digested with the appropriate restriction enzymes, either simultaneously or sequentially, to produce the appropriate ends for directional cloning of the sample nucleic acid whether it be synthetic double-stranded oligonucleotides, genomic DNA, cDNA, ESTs, or a previously-cloned DNA. Restriction enzyme digestion is routinely performed using commercially available reagents according to the manufacturer's recommendations and varies according to the restriction enzymes chosen. A distinct set or pool of sample nucleic acids is inserted into E1-deleted adapter plasmids to produce a corresponding set or library of plasmids for the production of adenoviral vectors. An example of an adapter plasmid is pMLPI.TK, which is made up of adenoviral nucleotides 1-458 followed by the adenoviral major late promoter, functionally linked to the herpes simplex virus thymidine kinase gene, and followed by adenoviral nucleotides 3511-6095. Other examples of adapter plasmids are pAd/L420-HSA (FIG. 21) and pAd/Clip (FIG. 22). pAd/L420-HSA contains adenoviral nucleotides 1-454, the L420 promoter linked to the murine HSA gene, a poly-A signal, and adenoviral nucleotides 3511-6095. pAd/CLIP is made from pAd/L420-HSA by replacement of the expression cassette (L420-HSA) with the CMV promoter, a multiple cloning site, an intron, and a poly-A signal.

Once digested, the vector and sample nucleic acids are purified by gel electrophoresis. The nucleic acids can be extracted from various gel matrices by, for example, agarose digestion, electroelution, melting, or high salt extraction. In combination with these methods or alternatively, the digested nucleic acids can be purified by chromatography (e.g., Qiagen or equivalent DNA binding resins) or phenol-chloroform extraction followed by ethanol precipitation. The optimal purification method depends on the size and type of the vector and sample nucleic acids. Both can be used without purification. Generally, the sample nucleic acids contain 5′-phosphate groups and the vector is treated with alkaline phosphatase to promote nucleic acid-vector ligation and prevent vector-vector ligation. If the sample nucleic acid is a synthetic oligonucleotide, 5′-phosphate groups are added by chemical or enzymatic means. For ligation, molar ratios of sample nucleic acids (insert) to vector DNA range from approximately 10:1 to 0.1:1. The ligation reaction is performed using T4 DNA ligase or any other enzyme that catalyzes double-stranded DNA ligation. Reaction times and temperature can vary from about 5 minutes to 18 hours, and from about 15° C. to room temperature, respectively.

The magnitude of expression can be modulated using promoters (CMV immediately early, promoter, SV40 promoter, or retrovirus LTRs) that differ in their transcriptional activity. Operatively linking the sample nucleic acid to a strong promoter such as the CMV immediate early promoter and optionally one or more enhancer element(s) results in higher expression allowing the use of a lower multiplicity of infection to alter the phenotype of a host. The option of using a lower multiplicity of infection increases the number of times a library can be used in situ, in vitro, and in vivo. Moreover, the lower the multiplicity of infection and dosages used in screening programs, assays, and animal models decreases the chance of eliciting toxic effects in the transduced host, which increases the reliability of the subject of this invention. Another way to reduce possible toxic effects relating to expression of the library is to use a regulatable promoter, particularly one which is repressed during virus production but can be turned on or is active during functional screenings with the adenoviral library, to provide temporal and/or cell type specific control throughout the screening assay. In this way, sample nucleic acids that are members of the library and are toxic, inhibitory, or in any other way interfere with adenoviral replication and production, can still be produced as an adenoviral vector (see WO 97/20943). Examples of this type of promoter are the AP1-dependent promoters which are repressed by adenoviral E1 gene products, resulting in shut off of sample nucleic acid expression during adenoviral library production (see van Dam, et al. (1990) Mol. Cell. Biol. 10(11):5857-64). Such a promoter can be made using combinatorial techniques or natural or adapted forms of promoters can be included. Examples of AP1-dependent promoters are promoters from the collagenase, c-myc, monocyte chemoattractant protein (JE or mcp-1/JE), and stromelysin genes (Hagmeyer, et al. (1993) EMBO J. 12(9):3559-72; Offringa, et al. (1990) Cell 62(23):527-38; Offringa, et al. (1988) Nucleic Acids Res. 16(23): 10973-84; van Dam, et al. (1989) Oncogene 4(10): 1207-12). Alternatively, other more specific but stronger promoters can be used especially when complex in vitro screenings or in vivo models are employed and tissue-regulated expression is desired. Any promoter/enhancer system functional in the chosen host can be used. Examples of tissue-regulated promoters include promoters with specific activity or enhanced activity in the liver, such as the albumin promoter (Tronche, et al. (1990) Mol. Biol. Med. 7(2): 173-85). Another approach to enhanced expression is to increase the half-life of the mRNA transcribed from the sample nucleic acids by including stabilizing sequences in the 3′ untranslated region. A second nucleic acid construct, a helper plasmid having sequences homologous to sequences in the E1-deleted adapter plasmids, which carries all necessary adenoviral genes necessary for replication and packaging, also is prepared. Preferably, the helper plasmid has no complementing sequences that are expressed by the packaging cells that would lead to production of RCA. In addition, preferably the helper plasmids, adapter plasmid, and packaging cell have no sequence overlap that would lead to homologous recombination and RCA formation. The region of sequence overlap shared between the adapter plasmid and the helper plasmid allows homologous recombination and the formation of an E1-deleted, replication-defective recombinant adenoviral genome. Generally, the region of overlap encompasses E2B region sequences that are required for late gene expression. The amount of overlap that provides for the best efficiency without appreciably decreasing the size of the library insert can be determined empirically. The sequence overlap is generally 10 bp to 5000 bp, more preferably 2000 to 3000 bp.

The size of the sample nucleic acids or DNA inserts in a desired adenoviral library can vary from several hundred base pairs (e.g., sequences encoding neuropeptides) to more than 30 kb (e.g., titin). The cloning capacity of the adenoviral vectors produced using adapter plasmids can be increased by deletion of additional adenoviral gene(s) that are then easily complemented by a derivative of an E1-complementing cell line. As an example, candidate genes for deletion include E2, E3, and/or E4. For example, regions essential for adenoviral replication and packaging are deleted from the adapter and helper plasmids and expressed, for example, by the complementing cell line. For E3 deletions, genes in this region do not need to be complemented in the packaging cell for in vitro models; in in vivo models, the impact upon immunogenicity of the recombinant virus can be assessed on an ad hoc basis.

The set or library of specific adapter plasmids or pool(s) of adapter plasmids is converted to a set or library of adenoviral vectors. The adapter plasmids containing the sample nucleic acids are linearized and transfected into an E1-complementing cell line. The adapter plasmids are preferably seeded in microtiter tissue culture plates with 96, 384, 1,536 or more wells per plate, together with helper plasmids having sequences homologous to sequences in the adapter plasmid and containing all adenoviral genes necessary for replication and packaging. Recombination occurs between the homologous sequences shared by adapter and helper plasmids to generate an E1-deleted, replication-defective adenoviral genome that is replicated and packaged, preferably, in an E1-complementing cell line. If more than one helper plasmid is used, recombination between homologous regions shared among the helper plasmids and recombination between the helper plasmids and adapter plasmid results in the formation of a replication-defective recombinant adenoviral genome. The regions of sequence overlap between the adapter and helper plasmids are at least about a few hundred nucleotides or greater. Recombination efficiency will increase by increasing the size of the overlap.

The E1-functions provided by the trans complementing packaging cell permit the replication and packaging of the E1-deleted recombinant adenoviral genome. The adapter and/or helper plasmids preferably have no sequence overlap amongst themselves or with the complementing sequences in the packaging cells that can lead to the formation of RCA. Preferably, at least one of the ITRs on the adapter and helper plasmids is flanked by a restriction enzyme recognition site not present in the adenoviral sequences or expression cassette so that the ITR is freed from vector sequences by digestion of the DNA with that restriction enzyme. In this way, initiation of replication occurs more efficiently. In order to increase the efficiency of recombinant adenoviral generation, higher throughput can be obtained by using microtiter tissue culture plates with 96, 384, or 1,536 wells per plate instead of using large tissue culture vials or flasks. E1-complementing cell lines are grown in microtiter plates and cotransfected with the libraries of adapter plasmids and a helper plasmid(s). Automation of the method using, for example, robotics can further increase the number of adenoviral vectors that can be produced (Hawkins, et al. (1997) Science 276(5320):1887-9; Houston, (1997) Methods Find. Exp. Clin. Pharmacol. 19 Suppl. A:43-5).

As an alternative to the adapter plasmids, the sample nucleic acids can be ligated to “minimal” adenoviral vector plasmids. The so-called “minimal” adenoviral vectors, according to the present invention, retain at least a portion of the viral genome that is required for encapsidation of the genome into virus particles (the encapsidation signal). The minimal vectors also retain at least one copy of at least a functional part or a derivative of the ITR, that is DNA sequences derived from the termini of the linear adenoviral genome that are required for replication. The minimal vectors preferably are used for the generation and production of helper- and RCA-free stocks of recombinant adenoviral vectors and can accommodate up to 38 kb of foreign DNA. The helper functions of the minimal adenoviral vectors are preferably provided in trans by encapsidation-defective, replication-competent DNA molecules that contain all the viral genes encoding the required gene products, with the exception of those genes that are present in the complementing cell or genes that reside in the vector genome.

Packaging of the “minimal” adenoviral vector is achieved by cotransfection of an E1-complementing cell line with a helper virus or, alternatively, with a packaging deficient replicating helper system. Preferably, the packaging deficient replicating helper is amplified following transfection and expresses the sequences required for replication and packaging of the minimal adenoviral vectors that are not expressed by the packaging cell line. The packaging deficient replicating helper is not packaged into adenoviral particles because its size exceeds the capacity of the adenoviral virion and/or because it lacks a functional encapsidation signal. The packaging deficient replicating helper, the minimal adenoviral vector, and the complementing cell line, preferably, have no region of sequence overlap that permits RCA formation.

The replicating, packaging deficient helper molecule always contains all adenoviral coding sequences that produce proteins necessary for replication and packaging, with or without the coding sequences provided by the packaging cell line. Replication of the helper molecule itself may or may not be mediated by adenoviral proteins and ITRs. Helper molecules that replicate through the activity of adenoviral proteins (for example, E2-gene products acting together with cellular proteins) contain at least one ITR derived from adenovirus. The E2-gene products can be expressed by an E1-dependent or an E1-independent promoter. Where only one ITR is derived from an adenovirus, the helper molecule also preferably contains a sequence that anneals in an intramolecular fashion to form a hairpin-like structure.

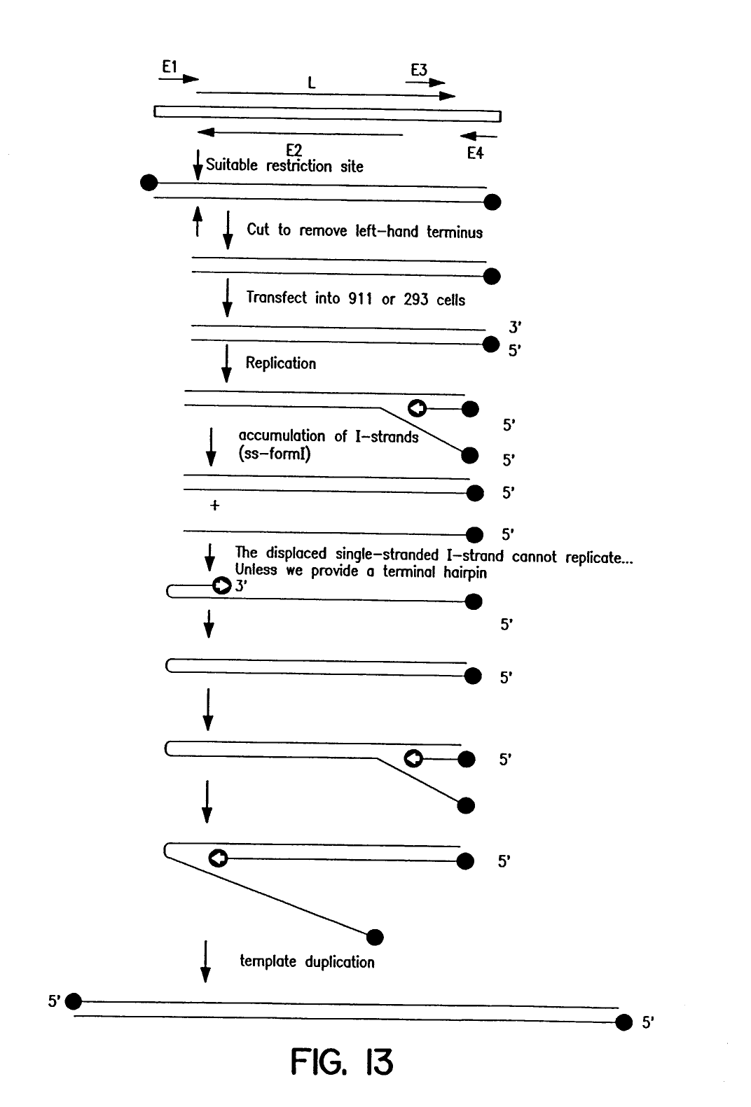

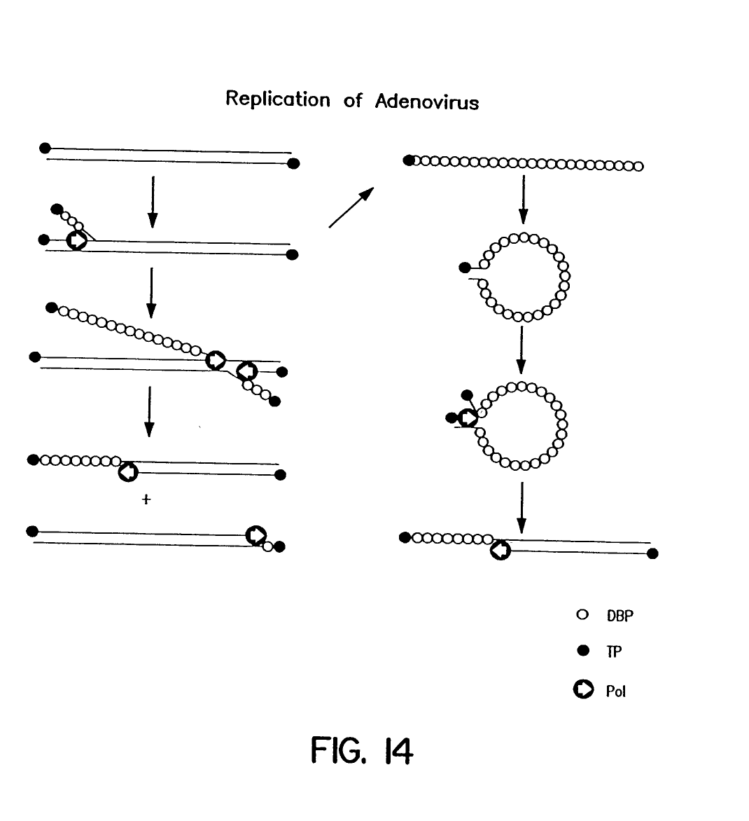

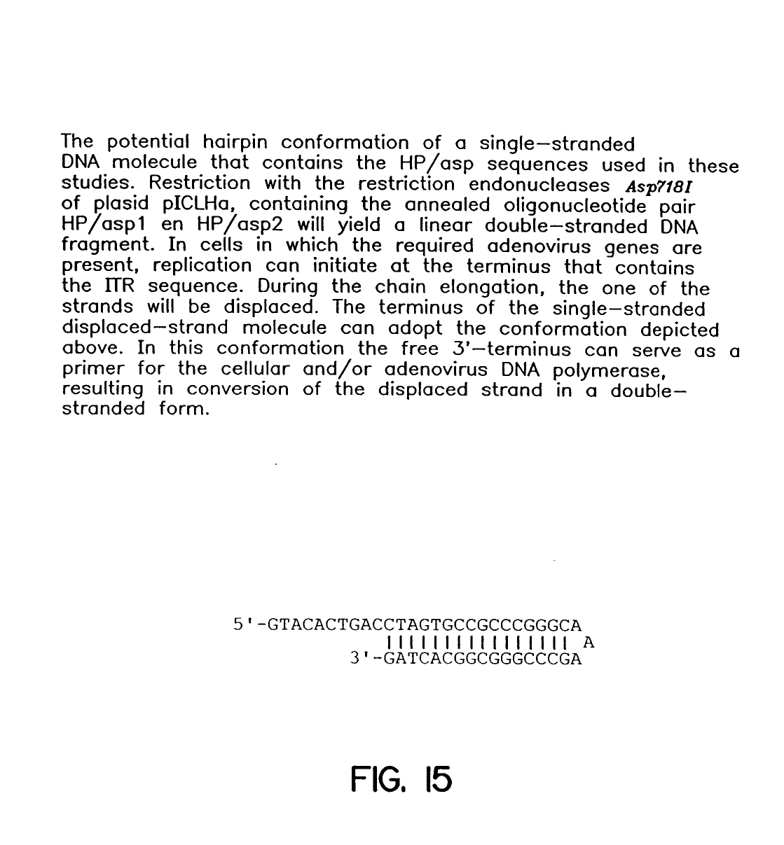

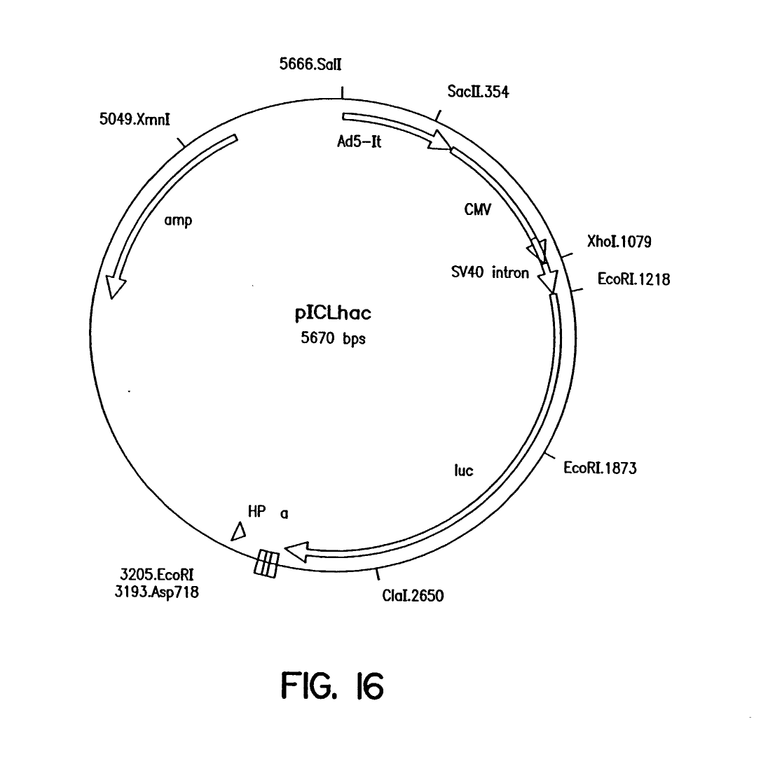

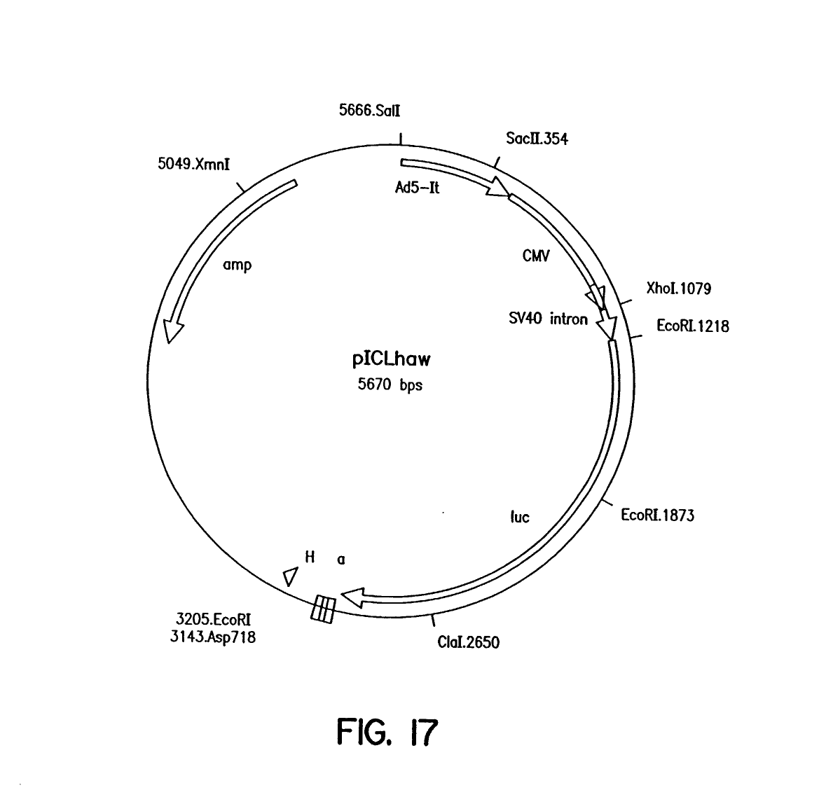

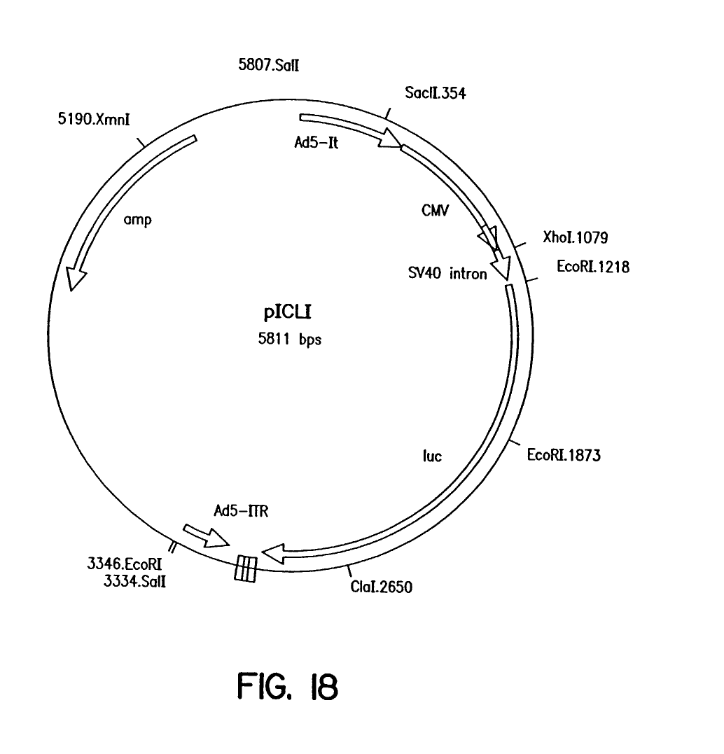

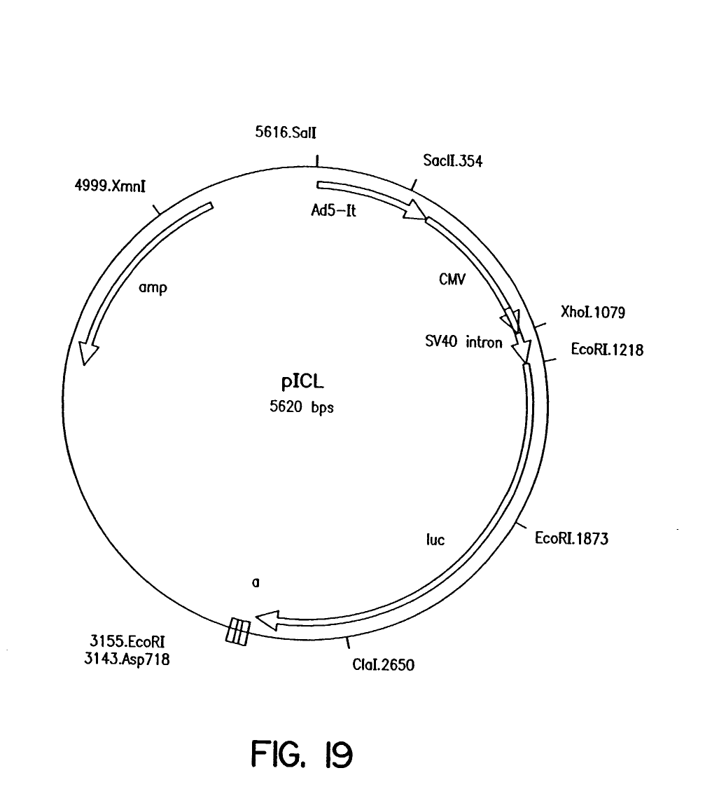

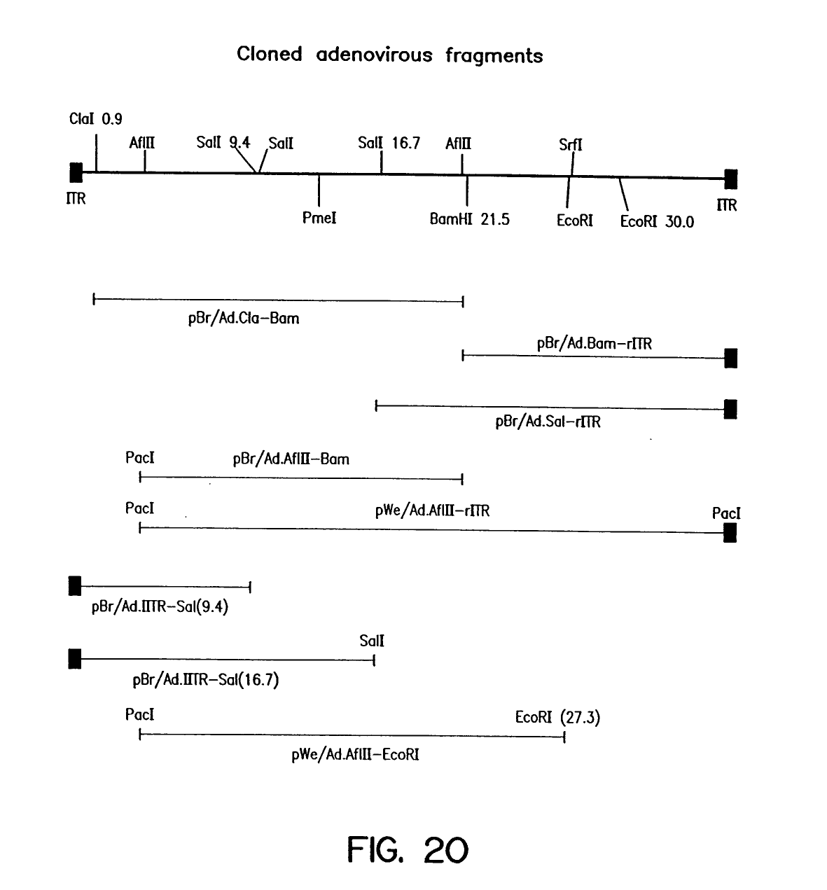

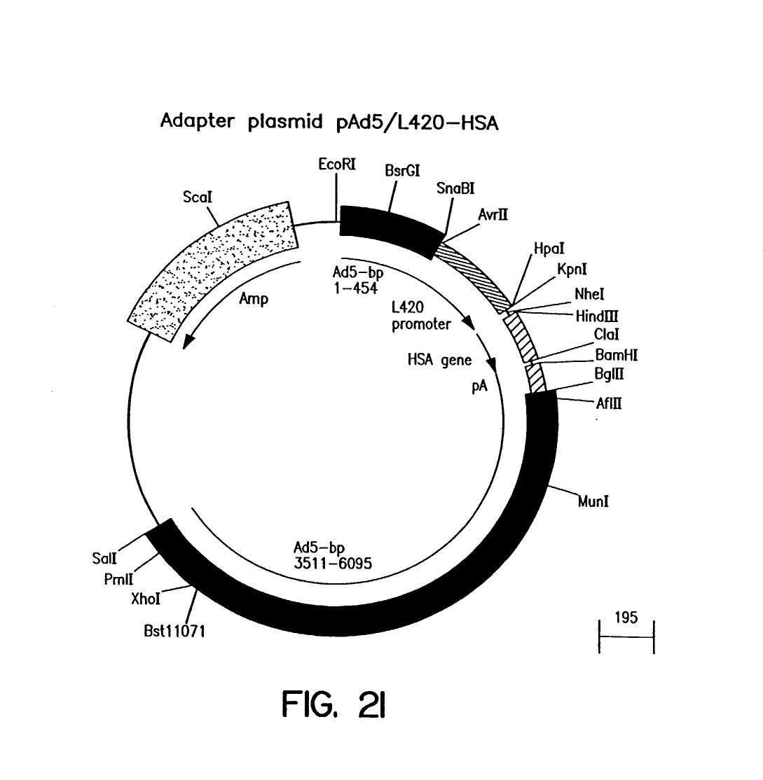

Following E2-gene product expression, the adenoviral DNA polymerase recognizes the ITR on the helper molecule and produces a single-stranded copy. Then, the 3′-terminus intramolecularly anneals, forming a hairpin-like structure that serves as a primer for reverse strand synthesis. The product of reverse strand synthesis is a double-strand hairpin with an ITR at one end. This ITR is recognized by adenoviral DNA polymerase that produces a double-stranded molecule with an ITR at both termini (see e.g., FIG. 13) and becomes twice as long as the transfected molecule (in our example it duplicates from 35 Kb to 70 Kb). Duplication of the helper DNA enhances the production of sufficient levels of adenoviral proteins. Preferably, the size of the duplicated molecule exceeds the packaging capacity of the adenoviral virion and is, therefore, not packaged into adenoviral particles. The absence of a functional encapsidation signal in the helper molecule further ensures that the helper molecule is packaging deficient. The produced adenoviral proteins mediate replication and packaging of the cotransfected or co-infected minimal vectors.

When the replication of the helper molecule is independent of adenoviral E2-proteins, the helper construct preferably contains an origin of replication derived from SV40. Transfection of this DNA, together with the minimal adenoviral vector in an E1-containing packaging cell line that also inducibly expresses the SV40 Large T protein or as much SV40 derived proteins as necessary for efficient replication, leads to replication of the helper construct and expression of adenoviral proteins. The adenoviral proteins then initiate replication and packaging of the co-transfected or co-infected minimal adenoviral vectors. Preferably, there are no regions of sequence overlap shared by the replication-deficient packaging constructs, the minimal adenoviral vectors, and the complementing cell lines that may lead to the generation of RCA.

It is to be understood that during propagation of the minimal adenoviral vectors, each amplification step on E1-complementing cells is preceded by transfection of any of the described helper molecules since minimal vectors by themselves cannot replicate on E1-complementing cells. Alternatively, a cell line that contains all the adenoviral genes necessary for replication and packaging, which are stably integrated in the genome and can be excised and replicated conditionally, can be used (Valerio and Einerhand, International patent Appl'n PCT/NL9800061).

Transfection of nucleic acid into cells is required for packaging of recombinant vectors into virus particles and can be mediated by a variety of chemicals including liposomes, DEAE-dextran, polybrene, and phosphazenes or phosphazene derivatives (WO 97/07226). Liposomes are available from a variety of commercial suppliers and include DOTAP® (Boehringer-Mannheim), Tfx®-50, Transfectam®, ProFectione ® (Promega, Madison, Wis.), and LipofectAmine®, Lipofectin®, LipofectAce® (GibcoBRL, Gaithersburg, Md.). In solution, the lipids form vesicles that associate with the nucleic acid and facilitate its transfer into cells by fusion of the vesicles with cell membranes or by endocytosis. Other transfection methods include electroporation, calcium phosphate coprecipitation, and microinjection. If transfection conditions for a given cell line have not been established or are unknown, they can be determined empirically (Maniatis, et al. Molecular Cloning, pages 16.30-16.55).

The yield of recombinant adenoviral virus vectors can be increased by denaturing the double stranded plasmid DNA before transfection into an E1 complementing cell line. Denaturing can occur by heating double-stranded DNA at, for example, 95-100° C., followed by rapid cooling using various ratios of the adapter and helper plasmids that have overlapping sequences. Also, a PER.C6 derivative that stably or transiently expresses E2A (DNA binding protein) and/or E2B gene (pTP-Pol) could be used to increase the adenoviral production per well by increasing the replication rate per cell. Alternatively, cotransfection of recombinase protein(s), recombinase DNA expression construct(s), i.e. recombinase from Kluyveromyces waltii (Ringrose, et al. (1997) Eur. J. Biochem. 248(3):903-12), or any other gene or genes encoding factors that can increase homologous recombination efficiency can be used. The inclusion of 0.1-100 mM sodium butyrate during transfection and/or replication of the packaging cells can increase viral production. These improvements will result in improved viral yields per microtiter well. Therefore, the number and type of assays that can be done with one library will increase and may overcome variability between the various genes or sample nucleic acids in a library.

The cell lines used for the production of adenoviral vectors that express E1 region products includes, for example, 293 cells, PER.C6 (ECACC 96022940), or 911 cells. Each of these cell lines has been transfected with nucleic acids that encode for the adenoviral E1 region. These cells stably express E1 region gene products and have been shown to package El-deleted recombinant adenoviral vectors. Yields of recombinant adenovirus obtained on PER.C6 cells are higher than obtained on 293 cells.

Each of these cell lines provides the basis for introduction of E2B, E2A, or E4 constructs (e.g., tsl25E2A and/or hrE2A) that permit the propagation of adenoviral vectors that have mutations, deletions, or insertions in the corresponding genes. These cells can be modified to express additional adenoviral gene products by the introduction of recombinant nucleic acids that stably express the appropriate adenoviral genes or recombinant nucleic acids and that transiently express the appropriate gene(s), for example, the packaging deficient replicating helper molecules or the helper plasmids.

All (or nearly all) trans complementing cells grown in microtiter plate wells (96, 384, or more than 1,536 wells) produce recombinant adenovirus following transfection with either the adapter plasmid or the minimal adenoviral plasmid library and the appropriate helper molecule(s). A large number of adenoviral gene transfer vectors or a library, each expressing a unique gene, can thus be conveniently produced on a scale that allows analysis of the biological activity of the particular gene products both in vitro and in vivo. Due to the wide tissue tropism of adenoviral vectors, a large number of cell and tissue types are transducible with an adenoviral library.

In one example, growth medium of the cell culture contains sodium butyrate in an amount sufficient to enhance production of the recombinant adenoviral vector library.

Preferably, the plurality of cells further includes at least one of an adenoviral preterminal protein and a polymerase complementing sequence. Preferably, the plurality of cells further includes an adenoviral E2 complementing sequence. Preferably, the E2 complementing sequence is an E2A complementing sequence or an E2B complementing sequence. In one aspect, the plurality of cells further includes a recombinase protein, whereby the homologous recombination leading to replication-defective, recombinant adenovirus is enhanced. Preferably, the recombinase protein is a Kluyveromyces waltii recombinase. In another aspect, the plurality of cells further includes a nucleotide sequence coding for a recombinase protein. Preferably, the recombinase protein is Kluyveromyces waltii recombinase.

Libraries of genes or sample nucleic acids preferably are converted to RCA free adenoviral libraries and used in the present invention in combination with high throughput screening of compounds involving a number of in vitro assays, such as immunological assays including ELISAs, proliferation assays, drug resistance assays, enzyme activity assays, organ cultures, differentiation assays, and cytotoxicity assays. Adenoviral libraries can be tested on tissues, tissue sections, or tissue derived primary short-lived cell cultures including primary endothelial and smooth muscle cell cultures (Wijnberg, et al. (1997) Thromb. Haemost. 78(2):880-6), coronary artery bypass graft libraries (Vassalli, et al. (1997) Cardiovasc. Res. 35(3):459-69; Fuster and Chesebro, (1985) Adv. Prostaglandin Thromboxane Leukot. Res. 13:285-99), umbilical cord tissue including HUVEC (Gimbrone, (1976) Prog. Hemost. Thromb. 3:1-28; Striker, et al. (1980) Methods Cell. Biol. 21A:135-51), couplet hepatocytes (Graf, et al. (1984) Proc. Natl. Acad. Sci. USA 81(20):6516-20), and epidermal cultures (Fabre, (1991) Immunol Lett. 29(1-2):161-5; Phillips, (1991) Transplantation 51(5):937-41). Plant cell cultures, including suspension cultures, can also be used as host cells for the adenoviral libraries carrying any DNA sequence, including human derived DNA sequences and plant derived sequences. (de Vries, et al. (1994) Biochem. Soc. Symp. 60:43-50; Fukada, et al. (1994) Int. J. Devel. Biol. 38(2):287-99; Jones, (1983) Biochem. Soc. Symp. 48:221-32; Kieran, et al. (1997) J. Biotechnol. 59(1-2):39-52; Stanley, (1993) Curr. Opin. Genet. Dev. 3(1):91-6; Taticek, et al. (1994) Curr. Opin. Biotechnol. 5(2):165-74.

In addition, in vitro assays can be complemented by using an electronic version of the sequence database on which the adenoviral library is built. This allows, for example, protein motif searching whereby new members of a family can be linked to known members of the same family with known functions. The use of Hidden Markow Models (HMMs) (Eddy, (1996) Proc. Natl. Acad. Sci. USA 94(4): 1414-9) allows the establishment of novel families by identifying essential features of a family and building a model of what the members should look like. This can be combined with structural data by using the threading approach, which uses a known structure as the thread and tries to find a putative structure without having determined the actual structure of the novel protein (Rastan and Beeley, (1997) Curr. Opin. Genet. Dev. 7(6):777-83). The functional data, which is obtained using adenoviral libraries made in accordance with the methods disclosed in this application, is the foundation of the endeavor to find novel genes with expected or desired functions and will be the core of functional genomics. Finally, once the number of adenoviral vectors has reached a level at which animal experiments can be performed, another addition to the method is to produce the- selection of candidate adenoviral vectors carrying the candidate genes. Then, the clones can be purified by, for example, using adenovirus tagged in the Hi loop of the knob domain of the fiber. Alternatively, large scale HPLC analysis can be used in a semipreparative fashion to yield partially purified adenoviral samples for in vivo or in vitro experiments when more purified adenoviral preparations are desired. Therefore, the described method and reagents allow rapid transfer of a collection of genes in in vivo studies of a limited number of animals, which otherwise would be unfeasible. The automation of the steps of the procedure using robotics will further enhance the number of genes and sample nucleic acids that can be functionated.

Aspects of the present invention include methods of assay and compositions used therein for the identification of compounds useful for the treatment of disease states that involve the processes of adipogenesis, i.e., the cellular differentiation into adipocytes, and the formation of lipid vacuoles in cells. Exemplary disease states are obesity, Type II diabetes, hyperglycemia, impaired glucose tolerance, metabolic syndrome, syndrome X, dyslipidemia, liposarcoma and insulin resistance.

The methods and compositions of the present invention are based on the identification of the polypeptides and polynucleotides discovered by the adenoviral library screening methods described hereinabove. By using these polypeptides and polynucleotides as targets in screening assays, such as high throughput screens, small molecule compounds can be identified as drug candidates for pharmaceutical development. As will be discussed in a subsequent section herein below, the present invention also relates pharmaceutical compositions and methods of treatment comprising these polypeptides and polynucleotides.

High Throughput Binding Screen for Compounds that Affect the Ability of the Identified Genes to Induce Lipid Droplet Formation

Screening assays for drug candidates are designed to identify compounds that bind or complex with the polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art.

All assays are common in that they call for contacting the drug candidate with a polypeptide or a polynucleotide that induces lipid droplet formation under conditions and for a time sufficient to allow these two components to interact.

In binding assays, the interaction is binding and the complex formed can be isolated or detected in the reaction mixture. In a particular embodiment, the polypeptide or polynucleotide that induces lipid droplet formation or the drug candidate is immobilized on a solid phase, e.g. on a microtiter plate, by covalent or non-covalent attachments. Non-covalent attachment generally is accomplished by coating the solid surface with a solution of the polypeptide or polynucleotide and drying. Alternatively, an immobilized antibody, e.g. a monoclonal antibody, specific for the polypeptide or polynucleotide to be immobilized can be used to anchor it to a solid surface. The assay is performed by adding the non-immobilized component, which may be labelled by a detectable label, to the immobilized component, e.g. the coated surface containing the anchored component. When the reaction is complete, the non-reacted components are removed, e.g. by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the candidate compound interacts with but does not bind to a polypeptide or polynucleotide that induces lipid droplet formation, its interaction with that molecule can be assayed by methods well known for detecting interactions. Such assays include traditional approaches, such as, cross-linking, co-immunoprecipitation, and co-purification through gradients or chromatographic columns.

To screen for antagonists and/or agonists of gene products identified herein, the assay mixture is incubated under conditions whereby, but for the presence of the candidate pharmacological agent, the identified gene product induces lipid droplet formation. The mixture components can be added in any order that provides for the requisite activity. Incubation may be performed at any temperature that facilitates optimal binding, typically between about 4° C. and 40° C., more commonly between about 15° C. and 40° C. Incubation periods are likewise selected for optimal binding but also minimized to facilitate rapid, high-throughput screening, and are typically between about 0.1 and 10 hours, preferably less than 5 hours, more preferably less than 2 hours. After incubation, the effect of the candidate pharmacological agent is determined in any convenient way. For cell-free binding-type assays, a separation step is often used to separate bound and unbound components. Separation may, for example, be effected by precipitation (e.g., TCA precipitation, immunoprecipitation, etc.), immobilization (e.g., on a solid substrate), followed by washing. The bound protein is conveniently detected by taking advantage of a detectable label attached to it, e.g. by measuring radioactive emission, optical or electron density, or by indirect detection using, e.g. antibody conjugates.

Suitable compounds that bind to the polypeptide or polynucleotide include polypeptide or polynucleotide fragments or small molecules, e.g., peptidomimetics. Such compounds prevent interaction and proper complex formation. Small molecule compounds, which are usually less than 10 kD molecular weight, are preferable as therapeutics since they are more likely to be permeable to cells, are less susceptible to degradation by various cellular mechanisms,. and are not as apt to elicit an immune response as would proteins or polypeptides. Small molecules include but are not limited to synthetic organic or inorganic compounds. Many pharmaceutical companies have extensive libraries of such molecules, which can be conveniently screened by using the assays of the present invention. Non-limiting examples include proteins, peptides, glycoproteins, glycopeptides, glycolipids, polysaccharides, oligosacchardies, nucleic acids, bioorganic molecules, peptidomimetics, pharmacological agents and their metabolites, transcriptional and translation control sequences, and the like.

A preferred technique for identifying compounds that bind to the polypeptide or polynucleotide utilizes a chimeric substrate (e.g., epitope-tagged fused or fused immunoadhesin) attached to a solid phase, such as the well of an assay plate. The binding of the candidate molecules, which are optionally labelled (e.g., radiolabeled), to the immobilized receptor can be measured.

Anti-Obesity Compound Identification

The present method identifies compounds useful in the treatment of obesity by selecting test compounds that exhibit binding affinity to a polynucleotide comprising a sequence of SEQ ID NO: 14 or SEQ ID NO: 16. The determination of binding affinities of such test compounds for the present polynucleotides employs in vitro assay methods known in the art. The most preferred test compound also selectively bind the polynucleotides of the present invention.

In a preferred method, test compounds that exhibit binding affinity are contacted with a first subpopulation of host cells transfected with the polynucleotide for which the test compound has affinity. The host cells are preferably primary cells, more preferably human primary cells, and most preferably, adipocytes, pre-adipocytes, mesenchymal stem cells, and progenitor cells. The host cells are transfected with the polynucleotide using methods known in the art, for example, as described above in connection with the adenoviral vectors transfection.

A second subpopulation of transfected host cells are not contacted with the test compound exhibiting binding affinity and is used as a control.

The first and second subpopulations of cells are then examined for lipid droplet formation to determine if lipid droplet formation has been inhibited in the first subpopulation relative to the second control subpopulation. Lipid droplets may be detected by a variety of methods known in the art, including microscopy, in particular, white light phase contrast microscopy, or fluorescence microscopy using Nile red stain, (Nile red: a selective fluorescent stain for neutral lipids, like intracellular lipid droplets (Greenspan, et al. (1985) J. Cell Biol. 100:965-73)). Compounds that inhibit the formation of lipid droplets are candidates for pharmaceutical development as anti-obesity drugs.

A further method for identifying a compound useful in the treatment of obesity selects test compounds that exhibit binding affinity to a polypeptide comprising a sequence of SEQ ID NO: 15.

The assay methods are similar to those described above, except that the target is the polypeptide in contrast to the polynucleotide. The host cells are transfected with an expression vector encoding the polynucleotide that encodes the polypeptide using methods known in the art. The expression vector may be any suitable expression vector that can express the polypeptide in the host cell. Preferred expression vectors include adenoviral vectors described herein to transfect such cells.

As in the foregoing assay description, a second subpopulation of transfected host cells are not contacted with the test compound exhibiting binding affinity, and is used as a control. The first and second subpopulations of cells are then examined for lipid droplet formation to determine if lipid droplet formation has been inhibited in the first subpopulation relative to the second control subpopulation.

In an alternative method for identifying such drug compounds, one or more test compounds are contacted with a corresponding number of one or more subpopulations of host cells transfected with an expression vector encoding a polynucleotide identified in the library screening methods. Examples of such polynucleotides to be used in this assay include a polynucleotide comprising a sequence of SEQ ID NO: 14 and SEQ ID NO: 16. The host cells maybe any of the host cell types used in the methods described above. The transfection may be performed using methods known in the art. Compounds that inhibit the formation of lipid droplets in the first subpopulation of cells that have been transfected (or transduction) with the expression vector relative to a second subpopulation of host cells that have not been contacted with a test compound, are selected as drug candidates for pharmaceutical development as anti-obesity pharmaceuticals.

Another method for identifying drug candidate compounds is based on the measurement, in the cellular mRNA population of the host cells, of mRNA encoded by the polynucleotide comprising a sequence of SEQ ID NO: 14 or SEQ ID NO: 16. The level of mRNA expression can be measured by a variety of methods known in the art. A drug candidate compound may be selected by comparing the mRNA expression level in the first subpopulation of host cells relative to expression of the mRNA in a second subpopulation of host cells that have not been contacted with a test compound. A decrease in the mRNA expression of the above-referenced polynucleotide would identify a compound candidate for pharmaceutical development as an anti-obesity pharmaceutical.

Identification of Compounds for the Treatment of Type II Diabetes et al

The present method identifies compounds useful in the treatment of Type II diabetes, hyperglycemia, impaired glucose tolerance, metabolic syndrome, syndrome X, dyslipidemia and insulin resistance by selecting test compounds that exhibit binding affinity to a polynucleotide comprising a sequence of SEQ ID NO: 14 or SEQ ID NO: 16 or to a polypeptide comprising a sequence of SEQ ID NO: 15.

One such method is based on polypeptide binding and contacts a test compound with a polypeptide identified in the above-described adenoviral library screening methods. Examples of such polypeptides include SEQ ID NO: 15.

The binding affinity of the test compound for the polypeptide is then determined using methods known in the art. The binding affinity may be in a nanomolar to micromolar concentrations, with nanomolar concentration preferred.

A further aspect of this method contacts a test compound that exhibits binding affinity to the target polypeptide with a first subpopulation of host cells. The host cells may be any cells that allow formation of lipid droplets. Preferred cells include pre-adipocytes, mesenchymal stem cells and progenitor cells.

Drug candidate compounds are selected from test compounds that bind to the aforesaid polypeptide and that induce an increase in expression of mRNA corresponding to a polynucleotide comprising a sequence of SEQ ID NO: 14 or of SEQ ID NO: 16 in the first subpopulation relative to expression of mRNA in a second subpopulation of host cells that has not been contacted with the test compound.

Another aspect of the present method comprises the contacting of a test compound that exhibits binding affinity for the polypeptide with a first subpopulation of host cells transfected with an expression vector encoding such polypeptide. Such first subpopulation of host cells is examined for the number and size of lipid droplets formed to determine if lipid droplet formation is enhanced in the first subpopulation relative to a second subpopulation that is not contacted with such compound. Alternatively, the first subpopulation of host cells may be transfected with a lower MOI than used in the adenoviral library assay method described above, for example, using an MOI lower that that used in the library screening method. The method can be adapted using an MOI titration to determine the activity of the test compound. Exemplary MOIs can range from 0-10%, 10-20%, 20-50% of the standard MOI. By using an MOI that is insufficient to induce lipid droplet formation in the transfected subpopulation of host cells, the present method is capable of a more sensitive determination of compounds that induce lipid drop formation.

Compounds that exhibit binding affinity for the polypeptide and enhance the formation of lipid droplets in the first subpopulation of host cells treated with said compound relative to a control untreated subpopulation of host cells are selected as drug candidate compounds. The control subpopulation of host cells is preferably transfected using the same MOI as the first subpopulation of host cells.

In another aspect of the present invention, one or more test compounds are contacted with a corresponding number of one or more first subpopulations of host cells transfected with an expression vector encoding a polynucleotide identified in the library screening methods. Examples of expression vectors to be used include expression vectors comprising a polynucleotide sequence of SEQ ID NO: 14 or SEQ ID NO: 16. The test compounds in accordance with this method may or may not have been previously identified as having any binding affinity to the aforesaid polypeptides or polynucleotides.

A drug candidate compound is selected from those compounds that enhance the formation of lipid droplets in the first subpopulation of host cells relative to a second subpopulation of host cells that have not been contacted with such compound. In an alternative aspect of the present invention, a drug candidate compound is selected from those compounds that induce an increase in expression of mRNA encoded by a polynucleotide identified using the above-described library screening method in a first subpopulation of cells relative to expression of said mRNA in a second subpopulation of host cells that has not been contacted with such test compound, The preferred mRNA populations measured in this method are encoded by a polynucleotide comprising a sequence of SEQ ID NO: 14 or SEQ ID NO: 16. The level of expression of mRNA can be measured by a variety of methods known in the art.

Depending on the size of the initial unselected library, once an adenoviral library of genes has been reduced to a reasonable number of candidates by in vitro assays, the adenoviruses can be tested in appropriate animal models. Examples of animal models that can be used include models for Alzheimer's disease, arteriosclerosis, cancer metastasis, and Parkinson's disease. In addition, transgenic animals which have altered expression of endogenous or exogenous genes including mice with gene(s) that have been inactivated, animals with cancers implanted at specific sites, human bone marrow chimeric mice such as NOD-SCID mice, and the like can be used. As additional testing is required, the stocks of candidate adenoviruses can be increased by passaging the adenoviruses under the appropriate transcomplementing conditions. Depending on the animal model used, adenoviral vectors or mixtures of pre-selected pools of adenoviral vectors can be applied or administered at appropriate sites such as lung in non-human primates (Sene, et al. (1995) Hum. Gene Ther. 6(12):1587-93) and brain of normal and apoE deficient mice (Robertson, et al. (1998) Neuroscience 82(1): 171-80.) for Alzheimer's disease (Walker, et al. (1997) Brain Res. Brain Res. Rev. 25(1):70-84) and Parkinson disease models (Hockman, et al. (1971) Brain Res. 35(2):613-8; Zigmond and Stricker, (1984) Life Sci. 35(1):5-18). The adenoviral vectors or mixtures of pre-selected pools of adenoviral vectors can also be injected in the blood stream for liver disease models including liver failure and Wilson disease (Cuthbert, (1995) J. Investig. Med. 43(4):323-36; Karrer, et al. (1984) Curr. Surg. 41(6):464-7) and tumor models including metastases models (Esandi, et al. (1997) Gene Ther. 4(4):280-7; Vincent, et al. (1996) J. Neurosurg. 85(4):648-54; Vincent, et al. (1996) Hum. Gene Ther. 7(2):197-205). In addition, selected adenoviral vectors can be injected directly into the bone marrow of human chimeric NOD-SCID mice (Dick, et al. (1997) Stem Cells 15 Suppl. 1:199-203; Mosier, et al. (1988) Nature 335(6187):256-9). Finally, selected adenovirus can be applied locally, for example, in vascular tissue of restenosis animal models (Karas, et al. (1992) J. Am. Coll. Cardiol. 20(2):467-74).

In the present invention, a variety of well known animal models of diabetes and obesity can be used to test the efficacy of the drug candidate compounds, including the polypeptides, nucleic acids, antibodies, and agonists and antagonists of the target molecules. The in vivo nature of such models makes them particularly predictive of responses in human patients. Animal models include both non-recombinant and recombinant (transgenic) animals. Non-recombinant animal models include, for example, rodent, e.g., murine models.

Examples of animal models that exhibit the diabetic, obese, or insulin resistant condition and that are useful in testing the efficacy of candidate therapeutic agents are described hereafter.

Defects in the metabolism of glucose and fatty acids have been linked to four loci in the rat genome using the spontaneously hypertensive rat (SHR). These four loci are described and defined in detail in U.S Pat. No. 6,322,976. The SHR rat is a widely used animal model of essential hypertension (Yamori, (1984) Handbook of Hypertension, Vol. 4. Experimental and Genetic Models of Hypertension, ed. de Jong, Elsevier Science Publishers, NY, 224-39) which has a global defect in insulin action on glucose metabolism (Rao, (1993) Diabetes 42:1364-71; Reaven, et al. (1989) Diabetes 38:1155-60; Paternostro, (1995) Cardiovasc. Res. 30:205-11; Chiappe de Cingolani, (1988) Metabolism 37:318-22. Spontaneous hypertension, dyslipidemia insulin resistance, hyperinsulinemia and increased abdominal fat, all displayed by the SHR model, are the defining features of Syndrome X. SHR may, therefore, be a model for this human condition.

As stated above, the SHR animal model of disease is useful in the study of defects in glucose- and fatty acid metabolism as well as insulin-action. Other animal models also may be of use. For example, The Goto-Kakizaki (GK) rat develops insulin resistance and non-insulin-dependent diabetes (Gauguier, et al. (1996) Nature Genetics 12:38-43; Galli, et al. (1996) Nature Genetics 12:31-7). Another animal model that is potentially of use in the invention is the Lyon hypertensive rat (Dubay, et al. (1993) Nature Genetics 3:354-7). This rat model also exhibits insulin resistance. Several strains of mice including the obese (ob), diabetic (db), agouti (Ay) strains also develop obesity and diabetes, due either to single-gene mutation or to effects in several genes.

These animal models, or cells derived from them, are useful for the expression of genes undergoing functional testing according to the invention as well as for drug targeting/screening according to the invention. For example, when placed on a high fat diet, the animal models described above develop atherosclerotic plaques. A particularly advantageous drug screening assay involyes placing the test and control animals on such a diet, administering a candidate modulator of fatty acid metabolism or insulin action to the test animals and then comparing plaque accumulation or reduction in the test animals with control animals who have been similarly fed but have not been given the candidate modulator. A difference of at least 10%, but preferably at least 20%, in plaque accumulation between the test and control populations is indicative of efficacy of the candidate modulator according to the invention. Wild-type animals and cells are also of use in drug screening assays and disease diagnosis and treatment according to the invention. In addition, transgenic animals are of use in gene expression studies and drug targeting/screening experiments; such animals may be derived from individuals having a wild-type or mutant genetic background relative to the gene under consideration.

Recombinant (transgenic) animal models can be engineered by introducing the coding portion of the genes identified herein into the genome of animals of interest, using standard techniques for producing transgenic animals. A transgenic animal is one containing a “transgene” or genetic material integrated into the genome introduced into the animal itself or an ancestor of the animal at a prenatal stage (e.g., embryonic stage). Animals that can serve as a target for transgenic manipulation include, without limitation, mice, rats, rabbits, guinea pigs, sheep, goats, pigs, and non-human primates, e.g. baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten, et al. (1985) Proc. Natl. Acad. Sci. USA 82:6148-52); gene targeting in embryonic stem cells (Thompson, et al. (1989) Cell 56:313-21); electroporation of embryos (Lo, (1983) Mol. Cell. Biol. 3:1803-14); sperm-mediated gene transfer (Lavitrano, et al. (1989) Cell 57:717-73). For review, see, for example, U.S. Pat. No. 4,736,866 and U.S. Pat. No. 4,870,009.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lakso, et al. (1992) Proc. Natl. Acad. Sci. USA 89(14):6232-36.

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock out” animals can be constructed which have a defective or altered gene encoding gene identified in the screen, as a result of homologous recombination between the endogenous gene encoding the gene and altered genomic DNA encoding the same polypeptide introduced into an embryonic cell of the animal. For example, cDNA encoding an identified gene can be used to clone genomic DNA encoding that polypeptide in accordance with established techniques. A portion of the genomic DNA encoding an identified gene can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, (1987) Cell 51(3):503-12) for a description of homologous recombination vectors). The vector is introduced into an embryonic stem cell line (e.g., by electroporation) and cells in which the introduced DNA has homologously recombined with the endogenous DNA are selected (see e.g., Li, et al. (1992) Cell 69(6): 915-26). The selected cells are then injected into a blastocyst of an animal (e.g., a mouse or rat) to form aggregation chimeras (see e.g., Bradley, (1987) in Teratocarcinomas and Embryonic Stem Cells: A Practical Approach, E. J. Robertson, ed. IRL, Oxford, 113-1521). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, by their ability to defend against certain pathological conditions and by their development of pathological conditions due to absence of the identified gene.

It may be advantageous to produce nucleic sequences possessing a substantially different codon usage, e.g., inclusion of non-naturally occurring codons from the codons present in a nucleic acid sequence identified using the methods of the present invention. Codons may be selected to increase the rate at which expression of the peptide occurs in a particular prokaryotic or eukaryotic host in accordance with the frequency with which particular codons are utilized by the host. Other reasons for substantially altering a nucleotide sequence without altering the encoded amino acid sequences include the production of RNA transcripts having more desirable properties, such as a greater half-life, than transcripts produced from the naturally occurring sequence.

The invention also encompasses production of DNA sequences that encode derivatives or fragments of the polypeptide encoded by the nucleic acid sequence identified using the methods of the present invention, entirely by synthetic chemistry. After production, the synthetic sequence may be inserted into any of the many available expression vectors and cell systems using reagents well known in the art. Moreover, synthetic chemistry may be used to introduce any desired mutations.

Also encompassed by the invention are polynucleotide sequences that are capable of hybridizing to the claimed polynucleotide sequences, and, in particular, to those shown in SEQ ID NO: 14 and SEQ ID NO: 16, and fragments thereof under various conditions of stringency. (See, e.g., Wahl and Berger, (1987) Methods Enzymol. 152:399-407; Kimmel, (1987) Methods Enzymol. 152:507-11.) For example, stringent salt concentration will ordinarily be less than about 750 mM NaCl and 75 mM trisodium citrate, preferably less than about 500 mM NaCl and 50 mM trisodium citrate, and most preferably less than about 250 nM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide.

Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations of these conditions are readily apparent to those skilled in the art.

Polynucleic Acids Identified by the Present Invention

The present invention further relates to the polynucleotides identified in the practice of the method invention described hereinabove, more particularly, those isolated nucleic acids found capable of inducing lipid droplet formation. For example, the polynucleotides having the sequences of SEQ ID NOS: 14, 16, 17 and 18 comprise polynucleotides of the present invention.

The present invention also utilizes antisense nucleic acids that can be used to down-regulate or block the expression of polypeptides capable of inducing lipid droplet formation in vitro, ex vivo or in vivo. The down regulation of gene expression using antisense nucleic acids can be achieved at the translational or transcriptional level. Antisense nucleic acids of the invention are preferably nucleic acid fragments capable of specifically hybridizing with all or part of a nucleic acid encoding a polypeptide capable of inducing lipid droplet formation or the corresponding messenger RNA. In addition, antisense nucleic acids may be designed or identified which decrease expression of the nucleic acid sequence capable of inducing lipid droplet formation by inhibiting splicing of its primary transcript. With knowledge of the structure and partial sequence of a nucleic acid capable of lipid droplet formation, such antisense nucleic acids can be designed and tested for efficacy.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its target site, the RNA-DNA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its target site. Modifications may include 2′ deoxy, O-pentoxy, O-propoxy, O-methoxy, fluoro, methoxyethoxy phosphoro-thioates, modified bases, as well as other modifications known to those of skill in the art.

Antisense nucleic acids can be prepared by expression of all or part of a sequence selected from the group consisting of SEQ ID NO: 14 and SEQ ID NO: 16, in the opposite orientation. Any length of antisense sequence is suitable for practice of the invention so long as it is capable of down-regulating or blocking expression of a nucleic acid capable of inducing lipid droplet formation. Preferably, the antisense sequence is at least about 20 nucleotides in length. The preparation and use of antisense nucleic acids, DNA encoding antisense RNAs and the use of oligo and genetic antisense is known in the art.

One approach to determining the optimum fragment of a nucleic acid sequence capable of inducing lipid droplet formation in an antisense nucleic acid treatment method involves preparing random cDNA fragments of a nucleic acid capable of inducing lipid droplet formation by mechanical shearing, enzymatic treatment, and cloning the fragment into any of the vector systems described herein. Individual clones or pools of clones are used to infect cells expressing the polypeptide and effective antisense cDNA fragments are identified by monitoring expression at the RNA or protein level.

A variety of viral-based systems, including retroviral, adeno-associated viral, and adenoviral vector systems may all be used to introduce and express antisense nucleic acids in cells. Antisense synthetic oligonucleotides may be introduced into the body of a patient in a variety of ways, as discussed below.

Reductions in the levels of polypeptides capable of inducing lipid droplet formation may be accomplished using ribozymes. Ribozymes are catalytic RNA molecules (RNA enzymes) that have separate catalytic and substrate binding domains. The substrate binding sequence combines by nucleotide complementarity and, possibly, nonhydrogen bond interactions with its target sequence. The catalytic portion cleaves the target RNA at a specific site. The substrate domain of a ribozyme can be engineered to direct it to a specified mRNA sequence. The ribozyme recognizes and then binds a target mRNA through complementary base-pairing. Once it is bound to the correct target site, the ribozyme acts enzymatically to cut the target mRNA. Cleavage of the mRNA by a ribozyme destroys its ability to direct synthesis of the corresponding polypeptide. Once the ribozyme has cleaved its target sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozyme may be chemically synthesized by combining an oligodeoxyribonucleotide with a ribozyme catalytic domain (20 nucleotides) flanked by sequences that hybridize to the target mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

To express the ribozyme of the present invention, the ribozyme sequence of the present invention is inserted into a plasmid DNA vector, a retrovirus vector, an adenovirus DNA viral vector or an adeno-associated virus vector. DNA may be delivered alone or complexed with various vehicles. The DNA, DNA/vehicle complexes, or the recombinant virus particles are locally administered to the site of treatment, as discussed below. Preferably, recombinant vectors capable of expressing the ribozymes are locally delivered as described below, and persist in target cells. Once expressed, the ribozymes cleave the target mRNA.

Ribozymes may be administered to a patient by a variety of methods. They may be added directly to target tissues, complexed with cationic lipids, packaged within liposomes, or delivered to target cells by other methods known in the art. Localized administration to the desired tissues may be done by catheter, infusion pump or stent, with or without incorporation of the ribozyme in biopolymers. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Detailed descriptions of ribozyme delivery and administration are provided in Sullivan et al. WO 94/02595.

The present invention also related to methods for expressing a polypeptide or polynucleotide identified as capable of inducing lipid droplet formation as a gene therapeutic. Preferably, the viral vectors used in the gene therapy methods of the present invention are replication defective. Such replication defective vectors will usually pack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentivirus vector systems may also be used in the practice of the present invention.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotides of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pᵣ, P₁, and trp promoters. Among the eukaryotic (including viral) promoters useful for practice of this invention are ubiquitous promoters (e.g HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters.

Additional vector systems include the non-viral systems that facilitate introduction of DNA encoding the polypeptides capable of inducing lipid droplet formation, the polynucleotides encoding these polypeptides, or antisense nucleic acids into a patient. For example, a DNA vector encoding a desired sequence can be introduced in vivo by lipofection. Synthetic cationic lipids designed to limit the difficulties encountered with liposome mediated transfection can be used to prepare liposomes for in vivo transfection of a gene encoding a marker (Felgner, et. al. (1987) Proc. Natl. Acad Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

Polypeptides Identified by the Present Invention

The present invention also relates to the polypeptides, or subfragments thereof, which have been identified by the practice of the present method invention as capable of inducing lipid droplet formation. Such polypeptides include for example, the polypeptides that are encoded by nucleic acids, including, for example, SEQ ID NO: 15, or which comprise antibodies capable of binding to such polypeptides encoded by such nucleic acids.

The polypeptides of the present invention may be prepared by recombinant technology methods, isolated from natural sources, or prepared synthetically, and may be of human, or other animal origin. The polypeptides of the present invention may be unglycosylated or modified subsequent to translation. Such modifications include glycosylation, phosphorylation, acetylation, myristoylation, methylation, isoprenylation, and palmitoylation. Preferred glycosylated polypeptides are produced in mammalian cells, and most preferably in human cells, a particular embodiment of which are the PER.C6 cells. Using recombinant DNA technology, the nucleic acid encoding the polypeptide is inserted into a suitable vector, which is inserted into a suitable host cell. The polypeptide produced by the resulting host cell is recovered and purified. The polypeptides are characterized by amino acid composition and sequence, and biological activity. Other ways to characterize the polypeptides include reproducible single molecular weight and/or multiple set of molecular weights, chromatographic response and elution profiles,

The present invention also provides antibodies directed against polypeptides capable of inducing lipid droplet formation. These antibodies may be monoclonal antibodies or polyclonal antibodies. The present invention includes chimeric, single chain, and humanized antibodies, as well as FAb fragments and the products of an FAb expression library, and Fv fragments and the products of an Fv expression library.

In certain embodiments, polyclonal antibodies may be used in the practice of the invention. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the identified gene product or a fusion protein thereof Antibodies may also be generated against the intact protein or polypeptide, or against a fragment, derivative, or epitope of the protein or polypeptide, by using for example a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the immunogenic polypeptides and proteins of the present invention. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively; the relevant cysteine residues are substituted with another amino acid residue or are deleted so as to prevent crosslinking.

Bispecific antibodies are monoclonal, preferably human or humanized, antibodies that have binding specificities for at least two different antigens. In the present case, one of the binding specificities is for the identified gene product, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in Trauneeker, et al. (1991) EMBO J. 10:3655-9.

A particularly preferred aspect of the present invention is an antibody that binds to a polypeptide capable of inducing lipid droplet formation and that is used to inhibit the activity of the polypeptide in a patient.

Antibodies as discussed above are also useful in assays for detecting or quantitating levels of a polypeptide capable of inducing lipid droplet formation. In one embodiment, these assays provide a clinical diagnosis and assessment of such polypeptides in various disease states and a method for monitoring treatment efficacy.

The present invention provides biologically compatible compositions comprising the polypeptides, polynucleotides, vectors, and antibodies of the invention. A biologically compatible composition is a composition, that may be solid, liquid, gel, or other form, in which the polypeptide, polynucleotides, vector, or antibody of the invention is maintained in an active form, e.g., in a form able to effect a biological activity. For example, a polypeptide of the invention would have lipid droplet inducing activity; a nucleic acid would be able to replicate, translate a message, or hybridize to a complementary nucleic acid; a vector would be able to transfect a target cell; an antibody would bind a polypeptide identified by the present invention. A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well known non-toxic physiologically acceptable carriers, adjuvants and vehicles as desired.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Pharmaceutical composition of the present invention comprise a replication defective recombinant viral vector and the polynucleotide identified by the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended. The formulations to be used for in vivo administration must be sterile. This is readily accomplished by filtration through sterile filtration membranes.

The active ingredients may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention provides methods of treatment, which comprise the administration to a human or other animal of an effective amount of a composition of the invention. A therapeutically effective dose refers to that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

As discussed hereinabove, recombinant viruses may be used to introduce both DNA encoding polypeptides capable of lipid droplet formation as well as antisense polynucleotides. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Ribozymes according to the present invention may be administered in a pharmaceutically acceptable carrier. Dosage levels may be adjusted based on the measured therapeutic efficacy.

Methods and Compositions for Lowering Levels of the Activity of Polypeptides Capable of Inducing Lipid Droplet Formation

The methods for decreasing the expression of a polypeptide capable of inducing lipid droplet formation and correct those conditions in which polypeptide activity contributes to a disease or disorder associated with an undesirable lipid droplet formation include but are not limited to administration of a composition comprising an antisense nucleic acid, administration of a composition comprising an intracellular binding protein such as an antibody, administration of a molecule that inhibits the activity of the polypeptide, for example, a small molecular weight molecule, including administration of a compound that down regulates expression at the level of transcription, translation or post-translation, and administration of a ribozyme which cleaves niRNA encoding the polypeptide.

Methods Utilizing Antisense Nucleic Acids

The present invention, in a particular embodiment, relates to a composition comprising an antisense polynucleotide that is used to down-regulate or block the expression of polypeptides capable of inducing lipid droplet formation. In one preferred embodiment, the nucleic acid encodes antisense RNA molecules. In this embodiment, the nucleic acid is operably linked to signals enabling expression of the nucleic acid sequence and is introduced into a cell utilizing, preferably, recombinant vector constructs, which will express the antisense nucleic acid once the vector is introduced into the cell. Examples of suitable vectors includes plasmids, adenoviruses, adeno-associated viruses, retroviruses, and herpes viruses. Preferably, the vector is an adenovirus. Most preferably, the vector is a replication defective adenovirus comprising a deletion in the E1 and/or E3 regions of the virus. In a most preferred embodiment, the antisense sequence comprises all or a portion of a polynucleotide complementary to SEQ ID NOS: 14 or 16.

In another embodiment, the antisense nucleic acid is synthesized and may be chemically modified to resist degradation by intracellular nucleases, as discussed above. Synthetic antisense oligonucleotides can be introduced to a cell using liposomes. Cellular uptake occurs when an antisense oligonucleotide is encapsulated within a liposome. With an effective delivery system, low, non-toxic concentrations of the antisense molecule can be used to inhibit translation of the target mRNA. Moreover, liposomes that are conjugated with cell-specific binding sites direct an antisense oligonucleotide to a particular tissue.

Methods Utilizing Neutralizing Antibodies and Other Binding Proteins

Another aspect of the present invention relates to the down-regulation or blocking of the expression of a polypeptide capable of inducing lipid droplet formation by the induced expression of a polynucleotide encoding an intracellular binding protein that is capable of selectively interacting with the polypeptide identified by the present method invention An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody. More preferably, the intracellular binding protein is a single chain antibody.

WO 94/02610 discloses preparation of antibodies and identification of the nucleic acid encoding a particular antibody. Using a polypeptide capable of inducing lipid droplet formation or a fragment thereof, a specific monoclonal antibody is prepared by techniques known to those skilled in the art. A vector comprising the nucleic acid encoding an intracellular binding protein, or a portion thereof, and capable of expression in a host cell is subsequently prepared for use in the method of this invention.

Alternatively, the activity of a polypeptide capable of inducing lipid droplet formation can be blocked by administration of a neutralizing antibody into the circulation. Such a neutralizing antibody can be administered directly as a protein, or it can be expressed from a vector that also codes for a secretory signal.

In another embodiment of the present invention, small molecule compounds inhibit the activity of a polypeptide that induces lipid droplet formation. These low molecular weight compounds interfere with the polypeptide's enzymatic properties or prevent its appropriate recognition by cellular binding sites.

The present invention also involves the use of small molecule compounds to down regulate expression of a polypeptide that is capable of lipid droplet formation at the level of transcription, translation or post-translation. Reporter gene systems may be used to identify such inhibitory compounds. These inhibitory compounds may be combined with a pharmaceutically acceptable carrier and administered using conventional methods known in the art.

Methods and Compositions for Increasing Levels of Activity of a Polypeptide Capable of Inducing Lipid Droplet Formation

The methods for increasing the expression or activity of a polypeptide capable of inducing lipid droplet formation polypeptide include, but are not limited to, administration of a composition comprising the polypeptide, administration of a composition comprising an expression vector that encodes the polypeptide, administration of a composition comprising a compound that enhances the enzymatic activity of the polypeptide and administration of a compound that increases expression of the gene encoding the polypeptide.

In one embodiment of the present invention, the level of activity is increased through the administration of a composition comprising the polypeptide. This composition may be administered in a convenient manner, such as by the oral, topical, intravenous, intraperitoneal, intramuscular, subcutaneous, intranasal, or intradermal routes. The composition may be administered directly or it may be encapsulated (e.g. in a lipid system, in amino acid microspheres, or in globular dendrimers). The polypeptide may, in some cases, be attached to another polymer.

In another embodiment of the present invention, the intracellular concentration of a polypeptide capable of inducing lipid droplet formation is increased through the use of gene therapy, which is through the administration of a composition comprising a nucleic acid that encodes and directs the expression of the polypeptide. In this embodiment, the polypeptide is cloned into an appropriate expression vector. Possible vector systems and promoters are discussed above. The expression vector is transferred into the target tissue using one of the vector delivery systems herein. This transfer is carried out either ex vivo in a procedure in which the nucleic acid is transferred to cells in the laboratory and the modified cells are then administered to the human or other animal, or in vivo in a procedure in which the nucleic acid is transferred directly to cells within the human or other animal. In preferred embodiments, an adenoviral vector system is used to deliver the expression vector. If desired, a tissue specific promoter is utilized in the expression vector as described above.

Non-viral vectors may be transferred into cells using any of the methods known in the art, including calcium phosphate co-precipitation, lipofection (synthetic anionic and cationic liposomes), receptor-mediated gene delivery, naked DNA injection, electroporation and bio-ballistic or particle acceleration.

Methods Utilizing a Compound that Enhances the Activity of a Polypeptide Capable of Inducing Lipid Droplet Formation

In another embodiment, the activity of the polypeptide is enhanced by agonist molecules that increase the enzymatic activity of the polypeptide or increase its appropriate recognition by cellular binding sites. These enhancer molecules may be introduced by the same methods discussed above for the administration of polypeptides.

In another embodiment, the level of a polypeptide capable of lipid droplet formation is increased through the use of small molecular weight compounds, which upregulate expression at the level of transcription, translation, or post-translation. These compounds may be administered by the same methods discussed above for the administration of polypeptides.

The subject invention discloses methods and compositions for the high throughput delivery and expression in a host of sample nucleic acid(s) encoding product(s) of unknown function. Methods are described for infecting a host with the adenoviral vectors that express the product(s) of the sample nucleic acid(s) in the host, identifying an altered phenotype relating to the formation of lipid droplets and/or adipogenesis induced in the host by the product(s) of the sample nucleic acids, and thereby assigning a function to the product(s) encoded by the sample nucleic acids. The sample nucleic acids can be, for example, synthetic oligonucleotides, DNAs, or cDNAs and can encode, for example, polypeptides, antisense nucleic acids, or GSEs. The methods can be fully automated and performed in a multiwell format to allow for convenient high throughput analysis of sample nucleic acid libraries.

The following examples describe the construction and screening, using a lipid droplet assay, of an arrayed adenoviral vector human placenta cDNA. The generation of the placental adenoviral cDNA library used in the present invention, including the construction of the plasmids, adenoviral vectors and the PER.C6 packaging cells are described in U.S. Pat. No. 6,340,595, issued Jan. 22, 2002, in, for example, Examples 1 through 27.

### EXAMPLES

### Example 1

Generation of Control Viruses

A PPARγ control virus (H5-2) is isolated from the Placenta PhenoSelect library. Sequence determination of the cDNA insert, present in the pAdapt plasmid, shows that it is identical to the published human PPARγ1 cDNA. This plasmid is used to prepare an adenovirus as described above. The control virus generated using this plasmid will be referred to as H5-2/PPARγ. Negative control viruses such as those encoding eGFP, LacZ, luciferase or empty virus are also prepared in accordance with the methods disclosed in U.S. Pat. No. 6,340,595.

### Example 2

Infection of Human Pre-Adipocytes Using Adenoviral Expression of hCAR

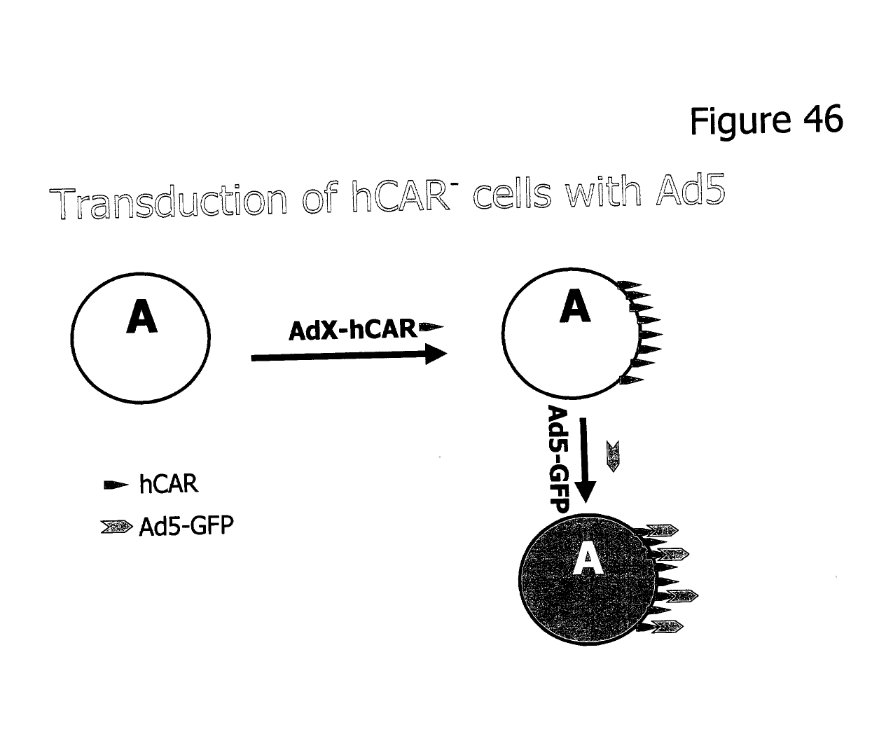

Primary human pre-adipocytes are obtained from Zen-Bio, Inc., North Carolina. These cells are difficult to transduce using Ad5C01 because they lack or have a very low expression of the receptor that mediates the infection of the Ad5C01 viruses. To circumvent this problem, adenoviruses with different fiber protein variants are used that are able to infect efficiently primary cells. These viruses, Ad5C15 or Ad5C20, code for the human Coxsackievirus and Adenovirus Receptor (hCAR) (Bergelson, et al. (1997) Science 275(5304):1320-3). Transduction with these viruses and subsequent expression of the hCAR receptor makes cells competent to transduction with Ad5C01 virus. See also FIG. 46. The use of Ad5C15-hCAR or Ad5C20-hCAR in double infections facilitates infection of primary cells using a much lower MOI for Ad5C01 than in a single infection.

The hCAR cDNA is isolated using a PCR methodology. The following hCAR-specific primers are used:

HuCARfor

(SEQ ID NO:12)

5′-GCGAAGCTTCCATGGCGCTCCTGCTGTGCTTCG-3′
HuCARrev

(SEQ ID NO:13)

5′-GCGGGATCCATCTATACTATAGACCCATCCTTGGTC-3′

The 5′ primer contains a HindIII site, and the 3′ primer a BamHI site. The hCAR cDNA is PCR amplified from a HeLa cell cDNA library (Quick clone, Clontech). A single fragment of 1119 bp is obtained and digested with the HindIII and BamHI restriction enzymes. pIPspAdapt6 vector (described in U.S. Pat. No. 6,340,595) is digested with the same enzymes, gel-purified and used to ligate to the digested PCR hCAR fragment.

All viruses described in this example, and the following examples, have the Ad5 genome backbone with the E1A, E1B and E2A genes deleted. Only the viruses Ad5C15-hCAR and Ad5C20-hCAR, described in this paragraph, have a fiber modification (C15 or C20) and do not have the E2A gene deleted in their genome. The most used virus is the Ad5C01 variant.

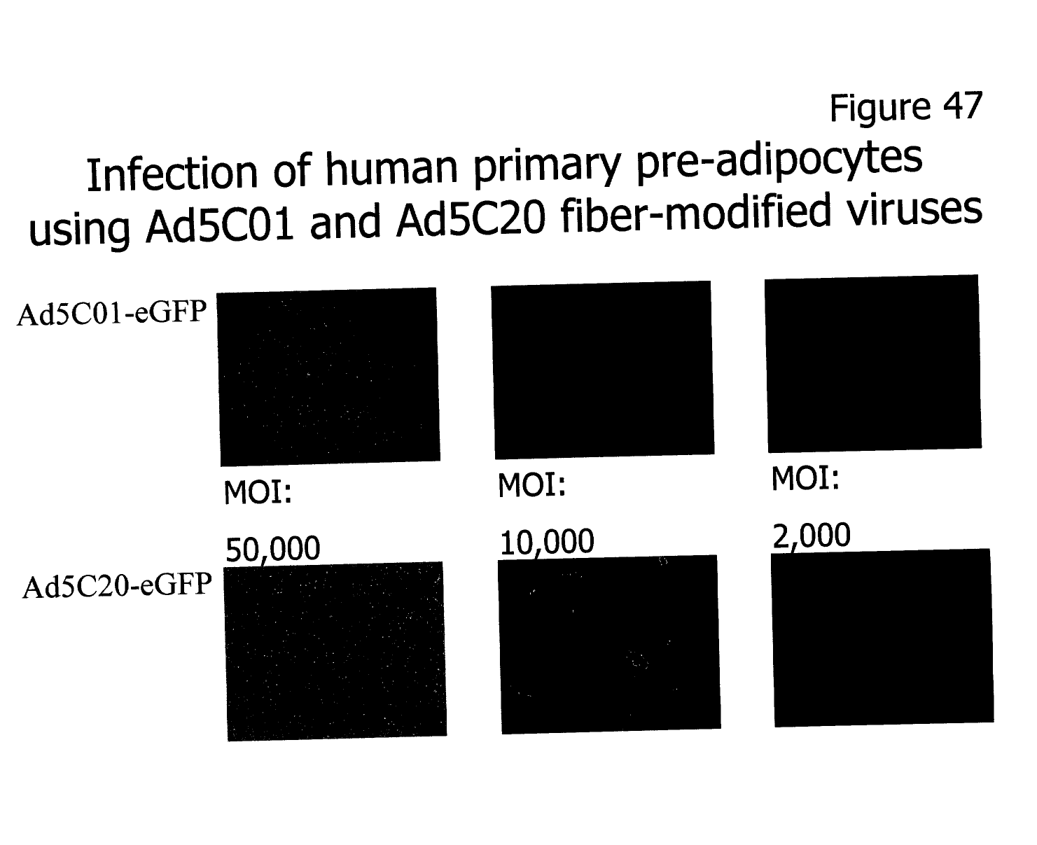

From FIG. 47, it is clear that the classical Ad5C01 virus infects these cells with a low efficiency while Ad5C20 infects them at a high efficiency. We therefore use Ad5C20 at an MOI of 25,000 to transduce these cells with the hCAR cDNA, in a coinfection with an Ad5C01 virus at an MOI of 2,000 encoding for example eGFP, PPARγ or a virus from the PhenoSelect library. This co-infection results in a higher transduction efficiency for the Ad5C01 virus compared to a single infection using only an Ad5C01 virus.

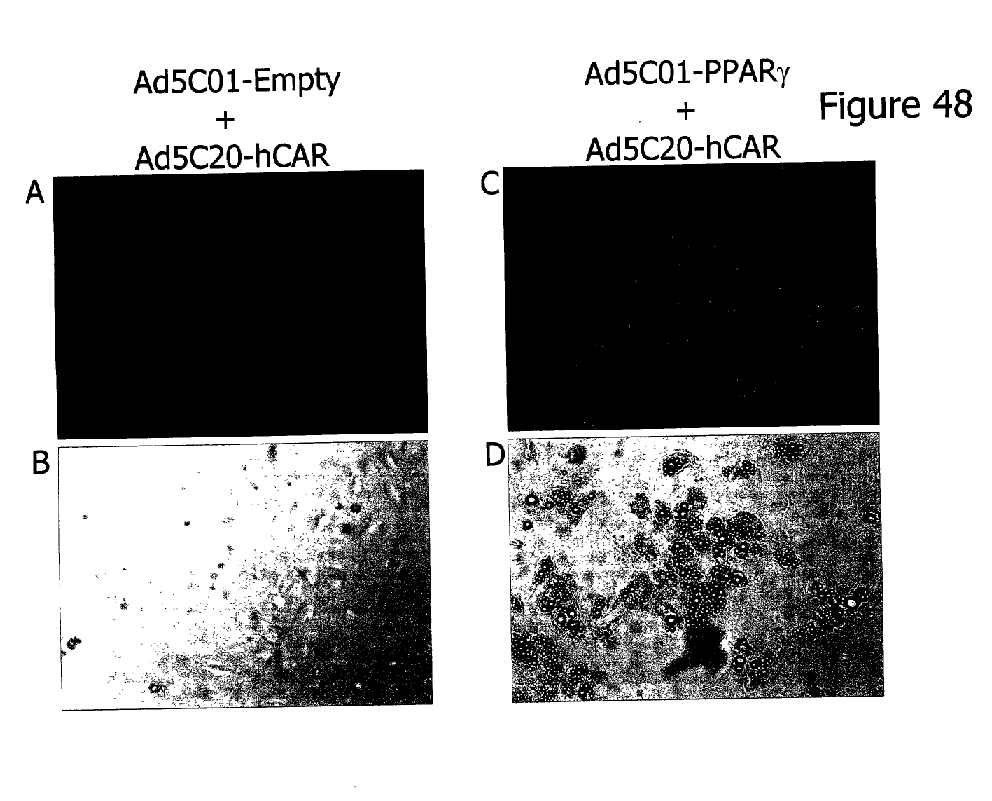

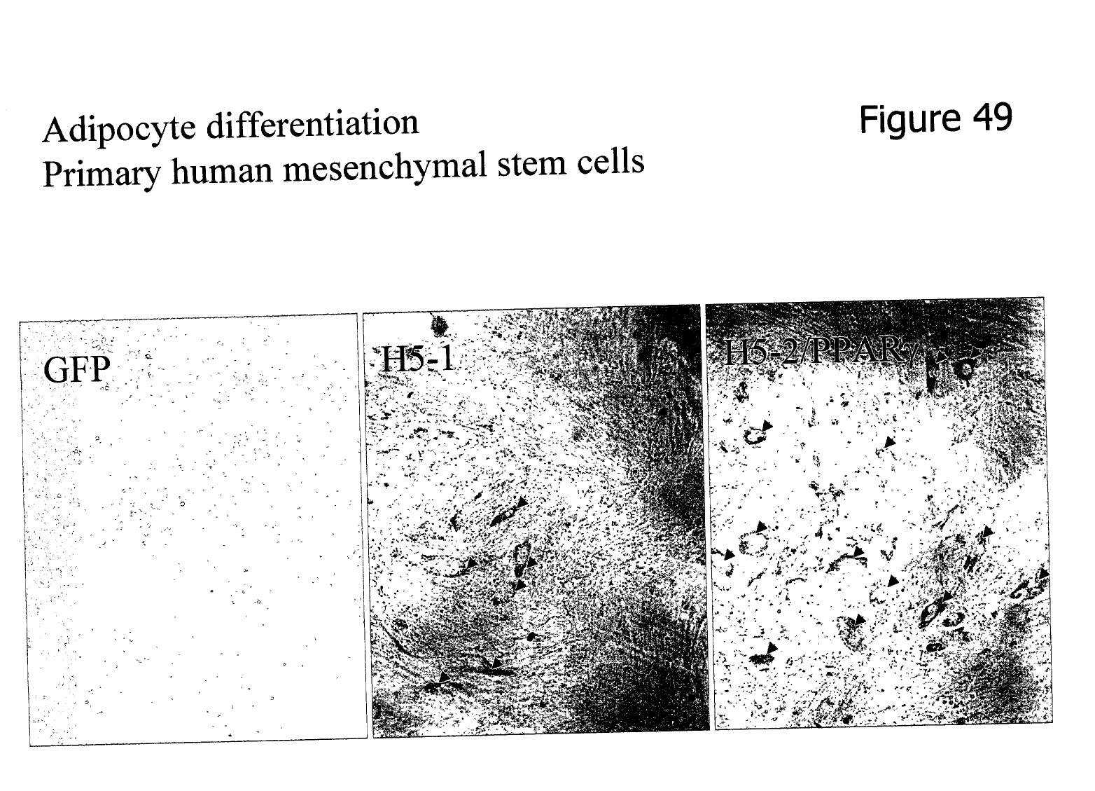

In FIG. 48, the proof-of-principle experiment for this assay is presented. Primary human pre-adipocytes are infected using Ad5C20-hCAR virus together with Ad5C01- PPARγ virus. Seven days later, formation of lipid droplets is clearly seen using white light microscopy. When the lipophilic fluorophore Nile Red is added to the medium, the fluorophore accumulates in cell membranes and lipid droplets. The latter are then easily visualized using fluorescence microscopy.

### Example 3

Screening Method for the Detection of Lipid Droplet Formation

Mesenchymal precursor cells (or pre-adipocytes) are determined to differentiate into fat cells (adipocytes) in the presence of transcription factors that act as specific gene expression switches (e.g. PPARγ) or in the presence of ligands and compounds (e.g. indomethacin or TZDs) that activate transcription factors such as PPARγ.

Primary human pre-adipocytes are seeded at 1,000 cells/well in black 384 well plates with clear bottom (Costar or Nunc). Positive and negative Ad5C01 control viruses are used to coinfect the cells using Ad5C20-hCAR at MOIs described in Example 2. Formation of PPARγ-induced lipid droplet formation is followed over time. At seven days post infection, lipid droplet formation is consistently observed.

### Example 4

Lipid Droplet Screen with 25,000 Placenta PhenoSelect Adenoviruses Protocol for Screening the PhenoSelect Library

On day 0, 1000 primary pre-adipocytes are seeded in 60 μl medium, in each well of a black 384 well plate with clear bottom (Costar or Nunc). One day later, Ad5C20-hCAR is added to each well at an MOI of 25,000 as follows: Ad5C20-hCAR viral stocks are diluted to 25,000,000 viral particles per 5 μl. 290 μl of this dilution is dispensed into each well of a 96 well plate. The virus is transferred from each well of the 96 well plate to 4 wells of the 384 well plates in a volume of 5 μl per well of the 384 well plate. Pipetting is performed using a Hydra290 96 channel dispensor.

On the same day, control viruses or viruses from the PhenoSelect library are added to the hCAR transfected wells according to the following procedure: Plates harbouring control viruses or PhenoSelect library viruses are allowed to thaw at room temperature. Two μl of control virus or library virus are transferred to the 384 well plate containing the MPC cells using a Hydra100 96 channel dispenser. The viruses from the control plates are screened in duplicate, while the viruses from the PhenoSelect library are screened in singular fashion. The plates containing the freshly infected cells are then incubated at 37° C. Seven days after infecting the cells, plates are analyzed using a white light phase-contrast microscope to score for wells containing lipid droplet-harbouring cells.

25,000 placenta PhenoSelect viruses are screened. Three viruses clearly induce lipid droplet formation. Several other adenoviruses (approximately 38) also induced this phenotype but to a lesser extent and were discarded.

### Example 5

Rescreen Hits from Lipid Droplet Screen

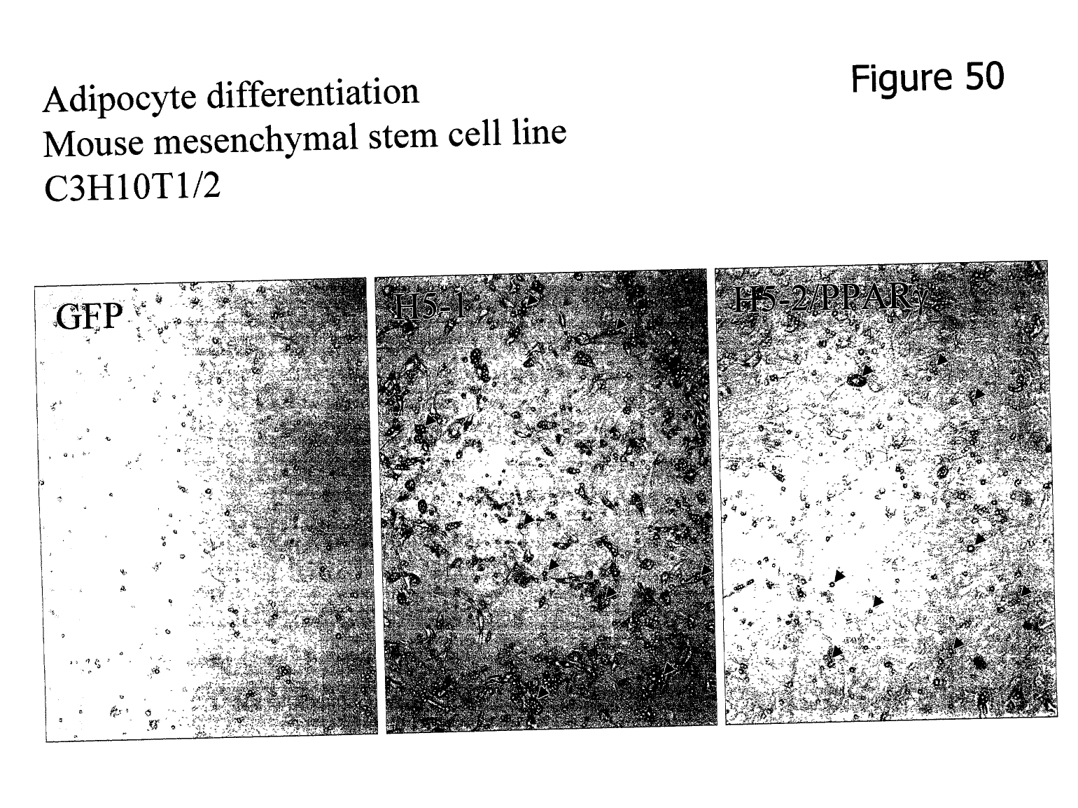

The viruses that scored positive in the initial screen are identified and samples of those identified viruses reselected from the PhenoSelect library. These selected viruses are transfected into 4×10⁴ PerC6/E2A cells seeded in 96 well plates (200 μl of DMEM+10% FBS) using 1 μl from the original PhenoSelect library plates. Six days later, when most infected wells exhibit CPE, the plates are transferred from the 37° C. incubator to a −80° C. freezer. Aliquots of these viruses are made upon thawing in V-bottom 96 well plates and then rescreened under the same conditions as used for the primary screen (see Example 4). This rescreening is performed using primary human pre-adipocytes, and a murine C3H10T½ mesenchymal progenitor cell line (see e.g. FIG. 50).

### Example 6

Sequence Identification of Validated Hits

For sequencing and tracking purposes, the cDNAs expressed by the hit adenoviruses are amplified by PCR using primers complementary to sequences flanking the multiple cloning site of the pAdapt plasmid (see U.S. Pat. No. 6,340,595). The following protocol is applied to obtain these PCR fragments: PerC6/E2A cells are seeded in 96 well plates at a density of 40,000 cells per well in 180 μl PerC6/E2A medium. Cells are incubated overnight at 39° C. in a 10% CO₂ humidified incubator. One day later, cells are infected with the ‘hit viruses’ using 2 μl of crude cell lysate material from the repropagation step. Cells are then incubated at 34° C., 10% CO₂ until appearance of starting of CPE (as revealed by the swelling and rounding up of the cells, typically 2 to 3 days post infection). The supernatant is then removed from the cells and 50 μl of lysis buffer (1× Expand High Fidelity buffer with MgCl₂ supplemented with 1 mg/ml proteinase K and 0.45% Tween-20 is added to the cells). After mixing cell lysates are then transferred to sterile micro-centrifuge tubes and incubated at 55° C. for 2 h followed by a 15 min inactivation step at 95° C. 5 μl of the cell lysates is then added to a PCR master mix composed of 5 μl 10× Expand High Fidelity buffer with MgCl₂, 1 μl of dNTP mix (10 mM for each dNTP), 1 μl of pClip-FOR primer (10 μM stock, sequence: 5′ GGT GGG AGG TCT ATA TAA GC; SEQ ID NO: 19), 1 μl of pAdapt-REV primer (10 μM stock, sequence: 5′ GGA CAA ACC ACA ACT AGA ATG C; SEQ ID NO: 20), 0.75 μl of Expand High Fidelity DNA polymerase (3.5 U/μl) and 36.25 μl of H₂O. PCR is performed using a PE Biosystems GeneAmp PCR system 9700 as follows: the PCR mixture (50 μl in total) is incubated at 95° C. for 5 min; at 95° C. for 30 sec; 55° C. for 30 sec; 68° C. for 4 min, and thi is repeated for 35 cycles. A final incubation at 68° C. is applied for 7 min. The amplification products are resolved on a 0.8% agarose gel containing 0.5 μg/ml ethidium bromide and their length estimated by comparison with the migration of a standard DNA ladder. For this purpose, 10 μl of PCR mixture is mixed with 2 μl of 6× gel loading buffer.

### Example 7

RT-PCR Analysis of Genes Induced by the Lipid Droplet Assay Hits RNA Extraction

Total RNA from adenovirally transduced primary human pre-adipocytes is extracted 7 days post infection using TRIzol® reagent according to the manufacturer's recommendations.

Cells (24-well plate) are homogenized in 300 μl TRIzol® reagent. Phases are separated by the addition of chloroform and RNA is isopropanol precipitated from the aqueous phase. The RNA pellet is washed with 70% ethanol, air-dried and dissolved in 50 μl DEPC treated H₂O.

The concentration is determined using RiboGreen RNA Quantitation Reagent (Molecular Probes). All RNA extracts are diluted to 40 ng/μl.

Reverse transcription PCR

Gene specific primers are designed to specifically amplify aP2, SREBP1c, C/EBPα,β,γ,δ. Human β-act,in primers (Clontech) are used to check for RNA integrity and quantity.

RT-PCR is the most sensitive technique to determine the presence or absence of RNA templates. The Titan One Tube RT-PCR kit (Roche) is a one step reaction system using AMV for first strand synthesis and Expand High Fidelity enzyme mixture for PCR. The protocol is followed as essentially described by the manufacturer.

TABLE 1

| Mix 1 | |
|---|---|
| H2O (DEPC-treated) | 7.125 |
| dNTPmix 2.5 mM each | 2.0 |
| DTT | 1.25 |
| RNase inh 40 U/μl | 0.125 |
| FOR 10 μM | 0.5 |
| REV 10 μM | 0.5 |
| template | 1.0 |
| Mix 2 | |
| H2O (DEPC-treated) | 7.0 |
| 5xRT-PCR buffer | 5.0 |
| Enzyme mix | 0.5 |

| RT-PCR | cycle program | |
|---|---|---|
| 50° C. | 30 min | |
| 94° C. | 2 min | |
| 94° C. | 30 s | |
| 60° C. | 30 s | 10x |
| 68° C. | 60 s | |
| 94° C. | 30 s | |
| 60° C. | 30 s | 25x |
| 68° C. | 90 s | |
| 68° C. | 7 min | |
| 10° C. | hold | |

Negative and positive controls (eGFP, PPARγ) and the assay hits are subjected to this analysis.

### Example 8

The Validated Hits

Hit H5-1

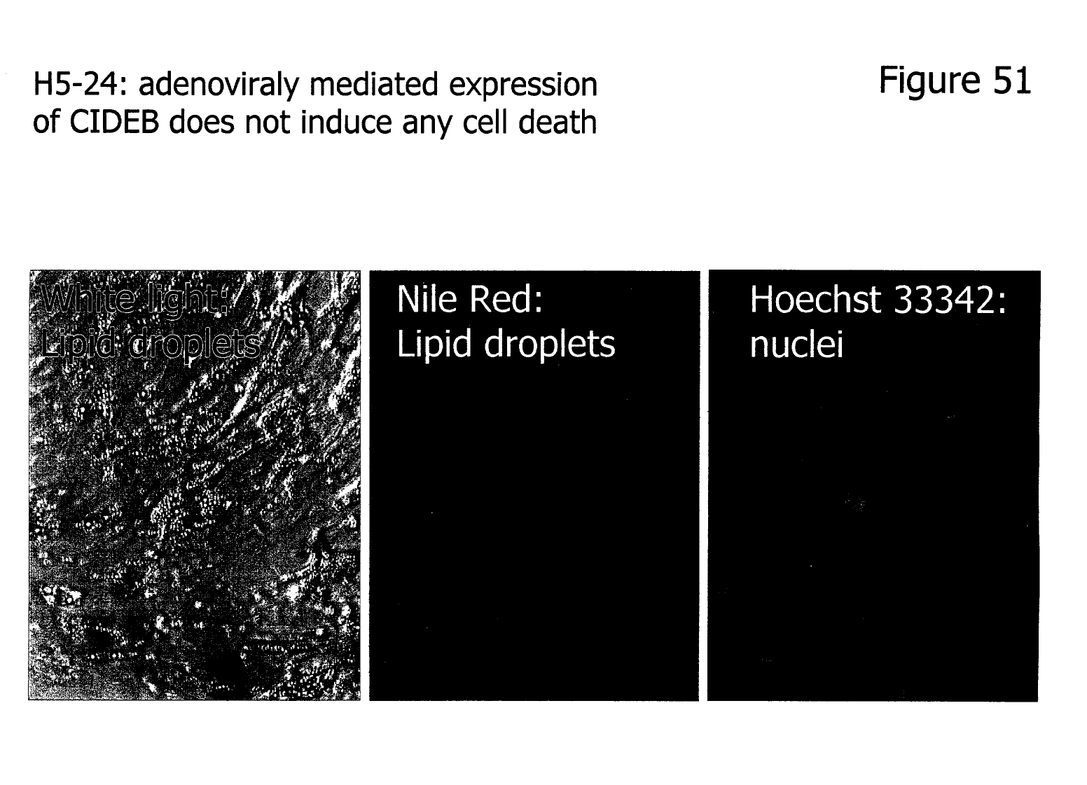

The cDNA sequence identified as H5-1 induces lipid droplet formation in the above-described assay. This cDNA, 1215 nt long, matches with the last 74 nucleotides of the open reading frame of the human presenilin 1 gene and with the 3′-UTR of this gene. (FIG. 51, SEQ ID NO: 14)

Hit H5-24

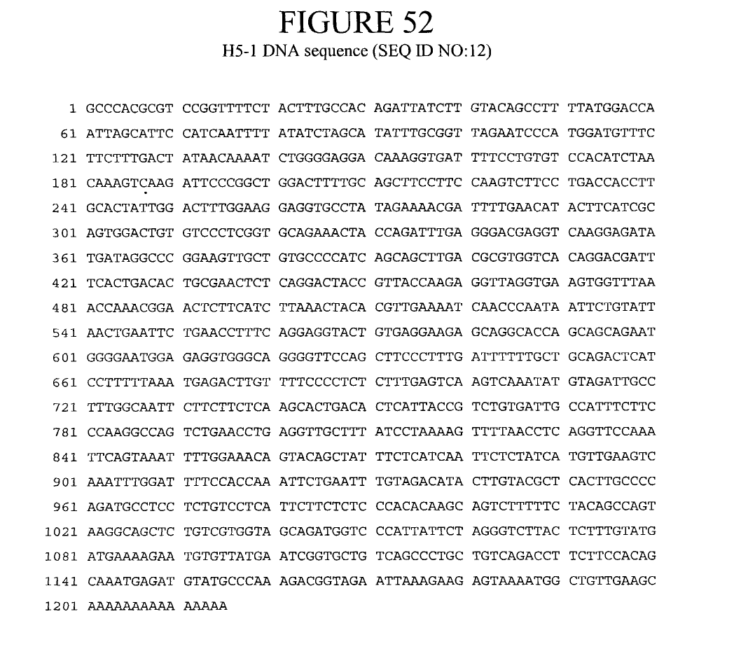

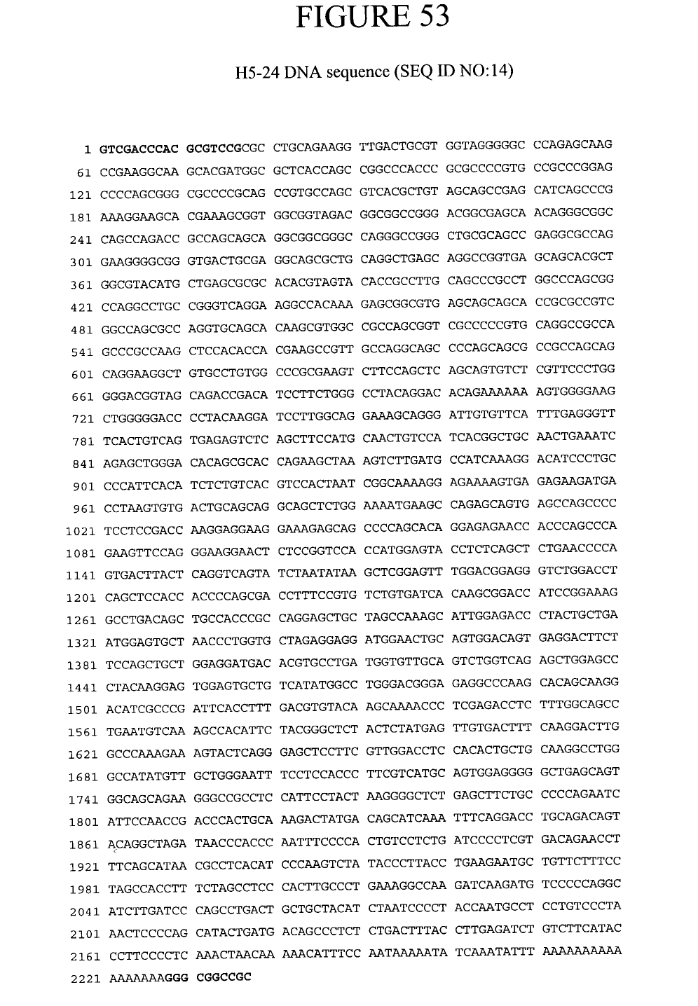

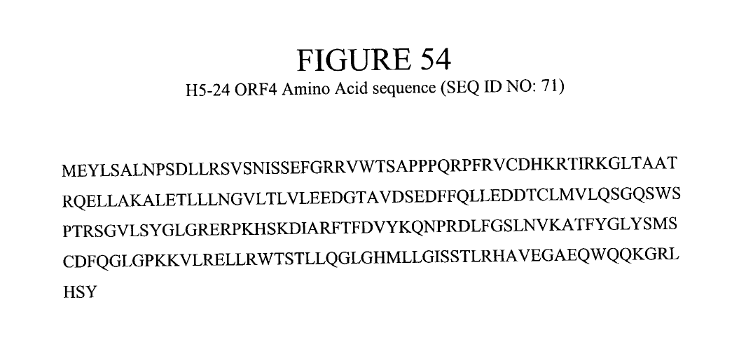

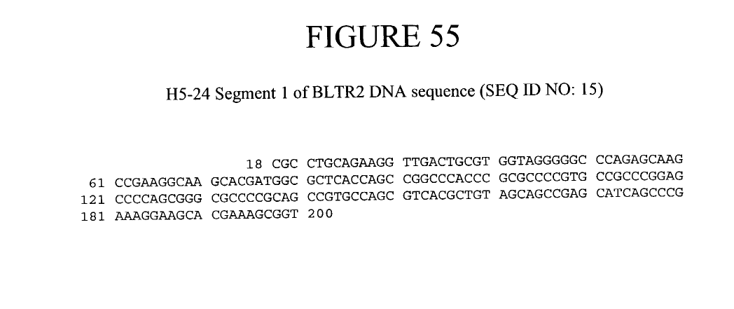

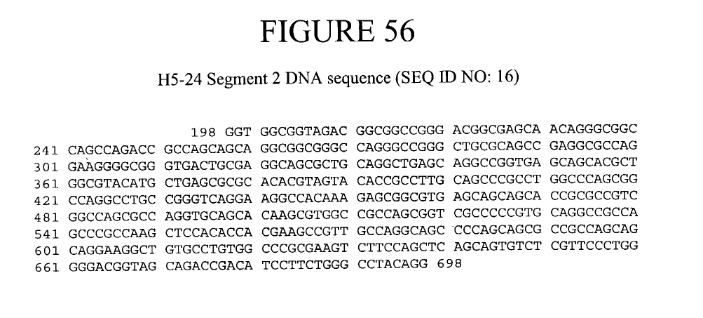

The cDNA sequence identified as H5-24 induces lipid droplet formation in the above-described assay. The cDNA sequence of H5-24 (FIG. 52, SEQ ID NO: 16) corresponds to Homo sapiens cell death-inducing DFFA-like effector b (CIDE-B), mRNA (XM_{—}_033245, NM_{—}014430) (Annotation in Genbank: CAD; Region: Domains present in proteins implicated in post-mortem DNA fragmentation). The proteins, CIDE-A and CIDE-B, which are expressed in both liver and spleen, upon overexpression in 293T cells, induce apoptosis (Inohara, et al. (1998) EMBO J. 17(9):2526-33). The gene encoding this cDNA is located on human chromosome 14q11.2-q12. This chromosomal region also contains the genes for BLTR2 and LTBR4, 2 seven transmembrane receptors that bind leukotriene B4.

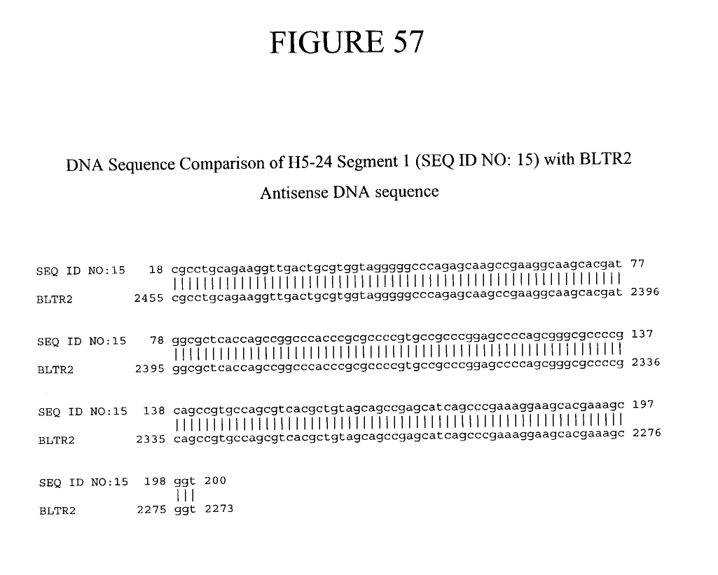

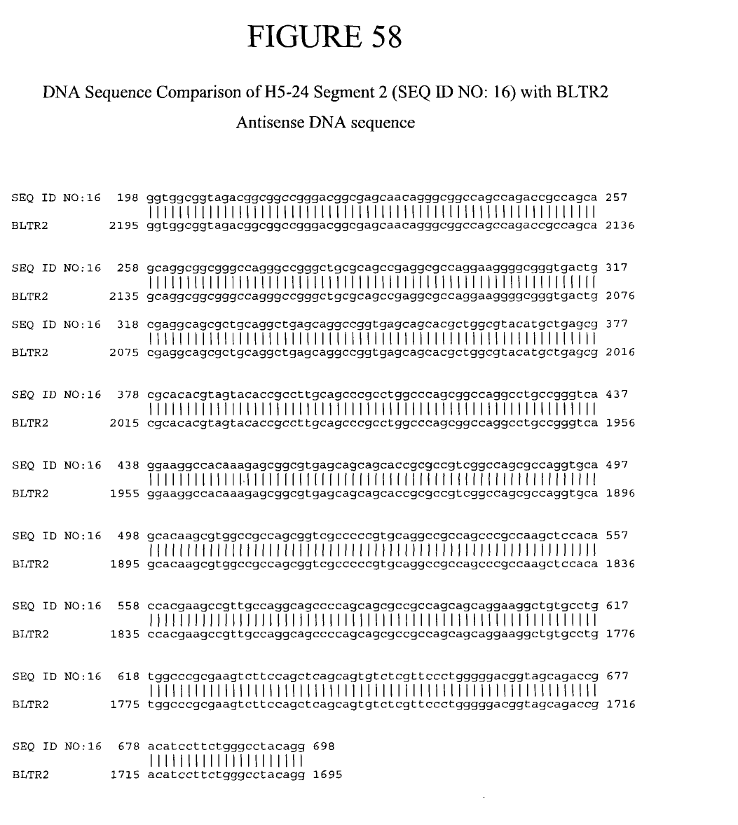

The overexpression of H5-24 does not induce any cell death (see FIG. 51), but induces lipid drop formation. Since there are two upstream regions of H5-24 polynucleotide antisense to BLTR2 (see FIGS. 57 and 58), H5-24 exogenous expression can downregulate BLTR2 mRNA and protein expression.

### Example 9

Analysis of Hits for Activity as Secreted Proteins

Cells, hereafter “termed producer cells”, are infected using viruses identified as “hits” in the lipid droplet formation assay as well as control viruses that induce or do not induce lipid droplet formation. The conditioned medium is harvested 3 or 4 days post infection (dpi) and added to freshly seeded primary human pre-adipocytes. If the conditioned medium contains secreted proteins that induce lipid droplet formation, this will be identified 7 days after adding the conditioned medium to human pre-adipocytes by analyzing lipid droplet formation.

HeLa or U2OS producer cells are cultured in DMEM 10% FBS. 5000 HeLa cells/ well or 5000 U2OS cells/well (384 well plate) are plated in 60 μl medium. Four hours later, the cells are infected with 1 μl of adenoviral stock solutions. Two or 3 days later, 384 well plates containing 1000 pre-adipocytes/well are seeded in 30 μl of medium. One day after seeding the pre-adipocytes, 40 μl of the conditioned medium, harvested from the HeLa or U2OS producer cells is transferred to the corresponding well of the 384 well plates containing the pre-adipocytes, using the 96-channel Hydra dispenser. Seven days after transferring the supernatants lipid droplet formation is analysed using white light phase contrast microscopy.

### Example 10

Human FAb Phage Display Selection of Antibodies Against Validated Hits

Phage displaying human FAb fragments encompassing the light and heavy variable and constant regions are employed to isolate antibodies that bind to the protein identified herein (characterized by SEQ ID NO: 15). A human FAb phage display library is constructed in a phage display vector such as pCES1 a vector derived from pCANTAB6 (McCafferty, et al. (1994) Appl. Biochem. Biotech. 47:157-73). The library is constructed in the filamentous E. coli phage m13 and the FAb sequences are displayed as N-terminal fusion proteins with the minor coat protein pIII. The library can have a complexity of more or less than 10¹⁰ different sequences.

Three types of targets can be used to select for polypeptide-displaying phages that bind to the amino acid epitopes present in the sequences of SEQ ID NO: 14 or SEQ ID NO: 16.

First, a predicted extracellular or otherwise accessible domain encoded by sequences of SEQ ID NO: 14 or SEQ ID NO: 16 is synthesized as a synthetic peptide. The N-terminus of this peptide is biotinylated and followed by three amino acid linker residues KRR, followed by the predicted sequence of encoded by sequences of SEQ ID NO: 14 or SEQ ID NO: 16, respectively.

Second, a fusion protein is made of a portion of or the complete polypeptide encoded by sequences of SEQ ID NO: 14 or SEQ ID NO: 16 in frame with the ORF of glutathione-S-transferase (GST) or maltose-binding protein or His6 or another tag and expressed in E. coli. Alternatively, a His6 or another tag is fused in frame with the ORF of SEQ ID NO: 14 or SEQ ID NO: 16 and expressed in a mammalian expression system such as PER. C6/E2A. Fusion proteins are then purified using, for example, NiNTA columns for His6-tagged proteins (Qiagen) or glutathione resin (Pharmacia) for GST-tagged proteins.

To select for FAb displaying phages that bind to polypeptides encoded by sequences of SEQ ID NO: 14 or SEQ ID NO: 16, the following selection procedure is employed. A pool of FAb displaying phage is selected out of a complex mixture of a high number of different FAb displaying phages in four rounds by their ability to bind with significant affinity to a biotinylated peptide or to a purified fusion protein that has been expressed in E. coli or in a mammalian expression system such as PER.C6/E2A. The collection of selected FAb displaying phage is further decreased by the next selection procedure: the FAb displaying phage are further selected in three rounds for their ability to bind to polypeptides encoded by sequences of SEQ ID NO: 14 or SEQ ID NO: 16 present in cell lysates from cells overexpressing SEQ ID NO: 14 or SEQ ID NO: 16. For selection on biotinylated peptide 250 μl of FAb library (or eluted phage from the previous round) is mixed with 250 μl 4% Marvel in PBS and equilibrated while rotating at RT for 1 hour. Subsequently biotinylated hCAT1 peptide (20-500 nM in H₂O) is added. This mix is incubated on the rotator at RT for 1 hour before 250 ml equilibrated streptavidin-dynabeads in 2% Marvel in PBS is added. After incubation on a rotator at RT for 15 min the beads with the bound phage are washed 5 times with PBS/2% Marvel/0.1% Tween, 5 times with PBS /0.1% Tween and 5 times with PBS. Then the phage are eluted by incubation with 0.1M Triethylamine on a rotator at RT for 10 min and neutralised in 1 M Tris-HCl pH 7.4.

The eluted phages are titered and amplified in TG1 before the next selection.

The pools of the last various selection rounds are tested for binding to the biotinylated peptides or preferably the fusion or purified full length proteins in a specific ELISA and also for cell binding by flow cytometric analysis where appropriate. Once FAb displaying clones are isolated, double strand phagemid DNA is prepared and used to determine the nucleotide and deduced amino acid sequence of the displayed variable heavy and light chains.

The FAb phages or antibodies derived thereof are used as diagnostic tools, for example in immunohistochemistry, as research tools, for example in affinity chromatography, as therapeutic antibodies directly, or for the generation of therapeutic antibodies by generating anti-idiotypic antibodies.

### Example 11

Screening for Compounds that Affect Lipid Droplet Formation

Polynucleotide SEQ ID NO: 14 or SEQ ID NO: 16 or polypeptide of SEQ ID NO: 15 is attached to the bottom of the wells of a 96-well plate by incubating the polypeptide or polynucleotide in the wells overnight at 4° C. Alternatively, the wells are first coated with composition of polylysine that facilitates binding of the polypeptide or polynucleotides.

Following attachment of the biopolymer, samples from a library of test compounds are added to the wells and incubated for a sufficient time and temperature to facilitate binding using an appropriate binding buffer known in the art. Following this incubation, the wells are washed with an appropriate washing solution at 4° C. The stringency of the washing steps is varied by increasing or decreasing salt and/or detergent concentrations in the wash. Detection of binding is accomplished by using antibodies (RIA, ELISA), biotintylation, biotin-streptavidin binding, and radioisotopes. The concentration of the sample library compounds is also varied to calculate a binding affinity by Scatchard analysis.

Binding to the polypeptide or polynucleotides identifies a “lead compound”. Once a lead compound is identified the screening process is repeated using compounds chemically related to the lead compound to identify compounds with the tightest binding affinities. Selected compounds having binding affinity are further tested in one of the two following assays.

Lipid Droplet Assay: Compounds that bind to the polynucleotide or polypeptide are tested for their effects on lipid droplet formation. In general, a cell that expresses a polynucleotide of SEQ ID NO: 14 or SEQ ID NO: 16 is treated with a binding compound. The treatment with the compound can occur pre-transfection with the polynucleotide sequence (see day 0 and 1 below), post-transfection (see days 1 to 8 below), or concurrently with transfection (see day 1 below). After transfection and incubation with the compound, lipid droplet formation is assessed.

On day 0, 1000 primary pre-adipocytes are seeded in 60 μl medium, in each well of a black 384 well plate with clear bottom (Costar or Nunc). One day later, Ad5C20-hCAR is added to each well at an MOI of 25,000 as follows: Ad5C20-hCAR viral stocks are diluted to 25,000,000 viral particles per 5 μl. 290 μl of this dilution is dispensed into each well of a 96 well plate. The virus is transferred from each well of the 96 well plate to 4 wells of the 384 well plates in a volume of 5 μl per well of the 384 well plate. Pipetting is performed using a Hydra290 96 channel dispenser. On the same day, control viruses or viruses comprising SEQ ID NO: 14 or SEQ ID NO: 16 are added to the hCAR transfected wells according to the following procedure: Plates harbouring control viruses or SEQ ID NO: 14 or SEQ ID NO: 16 are allowed to thaw at room temperature. Two μl of control virus or SEQ ID NO: 14 or SEQ ID NO: 16 virus are transferred to the 384 well plate containing the MPC cells using a Hydra100 96 channel dispenser. The viruses from the control plates are screened in duplicate, while the viruses from the PhenoSelect library are screened in singular fashion. The plates containing the freshly infected cells are then incubated at 37° C. Seven days after infecting the cells, plates are analysed using a white light phase-contrast microscope to score for wells containing lipid droplet-harbouring cells. The binding compounds identified in the previous step can be added on Day 0, Day 1, or on any of the days after transfection with the virus containing SEQ ID NO: 14 or SEQ ID NO: 16.

In the case where lipid droplet formation is enhanced as compared to cells that have not been exposed to the binding compound, the compound is classified as an agonist. In the case where lipid droplet formation is inhibited as compared to cells that have not been exposed to the binding compound, the compound is classified as an antagonist.

mRNA Expression Assay: On day 0, 1000 primary pre-adipocytes are seeded in 60 μl medium in the wells of a black 384-well plate with clear bottom (Costar or Nunc). The cells are plated in duplicate so that RNA is isolated from a first set of plates while lipid droplet formation is assessed in the second set of plates. One day later, the binding compound is added to the medium of both sets of plates at a concentration ranging from 1 nM to 1mM. Seven days after addition of the compound, the second set of plates is analyzed for lipid droplet formation using a white light phase-contrast microscope to score for wells containing lipid droplet-harboring cells. One day after addition of the compound, the cells of the first set are lysed and the RNA from the cells is extracted. Extraction is performed as described in Maniatis, et al. (1989) Molecular Cloning: A Laboratory Manual, 2nd ed., or alternatively a commercially available kit (e.g., Qiagen) is used. RNA isolated from the cells is used as template for PCR using primers specific to SEQ ID NO: 14 or SEQ ID NO: 16 to determine if the compound induces this DNA sequence.

As an alternative, the above experiment can be done at a larger scale in 96- or 24-well plates so that mRNA encoded by SEQ ID NO: 14 or SEQ ID NO: 16 is isolated, and detected by RNase protection assay or northern blotting. Alternatively, cell lysates are isolated and subjected to SDS-PAGE electrophoresis, transferred to membranes, and immuoblotted to detect expression of polypeptides encoded by SEQ ID NO: 14 or SEQ ID NO: 16.

### Example 12

In Vivo Analysis of Hits from the Lipid Droplet Screen

Down regulation and over expression of SEQ ID NO: 14 or SEQ ID NO: 16 are tested in transgenic animal models.

For down regulating expression of SEQ ID NO: 14 or SEQ ID NO: 16, knockout animals, preferably mice, are generated according to established procedures. One or more exons of the genes encoding SEQ ID NO: 14 or SEQ ID NO: 16 are deleted by homologous recombination in mouse ES cells. These ES cells have been isolated from a limited number of homozygous strains of inbred lab mice well-suited to derive knock-out mice and are well known for those skilled in the art. Removal of one or more exons is checked by techniques such as southern blotting and the diploid state of ES cells is checked by cytogenetic techniques. Knockout ES cells harbouring the expected microdeletion and the expected number of chromosomes are then used to derive mice, according to established procedures. Resulting chimeric mice are then used to start a colony of knockout mice where the mice can be hetero- or homozygous for the allele in which one or more exons of the gene corresponding to SEQ ID NO: 14 or SEQ ID NO: 16 are deleted. Both hetero- and homozygous knock-out mice are then used to study e.g. adipogenesis, circulating levels of insulin, glucose, fatty acids, glucose uptake by adipose tissue and muscle in these mice, in comparison with wild-type mice, i.e. mice from the same inbred homozygous strain that have the gene corresponding to SEQ ID NO: 14 or SEQ ID NO: 16 intact. The absence of expression of SEQ ID NO: 14 or SEQ ID NO: 16 is studied by western blotting and northern blotting, performed on tissues, including bone tissue of wild-type and knock-out animals. The absence of expression of SEQ ID NO: 14 or SEQ ID NO: 16 on adipose tissue biology is measured in a number of ways: physical parameters such as the presence of all white and brown adipose tissue, normally seen in healthy wild-type animals are analysed. Additionally, the muscle, liver and the circulation are examined in more detail for glucose, fatty acids and for proteins like leptin, TNFα, resistin, adiponectin, etc.

For over expressing SEQ ID NO: 14 and SEQ ID NO: 16 in vivo, preferably in mice, the following procedure is followed: subclone SEQ ID NO: 14 or SEQ ID NO: 16 into a eukaryotic expression plasmid, downstream of a ubiquitously expressed promoter or, preferably, downstream of a promoter allowing for expression only in the bone compartment. The plasmid containing the above-mentioned promoter and SEQ ID NO: 14 or SEQ ID NO: 16 is then used to derive transgenic mice according to established procedures. Homozygous mouse strains, well suited to derive transgenic mice, such as the FVB strain are used. Exogenous expression of SEQ ID NO: 14 or SEQ ID NO: 16 is analysed using southern blot, allowing an estimation of the copy number of the expression cassette, integrated in the mouse genome and also by northern or western blotting, if antibodies are available. The effect of the exogenous expression of SEQ ID NO: 14 or SEQ ID NO: 16 on adipose tissue biology and on obesity and diabetes is analysed as described above for knockout animals.

All publications and patent applications are herein incorporated by reference to the same extent as if each individual publication or patent application is specifically and individually indicated to be incorporated by reference.

The invention now having been fully described, it will be apparent to one of ordinary skill in the art that many changes and modifications may be made thereto without departing from the spirit or scope of the appended claims.

We claim:

1. A method for identifying a unique nucleic acid capable of inducing lipid droplet formation in a cell, wherein said unique nucleic acid is present in a library, said method comprising:
(a) providing a library of a multitude of unique expressible nucleic acids, said library including a multiplicity of compartments, each of said compartments consisting essentially of one or more adenoviral vector comprising at least one unique nucleic acid of said library in an aqueous medium, wherein said adenoviral vector is capable of introducing said nucleic acid into a host cell, is capable of expressing the product of said nucleic acid in said host cell, and is deleted in a portion of the adenoviral genome necessary for replication thereof in said host cell;
(b) transducing a multiplicity of host cells with at least one adenoviral vector comprising at least one unique nucleic acid from said library;
(c) incubating said host cells to allow expression of the product of said nucleic acid; and
(d) determining if a lipid droplet is formed in said cell.

2. The method of claim 1 wherein step (d) comprises observing said host cell to identify lipid droplet formation in said host cell relative to a host cell that has not been transduced with an adenoviral vector comprising said nucleic acid.

3. The method of claim 1, wherein the function of the expression product of all of said unique expressible nucleic acids in said library is unknown at the time said library is first made.

4. The method of claim 1, wherein none of said compartments contain any adenoviral vector capable of replication except in a packaging cell containing said deleted portion of said adenoviral genome.

5. The method of claim 1, wherein said host cell is a eukaryotic cell.

6. The method of claim 1, wherein at least one compartment comprises at least two adenoviral vectors.

7. The method of claim 1, wherein each of said compartments consists essentially of one said adenoviral vector.

8. The method of claim 4, wherein each of said compartments contains from about 0.01×10¹⁰ to about 10×10¹⁰ pfu of said adenoviral vector per ml of aqueous medium.

9. The method of claim 8, wherein each of said compartments further contains the cellular debris from packaging cell lysate.

10. The method of claim 4, wherein said adenoviral vector is a minimal vector.

11. The method of claim 10, wherein said minimal vector comprises an adenovirus encapsidation signal or a functional part, derivative and/or analogue thereof, and at least one copy of at least a functional part or a derivative of an adenoviral ITR.

12. The method of claim 4, wherein said adenoviral vector comprises adenoviral genomic sequence deleted for sequence encoding the E1-region proteins.

13. The method of claim 11 wherein said minimal vector further comprises an adeno-associated virus terminal repeat or a functional part, derivative and/or analogue thereof

14. The method of claim 12, wherein said adenoviral vector is further deleted for sequence encoding the E2A-region proteins, or the E2B region proteins or the complete E2 region proteins.

15. The method of claim 1, wherein said adenoviral vector further comprises adenovirus genomic sequence encoding adenoviral fiber proteins from at least two serotypes of adenovirus.

16. The method according to claim 1, wherein said multiplicity of cells is divided over a multiplicity of compartments, each said compartment comprising at least one vector.

17. The method according to claim 1, further comprising selecting at least one vector comprising a unique nucleic acid capable of inducing lipid droplet formation in a cell.

18. The method according to claim 1, wherein at least one of said performed steps is automated.

19. A method for obtaining an expressible nucleic acid capable of inducing lipid droplet formation when expressed in a cell, said method comprising:
(a) performing the method of claim 1;
determining which compartment in said library contains an adenoviral vector comprising a unique nucleic acid capable of inducing lipid droplet formation; and
obtaining said vector from said compartment.

20. The method of claim 1 wherein said host cell is a pre-adipocyte.

21. The method of claim 1 wherein said host cell is selected from the group consisting of pre-adipocytes, mesenchymal stem cells and progenitor cells.

22. The method of claim 1 wherein said lipid droplets are detected microscopically.

23. The method of claim 22 wherein said microscopy is white light phase contrast microscopy.

24. The assay of claim 22 wherein said microscopy is fluorescence microscopy.

25. The method of claim 1 further comprising transducing the host cells in step (b) with an adenovirus encoding the receptor for adenovirus subtype 5(hCAR) wherein said receptor is expressed in said host cell.

26. A method for determining whether the expression product of a nucleic acid, capable of inducing lipid droplet formation in a cell transfected with said nucleic acid, is secreted by said cell, comprising:
(a) infecting producer cells in a medium with an adenoviral vector comprising a unique nucleic acid capable of inducing lipid droplet formation;
(b) combining said medium with test cells that have not been infected with said vector; and
(c) determining if lipid droplets are formed in said test cells.

27. The method of claim 26 wherein said test cells are primary human pre-adipocytes.

28. The method of claim 9, wherein the contents of each said compartment is capable of transfecting said host cell and expressing the product of each said unique nucleic acid in said host cell.

29. The method of claim 28, wherein each said compartment is capable of providing from about 10 to about 20 aliquots of said adenoviral vector.

30. The method of claim 11, wherein said minimal vector comprises a regulatable promoter operably linked to said unique nucleic acid.

31. The method of claim 12, wherein said adenoviral vector comprises a regulatable promoter operably linked to said unique nucleic acid.

32. The method of claim 12, wherein said adenoviral vector is further deleted for the adenoviral E3 -region or a functional part thereof

33. The method of claim 14, wherein said adenoviral vector is further deleted for the adenoviral E3 -region or a functional part thereof

34. The method of claim 32, wherein said adenoviral vector is further deleted for the adenoviral E4-region or a functional part thereof

35. The method of claim 33, wherein said adenoviral vector is further deleted for the adenoviral E4-region or a functional part thereof

36. The method of claim 30, wherein said promote r is repressed by an adenoviral E1 gene product.

37. The method of claim 31, wherein said promoter is repressed by an adenoviral E1 gene product.

38. The method of claim 36, wherein said promoter is an AP1 dependent promoter.

39. The method of claim 37, wherein said promoter is an AP1 dependent promoter.

40. A method according to claim 1, wherein said adenoviral vector is packaged into an adenoviral capsid.

41. The method of claim 1 wherein said unique expressible nucleic acid is derived from the group consisting of mammals, fish, nematodes, insects, yeasts, fungi, bacteria and plants.

42. The method of claim 41 wherein said library of unique nucleic acid is derived from human placenta mRNA.

43. The method of claim 41 wherein said library of unique nucleic acid is derived from zebrafish mRNAs.

44. A method for identifying a unique nucleic acid capable of inducing lipid droplet formation in a cell, wherein said unique nucleic acid is present in a library, said method comprising:
(a) growing a plurality of cell cultures containing at least one cell, said one cell expressing adenoviral sequence consisting essentially of E1-region sequences and expressing one or more functional gene products encoded by at least one adenoviral region selected from an E2A region and an E4 region; and
(b) transfecting, under conditions whereby said recombinant adenovirus vector library is produced, said at least one cell in each of said plurality of cell cultures with
i) an adapter plasmid comprising adenoviral sequence coding, in operable configuration, for a functional Inverted Terminal Repeat, a functional encapsidation signal, and sequences sufficient to allow for homologous recombination with a first recombinant nucleic acid, and not coding for E1 region sequences which overlap with E1 region sequences in said at least one cell, for E1 region sequences which overlap with E1 region sequences in a first recombinant nucleic acid, for E2B region sequences other than essential E2B sequences, for E2A region sequences, for E3 region sequences and for E4 region sequences, and further comprises a unique nucleic acid sequence and promoter operatively linked to said unique nucleic acid sequence; and
ii) a first recombinant nucleic acid comprising adenoviral sequence coding, in operable configuration, for a functional adenoviral Inverted Terminal Repeat and for sequences sufficient for replication in said at least one cell, but not comprising adenoviral E1 region sequences which overlap with E1 sequences in said at least one cell, and not comprising E2A region sequences or E4 region sequences expressed in said plurality of cells which would otherwise lead to production of replication competent adenovirus wherein said first recombinant nucleic acid has sufficient overlap with said adapter plasmid to provide for homologous recombination resulting in production of recombinant adenoviral vectors in said at least one cell;
(c) incubating said plurality of cells under conditions which result in the lysis of said plurality of cells facilitating the release of said recombinant adenoviral vectors containing said unique nucleic acid; and
(d) transferring an aliquot of said adenoviral vectors into a corresponding plurality of host cell cultures consisting of cells in which said vectors do not replicate, but in which said nucleic acids are expressible;
(e) incubating said host cells to allow expression of the product of said nucleic acid; and
(f) observing said host cell for the presence of a lipid droplet

45. A method according to claim 44, wherein said lipid droplets are detected microscopically.

46. A method for identifying a drug candidate compound useful in the treatment of obesity, said method comprising:
(a) contacting one or more test compound with a polynucleotide comprising a sequence of SEQ ID NOS: 14, 16, 17 or 18, or the corresponding antisense sequence thereof,
(b) determining the binding affinity of said one or more test compound to said polynucleotide,
(c) contacting a first subpopulation of host cells transfected with said polynucleotide with one or more of said test compound that exhibits binding affinity for said polynucleotide, and
(d) identifying, from said one or more test compounds, a candidate compound that inhibits the formation of lipid droplets in said first subpopulation of host cells relative to a second subpopulation of said transfected host cells that have not been contacted with said candidate compound.

47. A method according to claim 46 wherein said test compound comprises a polynucleotide or a polypeptide.

48. A method for identifying a drug candidate compound useful in the treatment of obesity, said method comprising:
(a) contacting one or more test compound with a polypeptide expression product encoded by the polynucleotide comprising a sequence of SEQ ID NOS: 14 or 16,
(b) determining the binding affinity of said test compound to said polypeptide,
(c) contacting a first subpopulation of host cells transfected with a polynucleotide expression vector coding for said polypeptide with one or more of said test compound that exhibits binding affinity for said polypeptide, and
(d) identifying, from said one or more test compounds, a candidate compound that inhibits the formation of lipid droplets in said first subpopulation of host cells relative to a second subpopulation of said transfected host cells that have not been contacted with said candidate compound.

49. A method according to claim 48 wherein said test compound comprises a polypeptide comprising the sequence of SEQ ID NO: 15.

50. A method for identifying a drug candidate compound useful in the treatment of obesity comprising:
(a) contacting one or more test compounds with a corresponding number of one or more first subpopulations of host cell transfected with an expression vector encoding a polynucleotide comprising a sequence of SEQ ID NOS: 14, 16, 17 or 18.

51. A method according to claim 50 further comprising
(b) selecting, from said one or more test compounds, a candidate compound that inhibits the formation of lipid droplets in said first subpopulation of host cell relative to a second subpopulation of said transfected host cell that have not been contacted with any test compound.

52. A method according to claim 50 comprising
(b) selecting a candidate compound that results in an decrease in the expression of mRNA encoded by a polynucleotide comprising a sequence of SEQ ID NOS: 14, 16, 17 or 18 in said first subpopulation of host cell relative to the expression of said mRNA in a second subpopulation of transfected host cells that has not been contacted with any test compound.

53. A method for identifying a drug candidate compound useful in the treatment of a disease state wherein said disease state is selected from the group consisting of type II diabetes, hyperglycemia, impaired glucose tolerance, metabolic syndrome, syndrome X, dyslipidemia and insulin resistance, said method comprising:
(a) contacting a test compound with an expression product of the polynucleotide comprising a sequence of SEQ ID NOS: 14 or 16 or the corresponding antisense sequence thereof, and
(b) determining the binding affinity of said test compound to said expression product.

54. A method according to claim 53 comprising:
(c) contacting said test compound that exhibits binding affinity to said expression product with a first subpopulation of host cell, and
(d) selecting as a candidate compound, said test compound that causes an increase in the expression of mRNA encoded by a polynucleotide comprising a sequence of SEQ ID NOS: 14, 16, 17 or 18 in said first subpopulation of host cells relative to the expression of mRNA in a second subpopulation of host cell that has not been contacted with said compound.

55. A method according to claim 53 further comprising:
(c) contacting said test compound that exhibits binding affinity for said expression product with a first subpopulation of host cells transfected with an expression vector encoding said polypeptide, and
(d) selecting as a candidate compound, said compound that exhibits said binding affinity and that enhances the formation of lipid droplets in said first subpopulation of host cells relative to a second subpopulation of said transfected host cells that have not been contacted with said binding affinity compound.

56. A method for identifying a drug candidate compound useful in the treatment of a disease state wherein said disease state is selected from the group consisting of type II diabetes, hyperglycemia, impaired glucose tolerance, metabolic syndrome, syndrome X, dyslipidemia and insulin resistance, said method comprising:
(a) contacting one or more test compounds with a corresponding number of one or more first subpopulations of host cell transfected with an expression vector encoding a polynucleotide comprising a sequence of SEQ ID NOS: 14, 16, 17 or 18.

57. A method according to 56 further comprising
(b) selecting, from said one or more test compounds, a candidate compound that enhances the formation of lipid droplets in said first subpopulation of host cell relative to a second subpopulation of said host cell that have not been contacted with any test compound.

58. A method according to claim 56 comprising
(b) selecting a candidate compound that results in an increase in the expression of mRNA encoded by a polynucleotide comprising a sequence of SEQ ID NOS: 14, 16, 17 or 18 in said first subpopulation of cell relative to the expression of said mRNA in a second subpopulation of host cells that has not been contacted with said any test compound.

59. An isolated antibody that specifically binds to the polypeptide expression product encoded by the polynucleotide comprising the sequence of SEQ ID NO: 14.

60. A pharmaceutical composition comprising a polypeptide expression product encoded by the polynucleotide comprising the sequence of SEQ ID NO: 14 and a pharmaceutically acceptable carrier.

61. A pharmaceutical composition comprising a polynucleotide sequence of SEQ ID NOS: 14, 16, 17 or 18 and a pharmaceutically acceptable carrier.

62. A pharmaceutical composition comprising a polynucleotide complementary to the sequence of SEQ ID NOS: 14, 16, 17 or 18 and a pharmaceutically acceptable carrier.

63. A method for treating a disease state selected from the group consisting of type II diabetes, hyperglycemia, impaired glucose tolerance, metabolic syndrome, syndrome X, dyslipidemia and insulin resistance, said method comprising administering to a patient in need of such treatment an effective adipogenesis amount of the composition of claim 60.

64. A method for treating a disease state selected from the group consisting of type II diabetes, hyperglycemia, impaired glucose tolerance, metabolic syndrome, syndrome X, dyslipidemia and insulin resistance, said method comprising administering to a patient in need of such treatment an effective adipogenesis amount of the composition of claim 61.

65. A method for treating obesity, said method comprising administering to a patient in need of such treatment an effective anti-obesity amount of the composition of claim 62.

66. An expression vector comprising a polynucleotide sequence of SEQ ID NOS: 14, 16, 17 or 18.

67. A method for treating a disease state selected from the group consisting of type II diabetes, hyperglycemia, impaired glucose tolerance, metabolic syndrome, syndrome X, dyslipidemia and insulin resistance, said method comprising administering to host cells of a patient in need of such treatment an effective adipogenesis amount of the expression vector of claim 66.

68. An expression vector comprising a polynucleotide complementary to a polynucleotide sequence of SEQ ID NOS: 14, 16, 17, or 18 wherein said vector is capable of expressing said polynucleotide.

69. A method of treating obesity comprising administering to host cells of a patient in need of such treatment the expression vector of claim 68.

70. A method according to claim 50 wherein said cells are primary cells.

71. A method according to claim 70, wherein said primary cells are selected from the group consisting of adipocytes, pre-adipocytes, mesenchymal stem cells, and progenitor cells.


SEQUENCE LISTING (20 sequences)

SEQ ID NO: 1 - DNA, 21 bp, Artificial Sequence
  Description of Artificial Sequenceprimer
cgtgtagtgtatttatacccg

SEQ ID NO: 2 - DNA, 21 bp, Artificial Sequence
  Description of Artificial Sequenceprimer
tcgtcactgggtggaaagcca

SEQ ID NO: 3 - DNA, 21 bp, Artificial Sequence
  Description of Artificial Sequenceprimer
tacccgccgtcctaaaatggc

SEQ ID NO: 4 - DNA, 21 bp, Artificial Sequence
  Description of Artificial Sequenceprimer
gcctccatggaggtcagatgt

SEQ ID NO: 5 - DNA, 20 bp, Artificial Sequence
  Description of Artificial Sequenceprimer
gcttgagcccgagacatgtc

SEQ ID NO: 6 - DNA, 24 bp, Artificial Sequence
  Description of Artificial Sequenceprimer
cccctcgagctcaatctgtatctt

SEQ ID NO: 7 - DNA, 27 bp, Artificial Sequence
  Description of Artificial Sequenceprimer
gggggatccgaacttgtttattgcagc

SEQ ID NO: 8 - DNA, 25 bp, Artificial Sequence
  Description of Artificial Sequenceprimer
gggagatctagacatgataagatac

SEQ ID NO: 9 - DNA, 27 bp, Artificial Sequence
  Description of Artificial Sequenceprimer
gggagatctgtactgaaatgtgtgggc

SEQ ID NO: 10 - DNA, 24 bp, Artificial Sequence
  Description of Artificial Sequenceprimer
ggaggctgcagtctccaacggcgt

SEQ ID NO: 11 - DNA, 45 bp, Artificial Sequence
  Description of Artificial Sequenceprimer
gtacactgacctagtgccgcccgggcaaagcccgggcggcactag

SEQ ID NO: 12 - DNA, 33 bp, Artificial Sequence
  Description of Artificial Sequenceartificial primer with HindIII site
gcgaagcttccatggcgctcctgctgtgcttcg

SEQ ID NO: 13 - DNA, 36 bp, Artificial Sequence
  Description of Artificial Sequenceartificial primer with BamHi site
gcgggatccatctatactatagacccatccttgctc

SEQ ID NO: 14 - DNA, 1215 bp, Human
gcccacgcgtccggttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacctttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcaaaaaaaaaaaaaaa

SEQ ID NO: 15 - protein, 219 aa, Human
MEYLSALNPSDLLRSVSNISSEFGRRVWTSAPPPQRPFRVCDHKRTIRKGLTAATRQELLAKALETLLLNGVLTLVLEEDGTAVDSEDFFQLLEDDTCLMVLQSGQSWSPTRSGVLSYGLGRERPKHSKDIARFTFDVYKQNPRDLFGSLNVKATFYGLYSMSCDFQGLGPKKVLRELLRWTSTLLQGLGHMLLGISSTLRHAVEGAEQYQQKGRLHSY

SEQ ID NO: 16 - DNA, 2237 bp, Human
gtcgacccacgcgtccgcgcctgcagaaggttgactgcgtggtagggggcccagagcaagccgaaggcaagcacgatggcgctcaccagccggcccacccgcgccccgtgccgcccggagccccagcgggcgccccgcagccgtgccagcgtcacgctgtagcagccgagcatcagcccgaaaggaagcacgaaagcggtggcggtagacggcggccgggacggcgagcaacagggcggccagccagaccgccagcagcaggcggcgggccagggccgggctgcgcagccgaggcgccaggaaggggcgggtgactgcgaggcagcgctgcaggctgagcaggccggtgagcagcacgctggcgtacatgctgagcgcgcacacgtagtacaccgccttgcagcccgcctggcccagcggccaggcctgccgggtcaggaaggccacaaagagcggcgtgagcagcagcaccgcgccgtcggccagcgccaggtgcagcacaagcgtggccgccagcggtcgcccccgtgcaggccgccagcccgccaagctccacaccacgaagccgttgccaggcagccccagcagcgccgccagcagcaggaaggctgtgcctgtggcccgcgaagtcttccagctcagcagtgtctcgttccctgggggacggtagcagaccgacatccttctgggcctacaggacacagaaaaaaagtggggaagctgggggacccctacaaggatccttggcaggaaagcagggattgtgttcatttgagggtttcactgtcagtgagagtctcagcttccatgcaactgtccatcacggctgcaactgaaatcagagctgggacacagcgcaccagaagctaaagtcttgatgccatcaaaggacatccctgccccattcacatctctgtcacgtccactaatcggcaaaaggagaaaagtgagagaagatgacctaagtgtgactgcagcaggcagctctggaaaatgaagccagagcagtgagccagcccctcctccgaccaaggaggaaggaaagagcagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatggcctgggacgggagaggcccaagcacagcaaggacatcgcccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctagcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaaaaaaaaaaaaaaaaagggcggccgc

SEQ ID NO: 17 - DNA, 183 bp, Human
cgcctgcagaaggttgactgcgtggtagggggcccagagcaagccgaaggcaagcacgatggcgctcaccagccggcccacccgcgccccgtgccgcccggagccccagcgggcgccccgcagccgtgccagcgtcacgctgtagcagccgagcatcagcccgaaaggaagcacgaaagcggt

SEQ ID NO: 18 - DNA, 500 bp, Human
ggtggcggtagacggcggccgggacggcgagcaacagggcggccagccagaccgccagcagcaggcggcgggccagggccgggctgcgcagccgaggcgccaggaaggggcgggtgactgcgaggcagcgctgcaggctgagcaggccggtgagcagcacgctggcgtacatgctgagcgcgcacacgtagtacaccgccttgcagcccgcctggcccagcggccaggcctgccgggtcaggaaggccacaaagagcggcgtgagcagcagcaccgcgccgtcggccagcgccaggtgcagcacaagcgtggccgccagcggtcgcccccgtgcaggccgccagcccgccaagctccacaccacgaagccgttgccaggcagccccagcagcgccgccagcagcaggaaggctgtgcctgtggccccgaagtcttccagctcagcagtgtctcgttccctgggggacggtagcagaccgacatccttctgggcctacagg

SEQ ID NO: 19 - DNA, 20 bp, Artificial Sequence
  Description of Artificial Sequenceartificial sequencing primer
ggtgggaggtctatataagc

SEQ ID NO: 20 - DNA, 22 bp, Artificial Sequence
  Description of Artificial Sequenceartificial sequencing primer
ggacaaaccacaactagaatgc